(19) United States
(12) Patent Application Publication
(10) Pub. No.: US 2005/0136431 A1
(43) Pub. Date: Jun. 23, 2005

(54) Methods of screening compositions for G protein-coupled receptor desensitization inhibitory activity

(76) Inventor: Robert H. Oakley, Durham, NC (US)

Correspondence Address: HUTCHISON & MASON PLLC, PO box 31686, RALEIGH, NC, 27612, US

(21) Appl. No.: 10/901,772

(22) Filed: Jul. 29, 2004

Related U.S. Application Data
(63) Continuation of application No. 10/633,438, filed on Aug. 1, 2003.
Application No. 10/633,438 is a continuation-in-part of application No. 09/993,844, filed on Nov. 5, 2001.
(60) Provisional application No. 60/245,772, filed on Nov. 3, 2000.
(60) Provisional application No. 60/260,363, filed on Jan. 8, 2001.

(51) Int. Cl. C12Q 1/68; G01N 33/53; G01N 33/567

(52) U.S. Cl. 435/6; 435/7.2

(57) ABSTRACT

The methods of the present invention allow the screening of a test composition for non-receptor-specific GPCR desensitization inhibitory activity. The methods involve screening a test composition for an indication of GPCR desensitization inhibitory activity against two or more GPCRs that are different from each other. When there is an indication that a particular test composition has GPCR desensitization inhibitory activity with respect to each of the two or more GPCRs that are different from one another, then, according to the present invention, there is an indication that the test composition has non-receptor-specific GPCR desensitization inhibitory activity.

### RELATED APPLICATIONS

This application is a continuation of U.S. application Ser. No. 10/633,438, filed Aug. 1, 2003, which is a continuation-in-part of U.S. application Ser. No. 09/993,844, filed Nov. 5, 2001, which claims the benefit of U.S. Provisional Application No. 60/245,772, filed Nov. 3, 2000 and U.S. Provisional Application No. 60/260,363, filed Jan. 8, 2001; the entire contents of these applications are hereby incorporated by reference herein.

### FIELD OF THE INVENTION

The present invention generally relates to methods for screening test compositions for G protein-coupled receptor desensitization inhibitory activity, and more particularly relates to screening test compositions for G protein coupled receptor desensitization inhibitory activity that is not specific to a particular receptor.

### BACKGROUND OF THE INVENTION

G protein-coupled receptors (GPCRs) are cell surface proteins that translate hormone or ligand binding into intracellular signals. GPCRs are found in all animals, insects, and plants. GPCR signaling plays a pivotal role in regulating various physiological functions including phototransduction, olfaction, neurotransmission, vascular tone, cardiac output, digestion, pain, and fluid and electrolyte balance. Although they are involved in various physiological functions, GPCRs share a number of common structural features. They contain seven membrane domains bridged by alternating intracellular and extracellular loops and an intracellular carboxyl-terminal tail of variable length.

The magnitude of the physiological responses controlled by GPCRs is linked to the balance between GPCR signaling and signal termination. The signaling of GPCRs is controlled by a family of intracellular proteins called arresting. Arrestins bind activated GPCRs, including those that have been agonist-activated and especially those that have been phosphorylated by G protein-coupled receptor kinases (GRKs).

GPCRs have been implicated in a number of disease states, including, but not limited to: cardiac indications such as angina pectoris, essential hypertension, myocardial infarction, supraventricular and ventricular arrhythmias, congestive heart failure, atherosclerosis, renal failure, diabetes, respiratory indications such as asthma, chronic bronchitis, bronchospasm, emphysema, airway obstruction, upper respiratory indications such as rhinitis, seasonal allergies, inflammatory disease, inflammation in response to injury, rheumatoid arthritis, chronic inflammatory bowel disease, glaucoma, hypergastrinemia, gastrointestinal indications such as acid/peptic disorder, erosive esophagitis, gastrointestinal hypersecretion, mastocytosis, gastrointestinal reflux, peptic ulcer, Zollinger-Ellison syndrome, pain, obesity, bulimia nervosa, depression, obsessive-compulsive disorder, organ malformations (for example, cardiac malformations), neurodegenerative diseases such as Parkinson's Disease and Alzheimer's Disease, multiple sclerosis, Epstein-Barr infection and cancer.

Receptors, including GPCRs, have historically been targets for drug discovery and therapeutic agents because they bind ligands, hormones, and drugs with high specificity. Approximately fifty percent of the therapeutic drugs in use today target or interact directly with GPCRs. See, e.g., Jurgen Drews, (2000) “Drug Discovery: A Historical Perspective,” Science 287:1960-1964.

A common limitation of GPCR-targeted drugs is a patient's ability to gain tolerance or resistance to such drugs. This tolerance is attributed to the fact that GPCRs desensitize (i.e., turn off) their G protein signaling pathways in response to constant drug exposure.

One possible approach to overcoming GPCR-based drug tolerance is to inhibit GPCR desensitization with compositions having GPCR desensitization inhibitory activity. Because several hundred human GPCRs are known, and because it is estimated that a couple thousand GPCRs exist in the human genome, it would be desirable to provide a method of screening compositions for inhibitory effect on GPCR desensitization that is not receptor specific.

### SUMMARY OF THE INVENTION

The methods of the present invention involve screening a test composition for an indication of GPCR desensitization inhibitory activity against two or more GPCRs that are different from each other. When there is an indication that a particular test composition has GPCR desensitization inhibitory activity with respect to each of the two or more GPCRs that are different from one another, then, according to the present invention, there is an indication that the test composition has non-receptor-specific GPCR desensitization inhibitory activity.

In one aspect, a method is provided of screening a composition for non-receptor-specific GPCR desensitization inhibitory activity using two different cells. A first cell is provided comprising a first GPCR and a first conjugate of a marker molecule and a protein associated with the desensitization pathway of the first GPCR. The first GPCR is a GPCR that requires agonist for desensitization or is a constitutively desensitized GPCR. The first cell is exposed to a test composition and, when the first GPCR requires agonist for desensitization, to an agonist for the first GPCR. A determination is made, through detection of the marker molecule in the first conjugate, whether or not the composition gives an indication of GPCR desensitization inhibitory activity with respect to the first GPCR. A second cell is provided comprising a second GPCR that is different from the first GPCR and a second conjugate of a marker molecule and a protein associated with the desensitization pathway of the second GPCR. The second GPCR is a GPCR that requires agonist for desensitization or is a constitutively desensitized GPCR. The second conjugate may be the same or different from the first conjugate. The second cell is exposed to the test composition and, when the second GPCR requires agonist for desensitization, to an agonist for the second GPCR. A determination is made, through detection of the marker molecule in the second conjugate, whether or not the composition gives an indication of GPCR desensitization inhibitory activity with respect to the second GPCR. An indication of GPCR desensitization inhibitory activity for the test composition with respect to both the first and the second GPCRs is an indication that the test composition has non-receptor-specific GPCR desensitization inhibitory activity.

The determination of whether or not the composition gives an indication of GPCR desensitization inhibitory activity with respect to a GPCR can be made in various ways. For example, the determination could be made by detecting for translocation or localization of a conjugate (e.g., the first or the second conjugate) in a test cell, a lack of translocation or localization being an indication that the composition has GPCR desensitization inhibitory activity. As another example, the determination could be made by detecting for translocation or localization of a conjugate in a test cell, a decrease in translocation or localization after exposure to the test composition being an indication that the composition has GPCR desensitization inhibitory activity. As yet another example, the determination could be made by detecting for translocation or localization of a conjugate in a test cell, a decreased level of translocation or localization in the test cell with respect to a predetermined level or with respect to a level of translocation or localization determined in a control cell not exposed to the test composition being an indication that the composition has GPCR desensitization inhibitory activity. The control cell may comprise the same GPCR and conjugate used in the test cell and may be exposed to agonist if the GPCR requires agonist for desensitization.

In another aspect of the invention, a method is provided of screening a composition for non-receptor-specific G-protein coupled receptor (GPCR) desensitization inhibitory activity using one cell. A cell is provided comprising (1) a first GPCR that is a GPCR that requires agonist for desensitization; (2) a second GPCR that is different than the first GPCR, the second GPCR being a GPCR that requires agonist for desensitization; (3) a first conjugate of a marker molecule and a protein associated with the desensitization pathway of the first GPCR; and (4) a second conjugate of a marker molecule and a protein associated with the desensitization pathway of the second GPCR. The second conjugate may be the same or different from the first conjugate. The cell is exposed to a test composition and to an agonist for the first GPCR and a determination is made whether or not the composition has GPCR desensitization inhibitory activity with respect to the first GPCR. The cell is also exposed to an agonist for the second GPCR (and optionally re-exposed to the test composition) and a determination is made whether or not the composition has GPCR desensitization inhibitory activity with respect to the second GPCR. An indication that the test composition has GPCR desensitization inhibitory activity with respect to both the first GPCR and the second GPCR is an indication that the test composition has non-receptor-specific GPCR desensitization inhibitory activity. In the method, the agonist for the first GPCR is not an agonist for the second GPCR and the agonist for the second GPCR is not an agonist for the first GPCR. As above, the determination of whether or not the composition gives an indication of GPCR desensitization inhibitory activity with respect to a GPCR can be made in various ways.

In yet another embodiment of the invention, another method of screening a composition for non-receptor-specific G-protein coupled receptor (GPCR) desensitization inhibitory activity using one cell is provided. A cell is provided comprising (1) a first GPCR that is a GPCR that requires agonist for desensitization or is a constitutively desensitized GPCR, (2) a second GPCR that is different than the first GPCR, the second GPCR being a GPCR that requires agonist for desensitization or being a constitutively desensitized GPCR. (3) a first conjugate of a first marker molecule and a protein associated with the desensitization pathway of the first GPCR, and (4) a second conjugate of a second marker molecule and a protein associated with the desensitization pathway of the second GPCR, the second conjugate being different from the first conjugate. The protein of the first conjugate is not included in the desensitization pathway of the second GPCR and the protein in the second conjugate is not included in the desensitization pathway of the first GPCR. In addition, the first and second marker molecules are different from each other and are distinguishable from each other upon detection. The cell is exposed (I) to a test composition, (2) when the first GPCR requires agonist for desensitization, to an agonist for the first GPCR, and (3) when the second GPCR requires agonist for desensitization, to an agonist for the second GPCR and a determination is made whether or not the composition has GPCR desensitization inhibitory activity with respect to the first GPCR and with respect to the second GPCR. An indication that the test composition has GPCR desensitization inhibitory activity with respect to both the first GPCR and the second GPCR being an indication that the test composition has non-receptor-specific GPCR desensitization inhibitory activity. As above, the determination of whether or not the composition gives an indication of GPCR desensitization inhibitory activity with respect to a GPCR can be made in various ways.

### BRIEF DESCRIPTION OF THE DRAWINGS

FIG. 1 illustrates an example of a desensitization pathway of a GPCR in response to an agonist. Reference numerals in FIG. 1 correspond to items depicted therein as follows: cell membrane-1; GPCR or GPCR-marker molecule conjugate-2; carboxyl terminal tail of GPCR-3; extracellular region-4; intracellular region/cytosol-5; arrestin protein or arrestin-marker molecule conjugate-6; GPCR-arrestin protein complex-7; clathrin-coated pit/vesicle-8; endosome-9; agonist for GPCR-10; third intracellular loop-11; intramembrane portion of GPCR-12; G protein-coupled receptor kinase (GRK)-15; G protein-20.

FIG. 2 is an illustrative, non-limiting list of known GPCRs that may be used with the present invention.

FIG. 3 is an illustrative, non-limiting list of known receptors, including the amino acid sequence for their carboxyl terminal tails (Sequence ID Nos.: 1-39) and appropriate classification. For the Class B receptor examples, the residues that may function as phosphorylation sites in the enhanced affinity motifs are shown in bolded italics.

FIG. 4 illustrates the amino acid sequences of the following GPCRs in which the DRY motif has been modified: Vasopressin V2 Receptor (V2R), Alpha-1B Adrenergic Receptor (α_{1B}-AR), and Angiotensin II Receptor, Type 1 (AT_{1A}R). The figure illustrates the amino acid sequences of the receptors with the following mutations: V2R R137H (Sequence ID No.: 40), α_{1B}-AR R143E (Sequence ID No.: 41), α_{1B}-AR R143A (Sequence ID No.: 42), α_{1B}-AR R143H (Sequence ID No.: 43), α_{1B}-AR R143N (Sequence ID No.: 44), and AT_{1A}R R126H (Sequence ID No.: 45). Amino acids that differ from the wild-type sequence are in bold and underlined.

FIG. 5 is a list of amino acid and nucleic acid sequences of the following GPCRs that have been modified to have enhanced affinity for arrestin: hGPR3-Enhanced receptor, hGPR6-Enhanced receptor, hGPR12-Enhanced receptor, hSREB3-Enhanced receptor, hSREB2-Enhanced receptor, hGPR8-Enhanced receptor, and hGPR22-Enhanced receptor. FIGS. 5A and 5B respectively illustrate the amino acid sequence (Sequence ID No.: 46) and the nucleic acid sequence (Sequence ID No.: 47) of the hGPR3-Enhanced receptor. FIGS. 5C and 5D respectively illustrate the amino acid sequence (Sequence ID No.: 48) and the nucleic acid sequence (Sequence ID No.: 49) of the hGPR6-Enhanced receptor. FIGS. 5E and 5F respectively illustrate the amino acid sequence (Sequence ID No.: 50) and the nucleic acid sequence (Sequence ID No.: 51) of the hGPR12-Enhanced receptor. FIGS. 5G and 5H respectively illustrate the amino acid sequence (Sequence ID No.: 52) and the nucleic acid sequence (Sequence ID No.: 53) of the hSREB3-Enhanced receptor. FIGS. 5I and 5J respectively illustrate the amino acid sequence (Sequence ID No.: 54) and the nucleic acid sequence (Sequence ID No.: 55) of the hSREB2-Enhanced receptor. FIGS. 5K and 5L respectively illustrate the amino acid sequence (Sequence ID No.: 56) and the nucleic acid sequence (Sequence ID No.: 57) of the hGPR8-Enhanced receptor. FIGS. 5M and 5N respectively illustrate the amino acid sequence (Sequence ID No.: 58) and the nucleic acid sequence (Sequence ID No.: 59) of the hGPR22-Enhanced receptor.

FIG. 6 lists GPCRs that have been modified to have enhanced affinity for arrestin. FIG. 6A shows the amino acid sequence, termed SEQ ID NO.: 60, of the β₂AR-V2R chimera. FIG. 6B shows the amino acid sequence, termed SEQ ID NO.: 61, of the MOR-V2R chimera. FIG. 6C shows the amino acid sequence, termed SEQ ID NO.: 62, of the D1AR-V2R chimera. FIG. 6D shows the amino acid sequence, termed SEQ ID NO.: 63, of the 5HT1AR-V2R chimera. FIG. 6E shows the amino acid sequence, termed SEQ ID NO.: 64, of the β3AR-V2R chimera. FIG. 6F shows the amino acid sequence, termed SEQ ID NO.: 65, of the Edg1R-V2R chimera.

FIGS. 7-22 illustrate concentration-response plots of the average amount of fluorescent intensity of identified “grains” of arrestin-GFP localization (i.e., Fgrains) in cells expressing β₂AR and cells expressing V2R after addition of the indicated concentrations of test compound (or control compound) and addition of agonist. Each compound was tested against each receptor at room temperature and atmospheric CO₂ (indicated in the figures as O₂AR RT and V2R RT) and at 37° C. and 5% CO₂/95% O₂ (indicated in the figures as β2AR CO₂ and V2R CO₂). Concentration-response curves were plotted for the assays that indicated GPCR desensitization inhibitory activity of a compound. FIGS. 7-21 illustrate concentration-response plots for test compositions and FIG. 22 illustrates the concentration-response plot of a control compound as described in the Example below.

### DETAILED DESCRIPTION OF THE INVENTION

The present invention relates to methods of screening compositions for GPCR desensitization inhibitory activity that is not specific to a particular receptor. Prior to describing this invention in further detail, however, the following terms will first be defined.

Definitions:

“Arrestin” means all types of naturally occurring and engineered variants of arrestin, including, but not limited to, visual arrestin (sometimes referred to as Arrestin 1), cone arrestin (sometimes referred to as arrestin-4), β-arrestin 1 (sometimes referred to as Arrestin 2), and β-arrestin 2 (sometimes referred to as Arrestin 3). “Arrestin” also includes biologically active fragments of arrestin.

“Biologically active fragment” of an arrestin means a fragment of arrestin that has the ability to bind a wild-type and/or modified GPCR.

An “antibody” is any immunoglobulin, including antibodies and fragments thereof, that bind a specific epitope.

“Carboxyl-terminal tail” means the carboxyl-terminal tail of a GPCR following membrane span 7. The carboxyl-terminal tail of many GPCRs begins shortly after the conserved NPXXY motif that marks the end of the seventh transmembrane domain (i.e. what follows the NPXXY motif is the carboxyl-terminal tail of the GPCR). The carboxyl-terminal tail may be relatively long (approximately tens to hundreds of amino acids), relatively short (approximately tens of amino acids), or virtually non-existent (less than approximately ten amino acids). As used herein, “carboxyl-terminal tail” shall mean all three variants (whether relatively long, relatively short, or virtually non-existent), and may or may not contain palmitoylated cysteine residue(s).

“Marker molecule” means any molecule capable of detection by spectroscopic, photochemical, biochemical, immunochemical, electrical, radioactive, and optical means, including but not limited to, fluorescence, phosphorescence, and bioluminescence and radioactive decay. Marker molecules include, but are not limited to, GFP, luciferase, β-galactosidase, rhodamine-conjugated antibody, and the like. Marker molecules include radioisotopes, epitope tags, affinity labels, enzymes, fluorescent groups, chemiluminescent groups, and the like. Marker molecules include molecules that are directly or indirectly detected as a function of their interaction with other molecule(s).

“GFP” means Green Fluorescent Protein, which refers to various naturally occurring forms of GFP that may be isolated from natural sources or genetically engineered, as well as artificially modified GFPs. GFPs are well known in the art. See, for example, U.S. Pat. Nos. 5,625,048; 5,777,079; and 6,066,476. It is well understood in the art that GFP is readily interchangeable with other fluorescent proteins, isolated from natural sources or genetically engineered, including but not limited to, yellow fluorescent proteins (YFP), red fluorescent proteins (RFP), cyan fluorescent proteins (CFP), blue fluorescent proteins, luciferin, UV excitable fluorescent proteins, or any wave-length in between. As used herein, “GFP” shall mean all fluorescent proteins known in the art.

“Downstream” means toward a carboxyl-terminus of an amino acid sequence, with respect to the amino-terminus.

“Upstream” means toward an amino-terminus of an amino acid sequence, with respect to the carboxyl-terminus.

“GPCR” means G protein-coupled receptor and includes GPCRs naturally occurring in nature, as well as GPCRs that have been modified, including the GPCRs described in U.S. patent application Ser. Nos. 09/993,844 and 10/054,616 and the GPCRs described in U.S. Provisional Patent Application No. 60/401,698.

“Desensitized GPCR” means a GPCR that presently does not have ability to respond to agonist and activate conventional G protein signaling.

“Sensitized GPCR” means a GPCR that presently has ability to respond to agonist and activate conventional G protein signaling.

“GPCR desensitization pathway” means any cellular component of the GPCR desensitization process, as well as any cellular structure implicated in the GPCR desensitization process and subsequent processes, including but not limited to, arresting, GRKs, GPCRs, AP-2 protein, clathrin, protein phosphatases, and the like.

“GPCR signaling” means GPCR induced activation of G proteins. This may result in, for example, cAMP production.

“G protein-coupled receptor kinase” (GRK) includes any kinase that has the ability to phosphorylate a GPCR.

An “overexpressed” protein refers to a protein that is expressed at levels greater than wild-type expression levels.

“Restriction enzymes” refers to bacterial enzymes, each of which cut double-stranded DNA at or near a specific nucleotide sequence.

“Unknown Receptor” or “Orphan Receptor” means a GPCR whose endogenous ligand(s) is/are unknown.

A “vector” is a replicon, such as plasmid, phage or cosmid, to which another DNA segment may be attached so as to bring about the replication of the attached segment.

“GPCR desensitization inhibitory activity” of a composition (e.g., compound, solution, etc.) means that the composition is capable of inhibiting GPCR desensitization of at least one specific GPCR.

“Non-receptor-specific GPCR desensitization inhibitory activity” of a composition (e.g., compound, solution, etc.) means that the composition is capable of inhibiting GPCR desensitization of two or more specific GPCRs. In some embodiments, compositions with “non-receptor-specific GPCR desensitization inhibitory activity” may be capable of inhibiting GPCR desensitization in all or a portion of the GPCRs in a specified class (e.g., class A, class B, class I, class II, class III, etc.) and/or a specified family (e.g., serotonin, opioid, adenosine, adrenergic, dopamine, GABA, etc.). Compositions with “non-receptor-specific GPCR desensitization inhibitory activity” may inhibit GPCR desensitization by affecting, either directly or indirectly, one or more cellular components of the GPCR desensitization pathway other than the GPCR itself such as, for example, by inhibiting a GRK or an arrestin protein.

An “indication” of GPCR desensitization inhibitory activity or non-receptor-specific GPCR desensitization inhibitory activity means something (e.g., an event, sign, signal, etc. or the lack thereof) that indicates evidence of such activity.

“Test composition” or “composition” means any solution, compound, or other substance (including, but not limited to, small molecules such as deoxyribonucleotide (DNA) and ribonucleotide (RNA) molecules as well as peptides and proteins) to be screened according to the present invention for non-receptor-specific GPCR desensitization inhibitory activity.

“Desensitization” or “GPCR desensitization” refers generally to the process by which sensitized GPCRs are converted to desensitized GPCRs.

Methods of Screening Compositions

The methods of the present invention allow the screening of a test composition for non-receptor-specific GPCR desensitization inhibitory activity. The methods involve screening a test composition for an indication of GPCR desensitization inhibitory activity against two or more GPCRs that are different from each other. When there is an indication that a particular test composition has GPCR desensitization inhibitory activity with respect to each of the two or more GPCRs that are different from one another, then, according to the present invention, there is an indication that the test composition has non-receptor-specific GPCR desensitization inhibitory activity. Detection methods for determining whether there is an indication that a test composition has GPCR desensitization inhibitory activity are discussed below.

The methods of the present invention may be conducted using one or more cells. In one embodiment, two or more cells are used to screen a test composition for non-receptor-specific GPCR desensitization inhibitory activity. In such an embodiment, each cell expresses at least one GPCR that is different from the GPCR or GPCRs being used to screen the test composition in the other cell or cells. In such an embodiment, each cell must express (or overexpress) a GPCR such that a detection method may be used for determining whether there is an indication that a test composition has GPCR desensitization inhibitory activity with respect to that specific GPCR. The GPCR used in one of the cells to screen a test composition may be absent in the other cells that use different GPCRs for screening, may be present in the other cells that use different GPCRs for screening at a level that does not affect the screening using the different GPCRs, or may be present in the other cells that use different GPCRs for screening at the same level (or a higher or lower level) as in the cell that is using the GPCR for screening when an appropriate detection method is used that may determine an indication of GPCR desensitization inhibitory activity with respect to the appropriate GPCR.

In another embodiment, one cell is used to screen a test composition for non-receptor-specific GPCR desensitization inhibitory activity. In such an embodiment, the cell expresses (or overexpresses) two or more GPCRs that are different from each other such that a detection method may be used for determining whether there is an indication that a test composition has GPCR desensitization inhibitory activity with respect to each of the different GPCRs. As used herein, an embodiment using one cell to screen a test composition for non-receptor-specific GPCR desensitization includes using only one cell as well as using multiple cells expressing the same two or more GPCRs, and details concerning such an embodiment (i.e., using one cell) are not meant to limit the embodiment to using only one cell.

Each cell used in the methods also includes one or more conjugates comprising a marker molecule and a protein associated with the GPCR desensitization pathway of one or more of the GPCRs that are being used in the cell to screen a test composition for GPCR desensitization inhibitory activity. The conjugate or conjugates indicate, through the use of the marker molecule, GPCR desensitization inhibitory activity of a test composition with respect to each of the GPCRs that are being used to screen the test composition. The conjugates of the one or more cells may comprise, for example, an arrestin protein and a marker molecule and/or a GPCR and a marker molecule. In one embodiment, the cell or cells may comprise a conjugate of an arrestin protein and a marker molecule as well as a conjugate of a GPCR and a marker molecule.

When one cell is used to screen a test composition for non-receptor-specific GPCR desensitization inhibitory activity, the cell expresses two or more GPCRs that are different from one another (e.g., a first GPCR and a second GPCR that is different from the first GPCR). The cell also includes one or more conjugates comprising a marker molecule and a protein associated with the GPCR desensitization pathway of one or more of the GPCRs expressed in the cell. The cell will include one or more appropriate conjugates such that, through the use of the marker molecule(s) of the conjugate(s), a detection method or methods can be used to determine whether there is an indication of GPCR desensitization inhibitory activity of a test composition with respect to each GPCR used for screening in the cell.

Methods using one cell to screen a test composition for non-receptor-specific GPCR desensitization inhibitory activity involve using at least a first GPCR and a second GPCR that is different than the first GPCR. Each cell also includes a first conjugate of a marker molecule and a protein associated with the desensitization pathway of the first GPCR and a second conjugate of a marker molecule and a protein associated with the desensitization pathway of the second GPCR. The marker molecules and/or the proteins associated with the desensitization pathways may be the same or different in the first and second conjugates. When both the marker molecule and the protein associated with the desensitization pathway are the same in the first and second conjugates, it is possible to use only one type of conjugate in the cell. It should be noted, however, that other embodiments could use multiple conjugates containing different marker molecules and/or proteins associated with the desensitization pathways of different GPCRs that are being used to screen a test composition. In such embodiments, different conjugates could be used to indicate desensitization inhibitory activity of a composition with respect to different GPCRs being used to screen a test composition. Also in such embodiments, multiple conjugates could be used (in combination or separately) to indicate GPCR desensitization inhibitory activity of a composition with respect to the same GPCR. In methods using one cell, an indication of non-receptor-specific GPCR desensitization inhibitory activity is shown with respect to a test composition when there is an indication that the test composition has GPCR desensitization inhibitory activity with respect to each of the two or more GPCRs in the cell.

When two or more cells are used to screen a test composition for non-receptor-specific GPCR desensitization inhibitory activity, each cell expresses at least one GPCR that is different from the GPCR or GPCRs being used to screen the test composition in the other cell or cells. Each cell also includes one or more conjugates comprising a marker molecule and a protein associated with the GPCR desensitization pathway of one or more of the GPCRs expressed in the cell being used to screen a test composition. The conjugates may be the same or different in each of the two or more cells. As stated above, the conjugate or conjugates indicate, through the use of the marker molecule, GPCR desensitization inhibitory activity of a test composition.

Methods using two (or more) cells involve using a first cell having a first GPCR and a second cell having a second GPCR that is different from the first GPCR. The first cell includes a first conjugate of a protein associated with the GPCR desensitization pathway of the first GPCR (e.g., an arrestin protein, the first GPCR, etc.) and a marker molecule, and the second cell includes a second conjugate of a protein associated with the GPCR desensitization pathway of the second GPCR and a marker molecule. The second conjugate may be the same or different from the first conjugate. That is, the protein associated with the GPCR desensitization pathway may be the same or different in the first conjugate and the second conjugate. The marker molecule may also be the same or different in the first conjugate and the second conjugate. In addition, more than one conjugate may be included in each cell and more than one GPCR may be expressed in each cell to be used to screen a test composition. In methods using two or more cells (where each cell has one or more GPCRs that are different from the GPCR(s) in the other cell or cells), an indication of non-receptor-specific GPCR desensitization inhibitory activity is shown with respect to a test composition when there is an indication that the test composition has GPCR desensitization inhibitory activity with respect to each of the two or more different GPCRs.

As discussed above, an indication that a test composition has non-receptor-specific GPCR desensitization inhibitory activity is shown when there is an indication that the particular test composition has GPCR desensitization inhibitory activity with respect to each of the two or more GPCRs that are different from one another being used to screen the test composition. The indication of non-receptor-specific GPCR desensitization inhibitory activity may be shown with respect to two or more GPCRs that are different from one another that are expressed in one or more cells. In order to determine whether there is an indication that a test composition has non-receptor-specific GPCR desensitization inhibitory activity, the test composition is screened for GPCR desensitization inhibitory activity with respect to each of the two or more GPCRs being used in a specific embodiment.

In order to screen a test composition for GPCR desensitization inhibitory activity in a cell with respect to at least one specific GPCR, the cell expressing the GPCR is placed or held under conditions necessary for GPCR desensitization to occur of at least that specific GPCR and the cell is also exposed to the test composition. The conditions necessary for GPCR desensitization to occur in a cell may vary from cell to cell and from GPCR to GPCR. For example, desensitization of some GPCRs is agonist-dependent, while desensitization of some modified GPCRs (described more fully below) occurs constitutively in an agonist-independent manner. In addition, those GPCRs in which desensitization occurs in an agonist-dependent manner may require different agonists for desensitization depending upon the particular GPCR.

When the GPCR that is being used to screen for GPCR desensitization activity of a test composition is a GPCR that requires agonist for desensitization, the cell expressing the GPCR is exposed to a test composition and to an agonist for the GPCR, either simultaneously or serially in any order. When exposing a cell to a test composition and to an agonist serially, the sequence and the timing in between exposure to the test composition and to the agonist will depend upon the specific embodiment of the present invention being used. A detection method is used to detect for an indication of GPCR desensitization inhibitory activity of the test composition. If the GPCR used to screen for GPCR desensitization activity of a test composition is a modified GPCR that does not require agonist for GPCR desensitization, the cell expressing the GPCR is exposed to a test composition and a suitable detection method is used to detect for an indication of GPCR desensitization inhibitory activity of the test composition. Detection methods useful in the present invention are explained more fully below.

In embodiments using one cell expressing two or more different GPCRs, various formats could be used for screening a composition for non-receptor-specific GPCR desensitization activity. In such embodiments, the methods will generally comprise exposing the cell to a test composition, to an agonist for the first GPCR (when the first GPCR requires agonist for desensitization), and to an agonist for the second GPCR (when the second GPCR requires agonist for desensitization) and determining whether or not the composition has GPCR desensitization inhibitory activity with respect to the first GPCR and with respect to the second GPCR. The specific order of exposing the cell to the test composition and one or more agonists (when needed) may vary based upon various factors such as the desensitization pathway of each GPCR and the conjugate or conjugates used in the cell. In addition, measures may be taken such that indications of GPCR desensitization inhibitory activity with respect to different GPCRs in the cell may be distinguished. For example, a time separation could be used between the screening of a test composition with respect to different GPCRs (e.g., between screening with respect to a first GPCR and screening with respect to a second GPCR) such that indications of GPCR desensitization inhibitory activity of a composition with respect to the different GPCRs may be distinguished. As another example, separate conjugates could be used for the screening of a test composition with respect to each GPCR used in the cell such that each conjugate is included only in the desensitization pathway of one of the GPCRs and such that each conjugate contains different marker molecules that are distinguishable from each other upon detection.

In some embodiments of methods for screening for non-receptor-specific GPCR desensitization inhibitory activity of a test composition using one cell, two or more different GPCRs that require agonist for desensitization or are constitutively desensitized could be used. In general, such methods will comprise exposing the cell to a test composition, to an agonist for the first GPCR (when the first GPCR requires agonist for desensitization), and to an agonist for the second GPCR (when the second GPCR requires agonist for desensitization) and then determining whether or not the composition has GPCR desensitization inhibitory activity with respect to the first GPCR and with respect to the second GPCR. In such embodiments, an indication of GPCR desensitization inhibitory activity with respect to the first GPCR and an indication of GPCR desensitization inhibitory activity with respect to the second GPCR may be distinguished by using a different conjugate for the determination of GPCR desensitization inhibitory activity of the compositions with respect to the different GPCRs. That is, conjugates may be chosen such that the protein of the first conjugate is not included in the desensitization pathway of the second GPCR, the protein of the second conjugate is not included in the desensitization pathway of the first GPCR, and the first and second conjugates contain different marker molecules that are distinguishable from each other upon detection. As an example, a cell could include a first conjugate comprising a first GPCR and a first marker molecule and a second conjugate comprising a second GPCR and a second marker molecule that is different and distinguishable from the first marker molecule. In such an embodiment, it may be possible to expose the cell to the test composition, the agonist for the first GPCR (if needed for desensitization), and the agonist for the second GPCR (if needed for desensitization) simultaneously or non-simultaneously and determine whether the composition has GPCR desensitization inhibitory activity with respect to the first GPCR and the second GPCR.

In other embodiments of methods for screening for non-receptor-specific GPCR desensitization inhibitory activity of a test composition using one cell with two or more GPCRs requiring an agonist for desensitization, a time separation may be used between screening each of the GPCRs in the cell. For example, a cell comprising a first GPCR and a second GPCR could be exposed to an agonist for the first GPCR and then, after a sufficient amount of time, the cell could be exposed to an agonist for the second GPCR such that indications of desensitization inhibitory activity with respect to the first and second GPCRs may be sufficiently distinguished. In such an embodiment, the cell could be exposed to the test composition at various times before, after, or during exposure to the agonist for the first GPCR and the cell could be re-exposed to the test composition before, during, or after exposure to the agonist for the second GPCR. In addition, in such an embodiment, the GPCRs could be chosen such that the agonist for the first GPCR is not an agonist for the second GPCR and the agonist for the second GPCR is not an agonist for the first GPCR such that indications of desensitization inhibitory activity of the test compound could be distinguished with respect to the first GPCR and the second GPCR.

As mentioned above, the marker molecule(s) of the conjugate(s) in each cell is/are used to provide an indication of whether a test composition has GPCR desensitization inhibitory activity in that particular cell with respect to the GPCR or GPCRs being used in that cell to screen the test composition. Based upon the GPCR desensitization pathway, various formats of detection methods may be used as an indication that a test composition has GPCR desensitization inhibitory activity. The format that is used will depend somewhat on the particular protein associated with the desensitization pathway to which the marker molecule is conjugated.

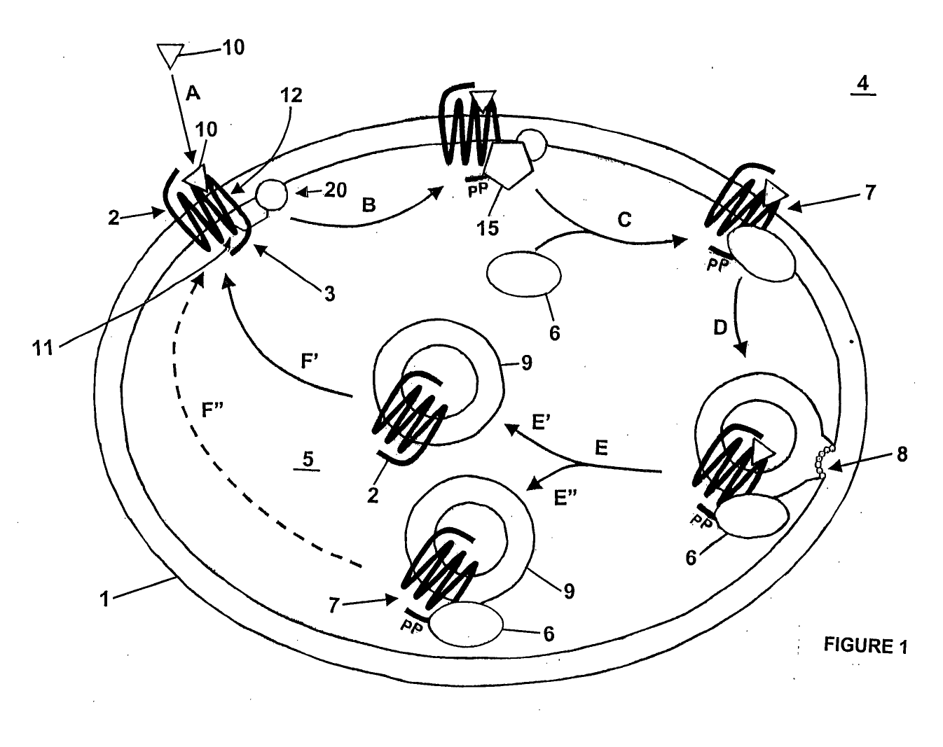

For example, by referring to and describing FIG. 1 (which illustrates an example of a desensitization pathway of a GPCR in response to an agonist), formats of detection methods using conjugates of an arrestin protein and a marker molecule and/or a GPCR and a marker molecule will be better understood. With reference to FIG. 1, after an agonist 10 interacts with a GPCR 2 to activate the GPCR 2 (shown by arrow A), one or more GRKs 15 phosphorylate clusters of serine and threonine residues located in the third intracellular loop 11 or the carboxyl-terminal tail 3 of the GPCR 2 (shown by arrow B). After phosphorylation, an arrestin protein 6 associates with the GRK-phosphorylated GPCR 2 and uncouples the GPCR 2 from its cognate G protein 20 to terminate GPCR signaling and produce a desensitized GPCR. Translocation of the arrestin 6 to the GPCR 2 is shown by arrow C. After the arrestin 6 binds to the GPCR 2, the arrestin/GPCR complex 7 targets to clathrin-coated pits or vesicles 8 (shown by arrow D) for endocytosis. Internalization of the GPCR 2 alone or the arrestin/GPCR complex 7 into endosomes 9 is shown by arrow E. Arrow E′ shows internalization of the GPCR 2 into an endosome 9. Arrow E″ shows internalization of the arrestin/GPCR complex 7 into an endosome 9. After or during internalization, the GPCR 2 is dephosphorylated and is recycled back to the cell membrane 1 as a resensitized GPCR 2. Recycling of the GPCR 2 that was internalized alone is shown by arrow F′. Recycling of the GPCR 2 that was internalized as the arrestin/GPCR complex 7 is shown by arrow F″.

With reference to FIG. 1, when a conjugate of an arrestin protein and a marker molecule is used in a cell, the detection method could detect for any of the following, the lack of which would be an indication that the test composition has GPCR desensitization inhibitory activity: (1) translocation of the arrestin conjugate 6 from the cytosol 5 to the cell membrane 1 (i.e., arrow C); (2) localization of the arrestin conjugate 6 at the plasma membrane 1; (3) translocation of the arrestin conjugate 6 from the cell membrane 1 to clathrin coated pits/vesicles 8, endosomes 9, or the cytosol 5 (i.e., arrows D and E); or (4) localization of the arrestin conjugate 6 at clathrin coated pits/vesicles 8, endosomes 9, or the cytosol 5. As another example, when a conjugate of a GPCR and a marker molecule is used in a cell, the detection method could look for any of the following, the lack of which would be an indication that the test composition has GPCR desensitization inhibitory activity: (1) translocation of the GPCR conjugate 2 from the cell membrane 1 to clathrin coated pits/vesicles 8, endosomes 9, or the cytosol 5 (i.e., arrows D and E); or (2) localization of the GPCR conjugate 2 at clathrin coated pits/vesicles 8, endosomes 9, or the cytosol 5. As yet another example, when both a conjugate of an arrestin protein and a marker molecule and a conjugate of a GPCR and a marker molecule are used in a cell, the detection method could look for any of the items/events listed above as well as for localization of the arrestin conjugate with the GPCR conjugate, the lack of which would be an indication that the test composition has GPCR desensitization inhibitory activity. Although the formats of detection methods above are described with respect to FIG. 1, which includes addition of an agonist for GPCR desensitization, these formats are not meant to be limited to the use of an agonist, and also apply to other embodiments such as, for example, embodiments using one or more GPCRs that are constitutively desensitized.

Detection for each of the items/events discussed above could be conducted at one point in time, over a period of time, at two or more points in time for comparison (e.g., before and after exposure to a test composition), etc. An indication of GPCR desensitization inhibitory activity could be determined by detecting for one or more of the items/events discussed above in a cell exposed to the test composition and comparing the results to those obtained by detecting for the same item(s)/event(s) in a control cell, by comparing the results to a predetermined value, or without reference to a predetermined level or a control cell. Therefore, in addition to using a lack of certain items/events as indications of GPCR desensitization inhibitory activity, a decrease in the level of any of the same items/events discussed above after exposure to a test composition could be used as an indication of GPCR desensitization activity of the test composition. Detecting for a decrease in the level of the items/events discussed above (e.g., as compared to a control cell not being exposed to the test composition, as compared to a predetermined level, or as compared to a level before exposure to the test composition) may be useful in embodiments where a test composition is added when desensitization is already occurring such as, for example, in embodiments using a cell expressing a constitutively desensitized GPCR or in embodiments where a test composition is added after the cell has been exposed to an agonist.

G-Protein Coupled Receptors (GPCRs)

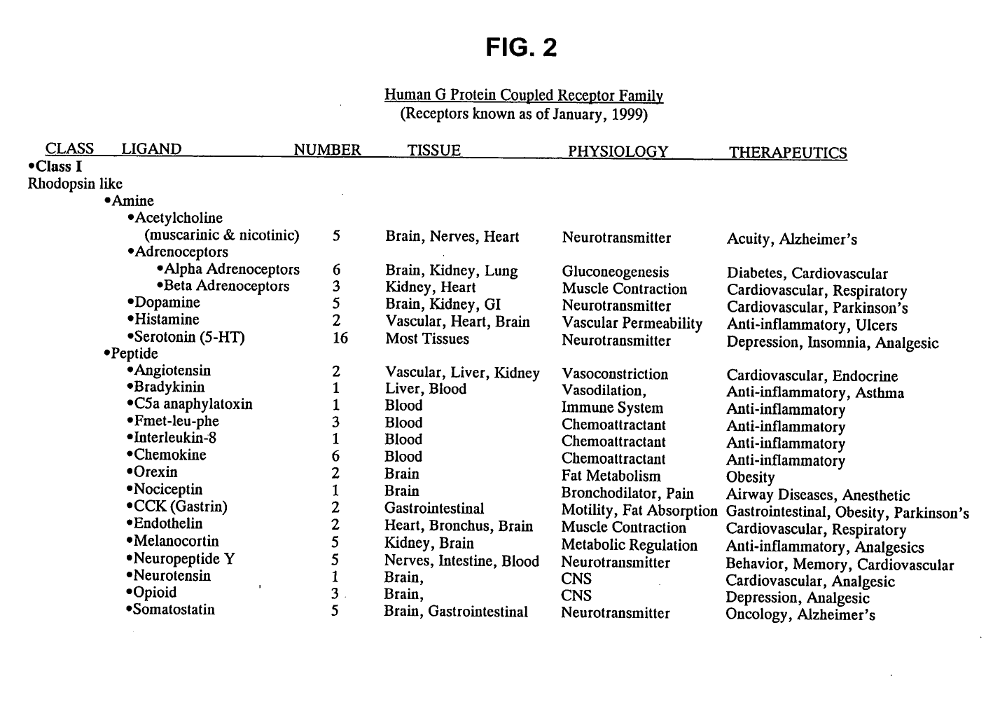

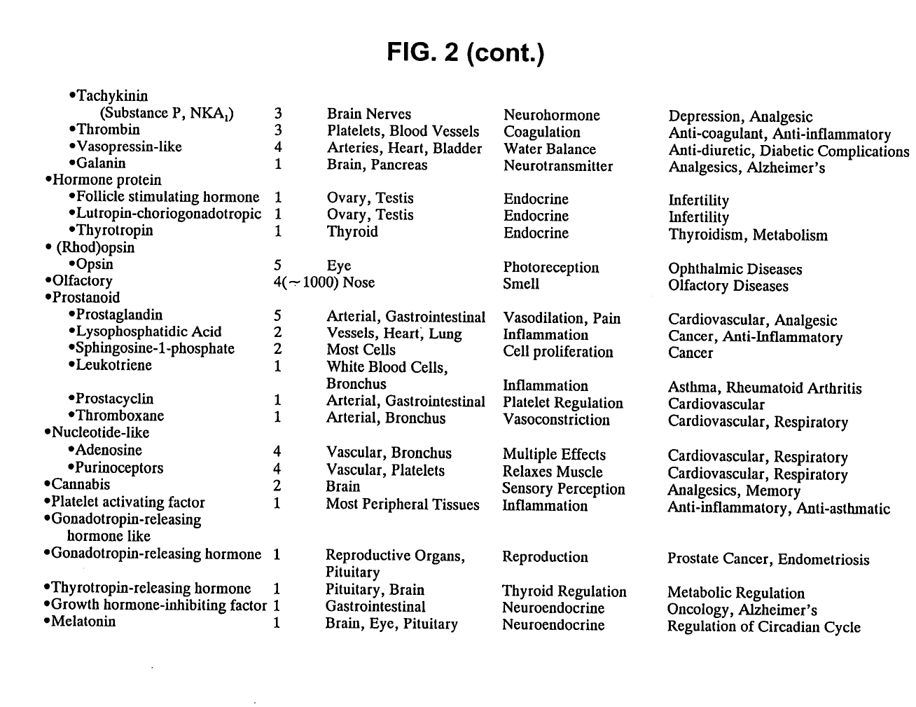

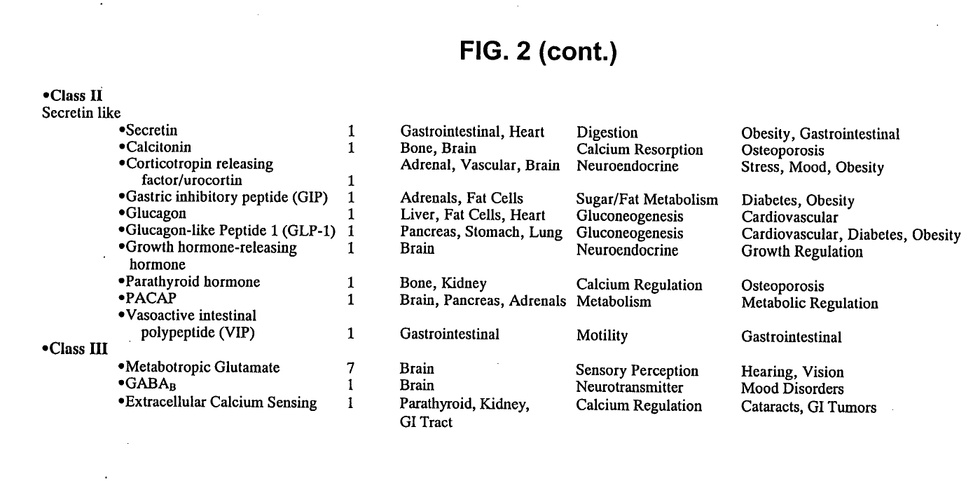

Any G-protein coupled receptor (GPCR) may be used in the methods of the present invention that is capable of participating in the GPCR desensitization pathway such that GPCR desensitization inhibitory activity of a test composition may be determined. An illustrative, non-limiting list of known GPCRs with which the present invention may be used is contained in FIG. 2. The receptors are grouped according to classical divisions based on structural similarities and ligands. GPCRs that may be used in the present invention include known GPCRs, unknown or orphan GPCRs, and chimeric or modified GPCRs (described more fully below). Modified GPCRs include GPCRs that have one or more modifications in the carboxyl-terminal tail, modifications in the intracellular loop(s), and/or in the cytoplasmic end of the transmembrane region.

By way of example, three major classes of GPCRs for known receptors have been identified: Class A receptors, Class B receptors, and receptors with virtually non-existent carboxyl-terminal tails. The receptors are classified accordingly based on their interactions with an affinity for rat β-arrestin-2 in HEK-293 cells and may be predicted based on the amino acid residues in their carboxyl-terminal tail and the length of their carboxyl-terminal tail. A Class B receptor is a GPCR that has one or more sites of phosphorylation (e.g., clusters of phosphorylation sites) properly positioned in its carboxyl-terminal tail such that it does recruit rat β-arrestin-2 to endosomes in HEK-293 cells under conditions as described in U.S. Pat. No. 5,891,646, Oakley, et al. “Differential Affinities of Visual Arrestin, βArrestin1, and βArrestin2 for G Protein-coupled Receptors Delineate Two Major Classes of Receptors,” Journal of Biological Chemistry, Vol 275, No. 22, pp 17201-17210, Jun. 2, 2000, and Oakley et al., “Molecular Determinants Underlying the Formation of Stable Intracellular G Protein-coupled Receptor-β-Arrestin Complexes after Receptor Endocytosis,” Journal of Biological Chemistry, Vol. 276, No. 22, pp 19452-19460, 2001, the contents of which are hereby incorporated by reference in their entirety. A Class A receptor is a GPCR that does not have one or more sites of phosphorylation (e.g., clusters of phosphorylation sites) properly positioned in its carboxyl-terminal tail such that it does not recruit rat β-arrestin-2 to endosomes in HEK-293 cells under conditions as described above for Class B receptors. Receptors with virtually non-existent carboxyl-terminal tails include, for example, olfactory and taste receptors.

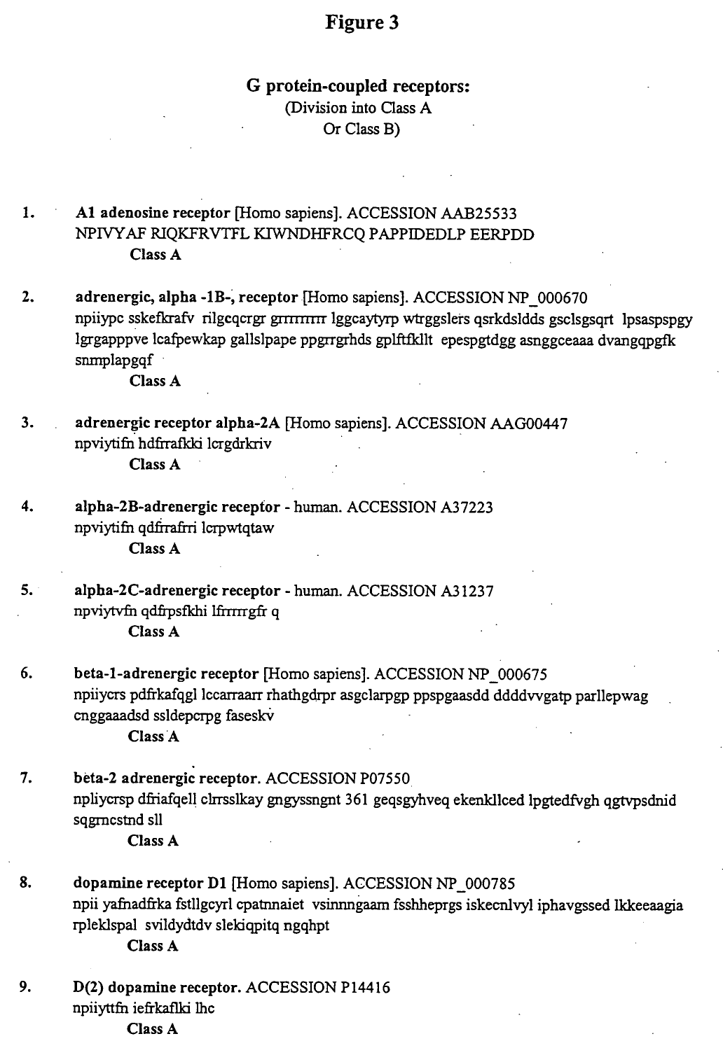

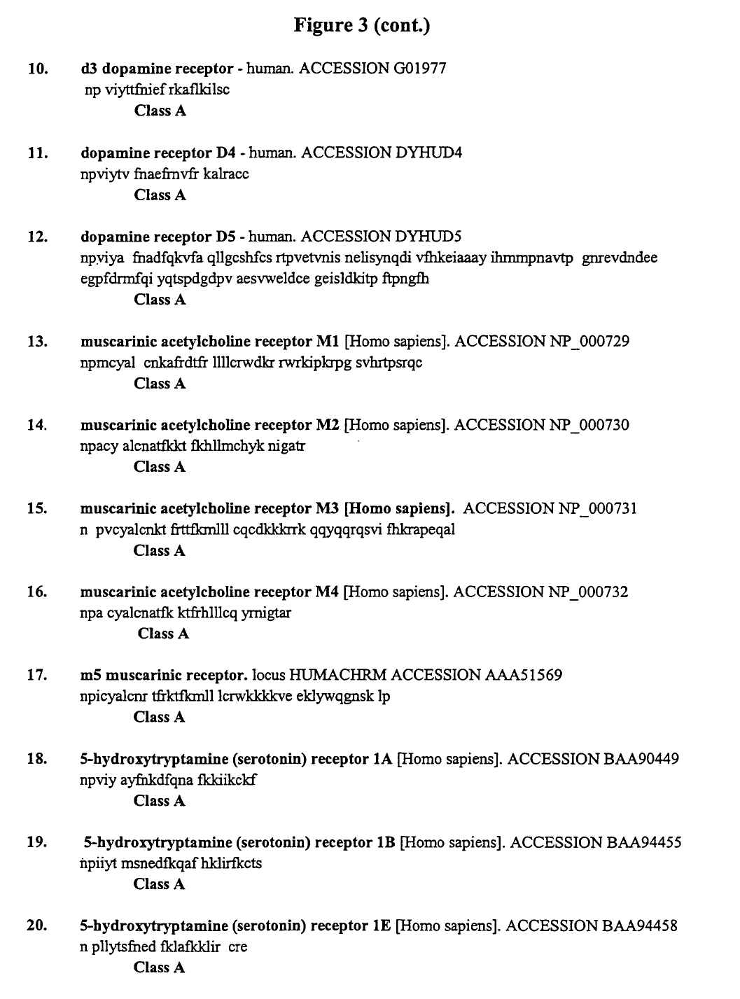

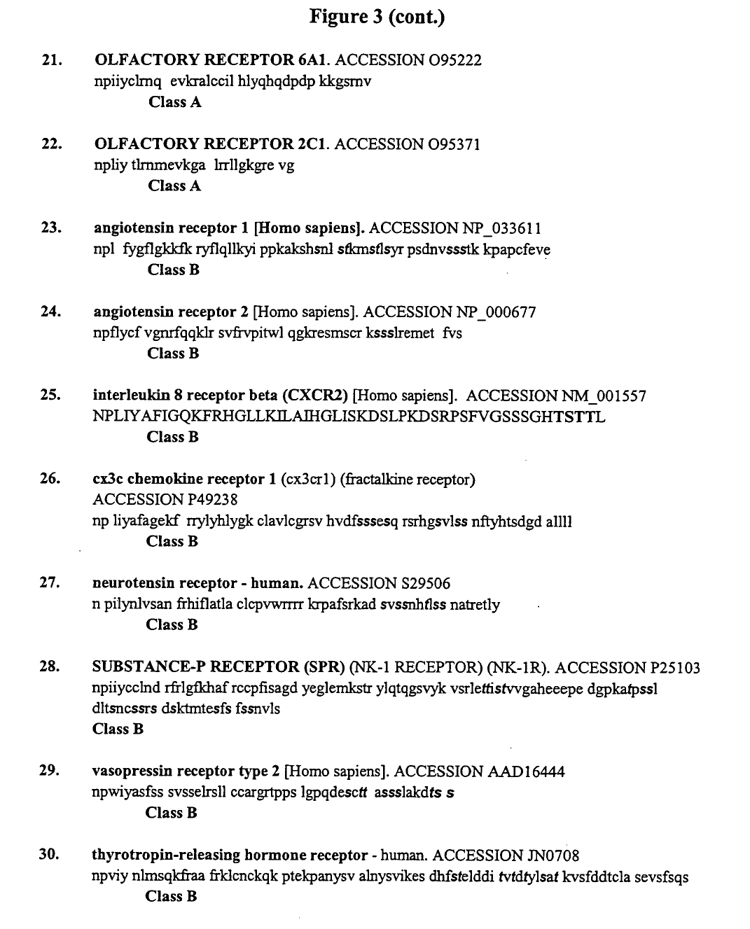

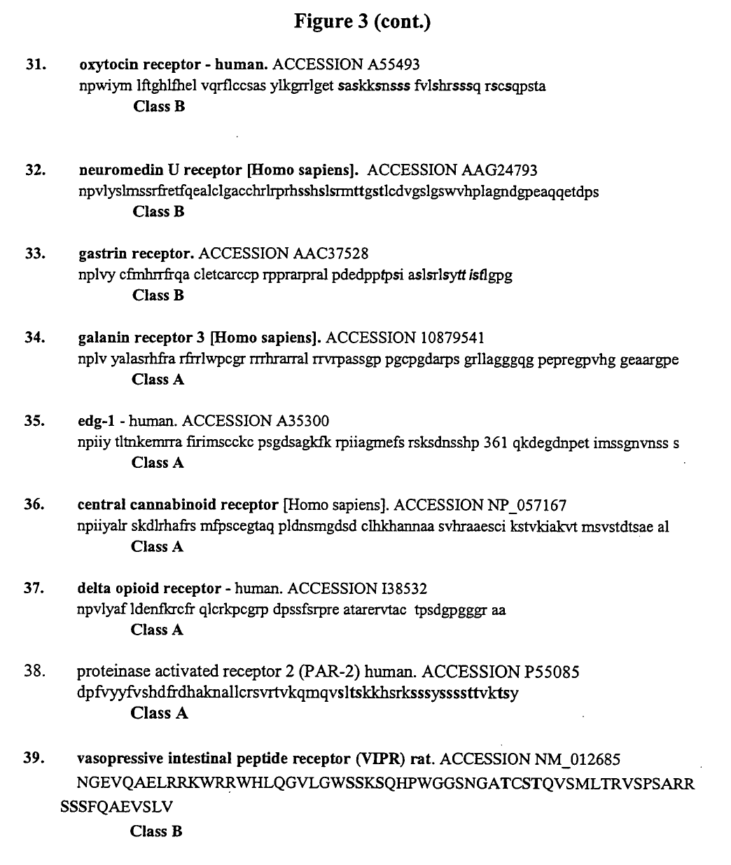

FIG. 3 is an illustrative, non-limiting list of known receptors, including the amino acid sequence for their carboxyl terminal tails and appropriate classification. For the Class B receptor examples, the residues that may function as clusters of phosphorylation sites are shown in bolded italics.

After agonists bind and activate GPCRs, G protein-coupled receptor kinases (GRKs) phosphorylate clusters of serine and threonine residues located in the third intracellular loop or the carboxyl-terminal tail of the GPCRs. After phosphorylation, an arrestin protein associates with the GRK-phosphorylated receptor and uncouples the receptor from its cognate G protein. The interaction of the arrestin with the phosphorylated GPCR terminates GPCR signaling and produces a non-signaling, desensitized receptor.

The arrestin bound to the desensitized GPCR targets the GPCR to clathrin-coated pits for endocytosis by functioning as an adaptor protein, which links the GPCR to components of the endocytic machinery, such as adaptor protein-2 (AP-2) and clathrin. The internalized GPCRs are dephosphorylated and are recycled back to the cell surface resensitized.

The stability of the interaction of arrestin with the GPCR dictates the rate of GPCR dephosphorylation, recycling, and resensitization. When the GPCR has an enhanced affinity for arrestin, the GPCR/arrestin complex is stable, remains intact and is internalized into endosomes. When the GPCR does not have an enhanced affinity for arrestin, the GPCR/arrestin complex tends not to be stable and arrestin is not recruited into endosomes with the GPCR. When the GPCR has an enhanced affinity for arrestin, the GPCR/arrestin complex remains intact, and the GPCR dephosphorylates, recycles and resensitizes slowly. In contrast, GPCRs that dissociate from arrestin at or near the plasma membrane dephosphorylate and recycle rapidly.

The ability of the arrestin to remain associated with the GPCRs is mediated by one or more sites of phosphorylation (e.g., clusters of phosphorylation sites) properly positioned within the carboxyl-terminal tail. These clusters of phosphorylation sites are may be serine and threonine residues located in the carboxyl-terminal tail of the GPCR. These clusters are remarkably conserved in their position within the carboxyl-terminal tail domain and serve as primary sites of agonist-dependent phosphorylation.

Modified GPCRs

1. Constitutively Desensitized GPCRs

The modified GPCRs of the present invention include GPCRs comprising a modified DRY motif as described in U.S. patent application Ser. No. 10/054,616, filed Jan. 22, 2002, the content of which is hereby incorporated by reference herein in its entirety. A DRY motif is a highly conserved GPCR motif located near the cytoplasmic boundary of the third transmembrane region and the second intracellular loop. The DRY motif is typically a three amino acid motif: Aspartate-Arginine-Tyrosine (DRY). A modified DRY motif refers to a DRY motif of a GPCR that has one or modifications resulting in an amino acid sequence other than the DRY motif. In one embodiment, the modified DRY motif consists of an amino acid other than arginine as the second amino acid. The second amino acid of the modified DRY motif is typically Alanine, Aspartate, Glutamate, Histidine, or Asparagine, but may be any amino acid other than arginine or Lysine. This modified DRY motif results in a constitutively desensitized GPCR. As described herein, the DRY motif of any GPCR can be modified as described, resulting in a constitutively desensitized GPCR. This modification may allow the modified GPCR to bind arrestin in the absence of agonist.

A wild-type GPCR cycles between being (1) sensitized, which means presently able to respond to agonist and activate conventional G protein signaling, (2) desensitized, which means presently unable to respond to agonist and activate conventional G protein signaling, and (3) resensitized, which means again presently able to respond to agonist and activate conventional G protein signaling. This balance can be disrupted, resulting, for example, in a constitutively desensitized GPCR. A constitutively desensitized GPCR does not cycle as above. Under wild-type conditions, a GPCR is desensitized subsequent to agonist activation of the sensitized GPCR; whereas, a constitutively desensitized GPCR forms independent of agonist stimulation of the sensitized GPCR. In a particular embodiment, the modified GPCRs of the present invention include GPCRs that have been modified so that they localize to endocytic vesicles or endosomes in an agonist-independent manner.

In a constitutively desensitized GPCR, the equilibrium between having the ability, versus inability, to properly activate conventional G protein signaling is shifted toward the inability to properly activate conventional G protein signaling. Additionally, the constitutively desensitized GPCRs of the present invention are constitutively phosphorylated, constitutively bind arrestin, constitutively localize in clathrin-coated pits, and/or constitutively localize to endocytic vesicles or endosomes. Constitutively desensitized receptors lack ability to properly respond to agonist and may be desensitized even in the absence of agonist. Constitutively desensitized GPCRs form independent of agonist stimulation of the sensitized GPCR. Constitutively desensitized GPCRs may include a host of degrees of inappropriate signaling and a constitutively desensitized receptor may or may not signal at some point during its lifetime.

The modified GPCRs of the present invention may comprise one or more modifications in the DRY motif. The DRY motif may be modified in one or more of the three amino acids, but must be modified at least in the second position (i.e., Arginine). The DRY motif may be modified in whole or in part. Modifications of this motif can form a constitutively desensitized receptor.

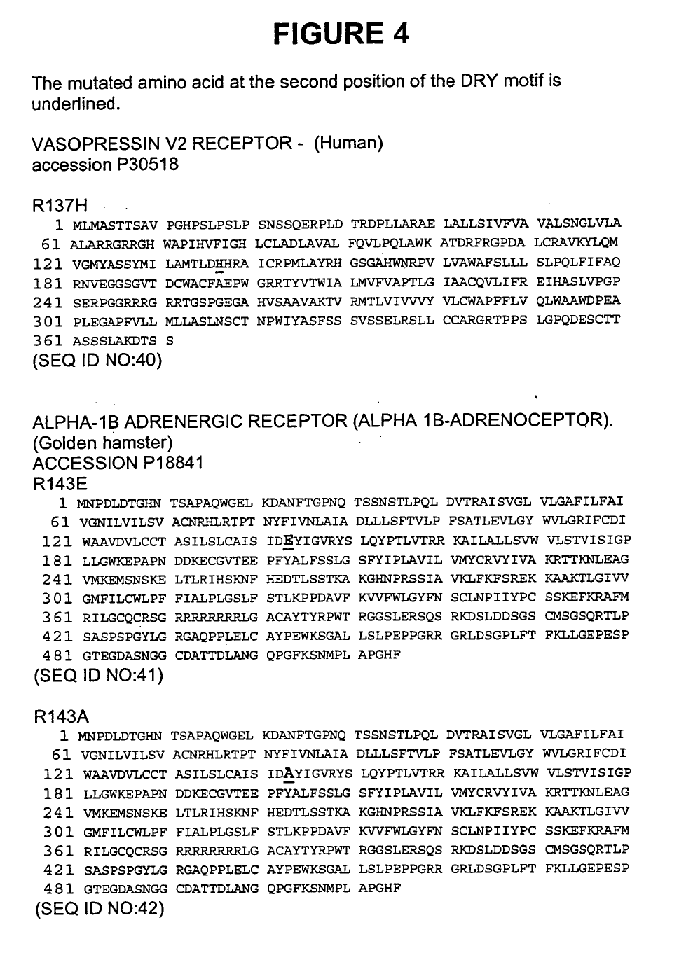

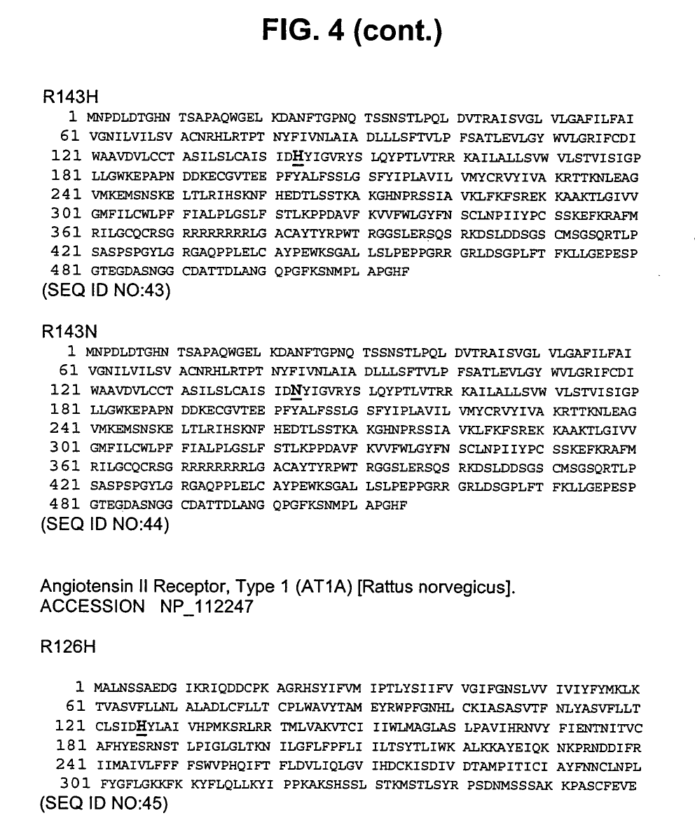

By way of example, the V2R has a DRY motif at amino acids 136-138. Modifications of the DRY motif may promote agonist-independent formation of a GPCR/arrestin complex and constitutive localization to the endocytic vesicles or endosomes. The α_{1B}-AR receptor comprises a DRY motif at amino acids 142-144 that promotes formation of a GPCR/arrestin complex and localizes to endocytic vesicles or endosomes. The AT_{1A}R receptor comprises a DRY motif at amino acids 125-127 that also promotes formation of a GPCR/arrestin complex and localizes to the endocytic vesicles or endosomes. FIG. 4 illustrates the amino acid sequences of the following GPCRs in which the DRY motif has been modified: V2R, α_{1B}-AR, and AT_{1A}R.

The modified GPCRs of the present invention include GPCRs that have been modified in the DRY motif to localize to endocytic vesicles or endosomes in an agonist-independent manner. The polypeptide sequences of the modified GPCRs of the present invention include sequences having one or more additions, deletions, substitutions, or mutations. These mutations may be substitution mutations made in a conservative manner (i.e., by changing the codon from an amino acid belonging to a grouping of amino acids having a particular size or characteristic to an amino acid belonging to the same grouping). Such a conservative change generally leads to less change in the structure and function of the resulting protein. The present invention should be considered to include sequences containing conservative changes that do not significantly alter the activity or binding characteristics of the resulting protein. The present invention should also be considered to include sequences containing non-conservative changes that do not significantly alter the activity or binding characteristics of the resulting protein.

To create a modified GPCR containing a modified DRY motif according to the present invention, a GPCR comprising a DRY motif may have one or more additions, substitutions, deletions, or mutations of amino acid residues in its DRY motif such that the modified GPCR is a constitutively desensitized receptor. By way of example, discrete point mutations of the amino acid residues may be made to provide a modified GPCR. By way of example, three consecutive amino acids may be mutated to provide a modified GPCR. By way of example, the Arginine may be mutated to any amino acid other than Lysine such as, for example, Alanine, Glutamate, Aspartate, Asparagine, or Histidine, to provide a modified GPCR.

In addition, to create a modified GPCR containing a modified DRY motif, mutations may be made in a nucleic acid sequence of a GPCR such that a particular codon is changed to a codon that codes for a different amino acid. Such a mutation is generally made by making the fewest nucleotide changes possible. A substitution mutation of this sort can be made to change an amino acid in the resulting protein to create a modified DRY motif forming a constitutively desensitized receptor. Also by way of example, discrete point mutations of the nucleic acid sequence may be made.

Furthermore, to provide modified GPCRs of the present invention, a GPCR that binds arrestin in an agonist-dependent manner may also have its DRY motif, in whole or in part, exchanged with that of a GPCR having a modified DRY motif that forms a constitutively desensitized receptor. For example, the DRY motif of a GPCR that binds arrestin in an agonist-dependent manner may be exchanged at an amino acid residue in close proximity to the DRY motif.

Modified GPCRs may be generated by molecular biological techniques standard in the genetic engineering art, including but not limited to, polymerase chain reaction (PCR), restriction enzymes, expression vectors, plasmids, and the like. By way of example, vectors may be designed to enhance the agonist-independent affinity of a GPCR for arrestin. PCR amplified DNA fragments of a GPCR to be modified may be digested by appropriate restriction enzymes and subcloned into the vector, such as pcDNA3.1/zeo or pEGFP-N3. Modifications of the DNA may be introduced by standard molecular biological techniques as described above.

As may be shown by standard receptor binding assays, the modified GPCRs are essentially indistinguishable from their wild-type counterparts except for an agonist-independent affinity for arrestin, and thus, constitutive endosomal localization. For example, the modified GPCRs possess similar affinity for antagonists or inverse agonists, and the like.

By way of example, V2R may have a modification R137H (FIG. 3) resulting in modified endocytic targeting. The modified DRY motif of the V2R R137H can be used to replace the DRY motif of other GPCRs. This three amino acid sequence may be located near the cytoplasmic boundary of the third transmembrane region and the second intracellular loop. Modified GPCRs containing a mutation of the R of the DRY motif have an increased affinity for arrestin and colocalize with arrestin in endocytic vesicles or endosomes in an agonist-independent manner.

2. GPCRs with Increased Phosphorylation Sites

GPCRs that do not naturally recruit arrestin to endosomes or do not even naturally recruit arrestin to the plasma membrane may be modified to comprise one or more sites of phosphorylation (e.g., clusters of phosphorylation sites) properly positioned in their carboxyl-terminal tail or properly positioned at other positions in the amino acid sequence (e.g., in the third intracellular loop). This modification allows the modified GPCR to form a stable complex with an arrestin that will internalize into endosomes.

The modified GPCRs of the present invention include GPCRs comprising one or more modifications in their carboxyl-terminal tail. These modifications may comprise inserting one or more sites of phosphorylation (e.g., clusters of phosphorylation sites) within certain regions of the carboxyl-terminal tail, as described in U.S. patent application Ser. No. 09/993,844, filed Nov. 5, 2001, the content of which is hereby incorporated by reference herein in its entirety. As such, the carboxyl-terminal tail may be modified in whole or in part. The carboxyl-terminal tail of many GPCRs begins shortly after a conserved NPXXY motif that marks the end of the seventh transmembrane domain (i.e. what follows the NPXXY motif is the carboxyl-terminal tail of the GPCR). The carboxyl-terminal tail of many GPCRs comprises a putative site of palmitoylation approximately 10 to 25 amino acid residues (e.g., 15 to 20 amino acid residues) downstream of the NPXXY motif. This site is typically one or more cysteine residues. The carboxyl-terminal tail of a GPCR may be relatively long, relatively short, or virtually non-existent. The carboxyl-terminal tail of a GPCR determines the affinity of arrestin binding.

Specific amino acid motifs in the carboxyl-terminal tail promote formation of a stable GPCR/arrestin complex and thus ultimately may promote recruitment of arrestin to endosomes. These amino acid motifs comprise one or more amino acids (e.g., clusters of phosphorylation sites) that may be efficiently phosphorylated and thus readily function as phosphorylation sites. The clusters of amino acids may occupy two out of two, two out of three, three out of three, three out of four positions, four out of four, four out of five positions, five out of five, and the like consecutive amino acid positions. Accordingly, the clusters of amino acids that promote formation of a stable GPCR/arrestin complex are “clusters of phosphorylation sites.” These clusters of phosphorylation sites may be clusters of serine and threonine residues.

GPCRs that form stable complexes with arrestin comprise one or more sites of phosphorylation (e.g., clusters of phosphorylation sites). In addition to the presence of the one or more sites of phosphorylation (e.g., clusters of phosphorylation sites) the sites must be properly positioned within the carboxyl-terminal tail to promote formation of a stable GPCR/arrestin complex. To promote formation of a stable GPCR/arrestin complex, the one or more sites of phosphorylation (e.g., one or more clusters of phosphorylation) may be approximately 15 to 35 (e.g., 15 to 25) amino acid residues downstream of a putative site of palmitoylation of the GPCR. In addition, the one or more sites of phosphorylation (e.g., one or more clusters of phosphorylation, may be approximately 20 to 55 (e.g., 30 to 45) amino acid residues downstream of the NPXXY motif of the GPCR. GPCRs containing one or more sites of phosphorylation (e.g., clusters of phosphorylation sites) properly positioned are typically Class B receptors.

By way of example, it has been discovered that the V2R receptor comprises a cluster of phosphorylation sites (SSS) that promotes formation of a stable GPCR/arrestin complex at 19 amino acid residues downstream of the putative site of palmitoylation and 36 amino acid residues downstream of the NPXXY motif. The NTR-2 receptor comprises a cluster of phosphorylation sites (STS) that promotes formation of a stable GPCR/arrestin complex at 26 amino acid residues downstream of the putative site of palmitoylation and 45 amino acid residues downstream of the NPXXY motif. The oxytocin receptor (OTR) receptor comprises two clusters of phosphorylation sites (SSLST and STLS) that promote formation of a stable GPCR/arrestin complex, one at 20 amino acid residues downstream of the putative site of palmitoylation and 38 amino acid residues downstream of the NPXXY motif, and the other at 29 amino acid residues downstream of the putative site of palmitoylation and 47 amino acid residues downstream of the NPXXY motif. The substance P receptor (SPR, also known as the neurokinin-1 receptor) comprises a cluster of phosphorylation sites (TTIST) that promotes formation of a stable GPCR/arrestin complex at 32 amino acid residues downstream of the putative site of palmitoylation and 50 amino acid residues downstream of the NPXXY motif.

GPCRs that lack one or more sites of phosphorylation (e.g., clusters of phosphorylation sites) properly positioned within the carboxyl terminal tail form GPCR/arrestin complexes that are less stable and dissociate at or near the plasma membrane. These GPCRs are typically Class A receptors, olfactory receptors, taste receptors, and the like. However, stable GPCR/arrestin complexes may be achieved with GPCRs naturally lacking one or more sites of phosphorylation and having a lower affinity for arrestin by modifying the carboxyl-terminal tails of these receptors. The carboxyl-terminal tails may be modified to include one or more sites of phosphorylation (e.g., one or more clusters of phosphorylation sites) properly positioned within the carboxyl terminal tail.

The modified GPCRs of the present invention include GPCRs that have been modified to have one or more sites of phosphorylation (e.g., one or more clusters of phosphorylation) properly positioned in their carboxyl terminal tails. The polypeptide sequences of the modified GPCRs of the present invention also include sequences having one or more additions, deletions, substitutions, or mutations. These mutations may be substitution mutations made in a conservative manner (i.e., by changing the codon from an amino acid belonging to a grouping of amino acids having a particular size or characteristic to an amino acid belonging to the same grouping). Such a conservative change generally leads to less change in the structure and function of the resulting protein. The present invention should be considered to include sequences containing conservative changes that do not significantly alter the activity or binding characteristics of the resulting protein.

The modified GPCRs of the present invention include GPCRs containing a NPXXY motif, a putative site of palmitoylation approximately 10 to 25 amino acid residues (e.g., 15 to 20 amino acids) downstream of the NPXXY motif, and a modified carboxyl-terminal tail. The modified carboxyl-terminal tail has one or more sites of phosphorylation (e.g., one or more clusters of phosphorylation sites) such that the phosphorylation sites are approximately 15 to 35 (e.g., 15 to 25) amino acid residues downstream of the putative site of palmitoylation of the modified GPCR. The modified carboxyl-terminal tail may have one or more sites of phosphorylation (e.g., one or more clusters of phosphorylation sites) such that the phosphorylation sites are approximately 20 to 55 (e.g., 30 to 45) amino acid residues downstream of the NPXXY of the modified GPCR.

To create a modified GPCR containing a modified carboxyl-terminus region according to the present invention, a GPCR lacking phosphorylation sites or clusters of phosphorylation sites or with a lower or unknown affinity for arrestin may have one or more additions, substitutions, deletions, or mutations of amino acid residues in its carboxyl-terminal tail. These additions, substitutions, deletions, or mutations are performed such that the carboxyl-terminal tail is modified to comprise one or more sites of phosphorylation (e.g., clusters of phosphorylation sites). By way of example, discrete point mutations of the amino acid residues may be made to provide a modified GPCR. By way of example, three consecutive amino acids may be mutated to serine residues to provide a modified GPCR. These mutations are made such that the one or more sites of phosphorylation (e.g., clusters of phosphorylation sites) are properly positioned within the carboxyl terminal tail.

In addition, to create a modified GPCR containing a modified carboxyl-terminal tail region, mutations may be made in a nucleic acid sequence of a GPCR lacking sites of phosphorylation or clusters of phosphorylation sites or with a lower or unknown affinity for arrestin such that a particular codon is changed to a codon that codes for a different amino acid (e.g., a serine or threonine). Such a mutation is generally made by making the fewest nucleotide changes possible. A substitution mutation of this sort can be made to change an amino acid in the resulting protein to create one or more sites of phosphorylation (e.g., clusters of phosphorylation sites). Also by way of example, discrete point mutations of the nucleic acid sequence may be made. The phosphorylation sites are positioned such that they are located approximately 15 to 35 amino acid residues downstream of the putative site of palmitoylation of the modified GPCR.

Furthermore, to provide modified GPCRs of the present invention, a GPCR lacking properly positioned phosphorylation sites or with a lower or unknown affinity for arrestin may also have its carboxyl-terminal tail, in whole or in part, exchanged with that of a GPCR having properly positioned clusters of phosphorylation sites. The site of exchange may be after or including the conserved NPXXY motif. As an alternative, a putative site of palmitoylation of a GPCR may be identified at approximately 10 to 25 (e.g., 15 to 20) amino acid residues downstream of the conserved NPXXY motif, and the site of exchange may be after or including the palmitoylated cysteine(s). As discussed below, if a putative site of palmitoylation does not exist, one may be introduced in the GPCR. The carboxyl-terminal tail of a GPCR lacking properly positioned clusters of phosphorylation sites or with a lower or unknown affinity for arrestin may be exchanged at an amino acid residue in close proximity to a putative site of palmitoylation. In one embodiment, the carboxyl-terminal tail of a GPCR lacking properly positioned clusters of phosphorylation sites or with a lower or unknown affinity for arrestin is exchanged at a putative site of palmitoylation approximately 10 to 25 (e.g., 15 to 20) amino acid residues downstream of the NPXXY motif, such that the palmitoylated cysteine residue is maintained. The carboxyl-terminal tail of a GPCR lacking properly positioned clusters of phosphorylation sites may be exchanged in a manner allowing the clusters of phosphorylation sites to be properly positioned within the carboxyl-terminal tail of the modified GPCR. The tails may be exchanged and the modified GPCRs may be constructed accordingly by manipulation of the nucleic acid sequence or the corresponding amino acid sequence.

In a further alternative, the carboxyl-tail of a GPCR, for example a GPCR not containing the NPXXY motif, may be predicted from a hydrophobicity plot and the site of exchange may be selected accordingly. Based on a hydrophobicity plot, one of skill in the art may predict a site where it is expected that the GPCR may anchor in the membrane and then predict where to introduce a putative site of palmitoylation accordingly. Using this technique GPCRs having neither a NPXXY motif nor a putative site of palmitoylation may be modified to create a point of reference (e.g. a putative site of palmitoylation). The introduced putative site of palmitoylation may then be used to position a tail exchange.

The carboxyl-terminal tail used for the exchange may be from a second GPCR having one or more properly positioned clusters of phosphorylation sites and having a putative site of palmitoylation approximately 10 to 25 (e.g., 15 to 20) amino acid residues downstream of a NPXXY motif. The tail as identified may be exchanged, after or including the conserved NPXXY motif. As an alternative, a putative site of palmitoylation of a GPCR may be identified at approximately 10 to 25 (e.g., 15 to 20) amino acid residues downstream of the conserved NPXXY motif, and the tail may be exchanged after or including the palmitoylated cysteine(s). The carboxyl-terminal tail of a GPCR having clusters of phosphorylation sites may be exchanged at an amino acid residue in close proximity to a putative site of palmitoylation. In one embodiment, the carboxyl-terminal tail of a GPCR having clusters of phosphorylation sites is exchanged at a putative site of palmitoylation approximately 10 to 25 (e.g., 15 to 20) amino acid residues downstream of the NPXXY motif, such that the portion of the carboxyl-terminal tail containing the clusters of phosphorylation sites begins at the amino acid residue immediately downstream of the palmitoylated cysteine residue. The carboxyl-terminal tail having clusters of phosphorylation sites used for the exchange may have a marker molecule conjugated to the carboxyl-terminus. The tails may be exchanged and the modified GPCRs may be constructed accordingly by manipulation of the nucleic acid sequence or the corresponding amino acid sequence.

In addition, the carboxyl-terminal tail portion used for the exchange may originate from a polypeptide synthesized to have an amino acid sequence corresponding to an amino acid sequence from a GPCR having one or more sites of phosphorylation (e.g., one or more clusters of phosphorylation sites). The synthesized polypeptide may have a putative site of palmitoylation approximately 10 to 25 (e.g., 15 to 20) amino acid residues downstream of a NPXXY motif. The synthesized polypeptide may have one or more additions, substitutions, mutations, or deletions of amino acid residues that does not affect or alter the overall structure and function of the polypeptide.

Furthermore, the carboxyl-terminal tail portion used for the exchange may originate from a naturally occurring polypeptide recognized to have an amino acid sequence corresponding to an amino acid sequence from a GPCR having one or more clusters of phosphorylation sites. The polypeptide may have a putative site of palmitoylation approximately 10 to 25 (e.g., 15 to 20) amino acid residues downstream of a NPXXY motif. The polypeptide may have one or more additions, substitutions, mutations, or deletions of amino acid residues that does not affect or alter the overall structure and function of the polypeptide.

A modified GPCR containing a modified carboxyl-terminus region may be created by fusing a first carboxyl-terminal tail portion of a GPCR lacking properly positioned clusters of phosphorylation sites or with a lower or unknown affinity for arrestin with a second carboxyl-terminal tail portion of a GPCR or polypeptide having one or more clusters of phosphorylation sites. The second GPCR or polypeptide used for the exchange may have a putative site of palmitoylation approximately 10 to 25 (e.g., 15 to 20) amino acid residues downstream of a NPXXY motif. Accordingly, the modified carboxyl-terminus region of the modified GPCR comprises a portion of a carboxyl-terminal tail from a GPCR lacking properly positioned clusters of phosphorylation sites or with a lower or unknown affinity for arrestin fused to a portion of a carboxyl-terminal tail of a GPCR or polypeptide having clusters of phosphorylation sites. The tail of a GPCR lacking properly positioned clusters of phosphorylation sites may be exchanged after or including the conserved NPXXY motif, and fused to a carboxyl-terminal tail containing clusters of phosphorylation sites, after or including the conserved NPXXY motif. As an alternative, the tail of a GPCR lacking properly positioned clusters of phosphorylation sites may be exchanged after or including the palmitoylated cysteine(s), and fused to a tail containing clusters of phosphorylation sites, after or including the palmitoylated cysteine(s). The tails may be exchanged and the modified GPCRs may be constructed accordingly by manipulation of the nucleic acid sequence or the corresponding amino acid sequence.

In a further alternative, the carboxyl-tail of a GPCR, for example a GPCR not containing the NPXXY motif, may be predicted from a hydrophobicity plot and exchanged accordingly. The site of exchange may be selected according to the hydrophobicity plot. Based on a hydrophobicity plot, one of skill in the art may predict a site where it is expected that the GPCR may anchor in the membrane and then predict where to introduce a putative site of palmitoylation accordingly. Using this technique GPCRs having neither a NPXXY motif nor a putative site of palmitoylation may be modified to create a point of reference (e.g. a putative site of palmitoylation). The introduced putative site of palmitoylation may be then used to position a tail exchange. After introduction of a putative site of palmitoylation, the resulting tail may be fused with a second carboxyl-terminal tail portion of a GPCR or polypeptide having one or more clusters of phosphorylation sites and having a putative site of palmitoylation approximately 10 to 25 (e.g., 15 to 20) amino acid residues downstream of a NPXXY motif.

The modified carboxyl-terminus region of the modified GPCR may be fused at amino acid residues in close proximity to a putative site of palmitoylation. In one embodiment, the modified carboxyl-terminus region of the modified GPCR is fused such that the portion from the first GPCR with a lower affinity for arrestin comprises amino acid residues from the NPXXY motif through a putative site of palmitoylation approximately 10 to 25 (e.g., 15 to 20) amino acid residues downstream of the NPXXY motif and the portion from the second GPCR having clusters of phosphorylation sites and a putative site of palmitoylation approximately 10 to 25 (e.g., 15 to 20) amino acid residues downstream of a NPXXY motif comprises amino acid residues beginning with an amino acid residue immediately downstream of the putative site of palmitoylation of the second GPCR extending to the end of the carboxyl-terminus. Such a fusion allows the clusters of phosphorylation sites to be properly positioned within the carboxyl-terminal tail and allows the modified GPCR to maintain its structure and ability to function.

By way of example, a Class A receptor or an orphan receptor may have a portion of its carboxyl-terminal tail exchanged with a portion of a carboxyl-terminal tail from a known Class B receptor. Further, receptors having virtually non-existent carboxyl-terminal tails, for example, olfactory receptors and taste receptors, may have a portion of their carboxyl-terminal tails exchanged with a portion of a carboxyl-terminal tail from a known Class B receptor. The Class B receptor tail used for these exchanges may have a marker molecule fused to the carboxyl-terminus.

Modified GPCRs may be generated by molecular biological techniques standard in the genetic engineering art, including but not limited to, polymerase chain reaction (PCR), restriction enzymes, expression vectors, plasmids, and the like. By way of example, vectors, such as a pEArrB (enhanced arrestin binding, described in U.S. patent application Ser. No. 09/993,844), may be designed to enhance the affinity of a GPCR lacking clusters of phosphorylation sites for arrestin. To form a vector, such as a pEArrB vector, PCR amplified DNA fragments of a GPCR carboxyl-terminus, which forms stable complexes with arrestin, may be digested by appropriate restriction enzymes and cloned into a plasmid. The DNA of a GPCR, which is to be modified, may also be PCR amplified, digested by restriction enzymes at an appropriate location, and subcloned into the vector, such as pEArrB. When expressed, the modified GPCR will contain a polypeptide fused to the carboxyl-terminus. The polypeptide will comprise clusters of phosphorylation sites. In one embodiment, the polypeptide originates from the GPCR carboxyl-terminus of a receptor that forms stable complexes with arrestin.

Such modified GPCRs may also occur naturally as the result of aberrant gene splicing or single nucleotide polymorphisms. Such naturally occurring modified GPCRs would be predicted to have modified endocytic targeting.

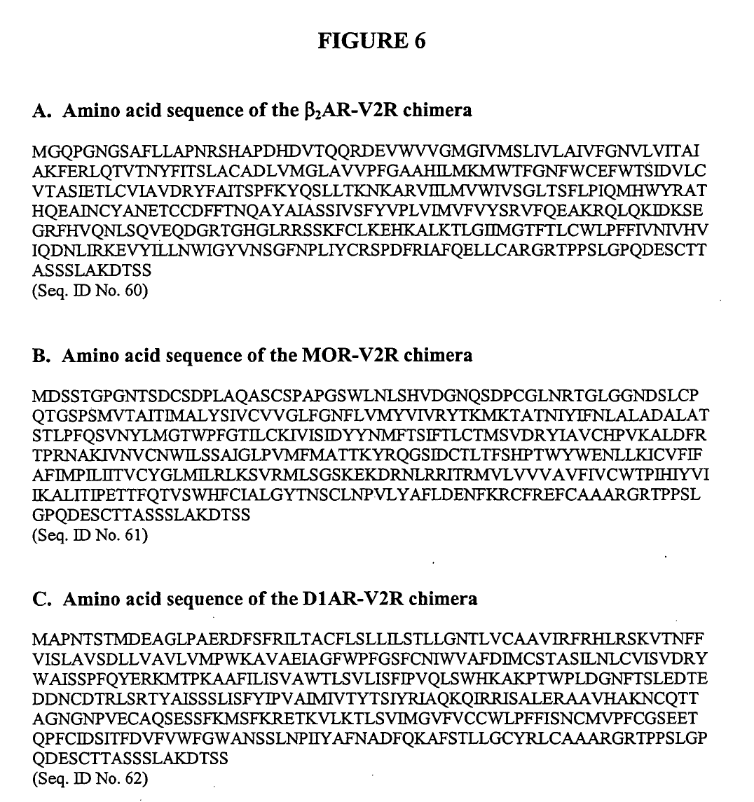

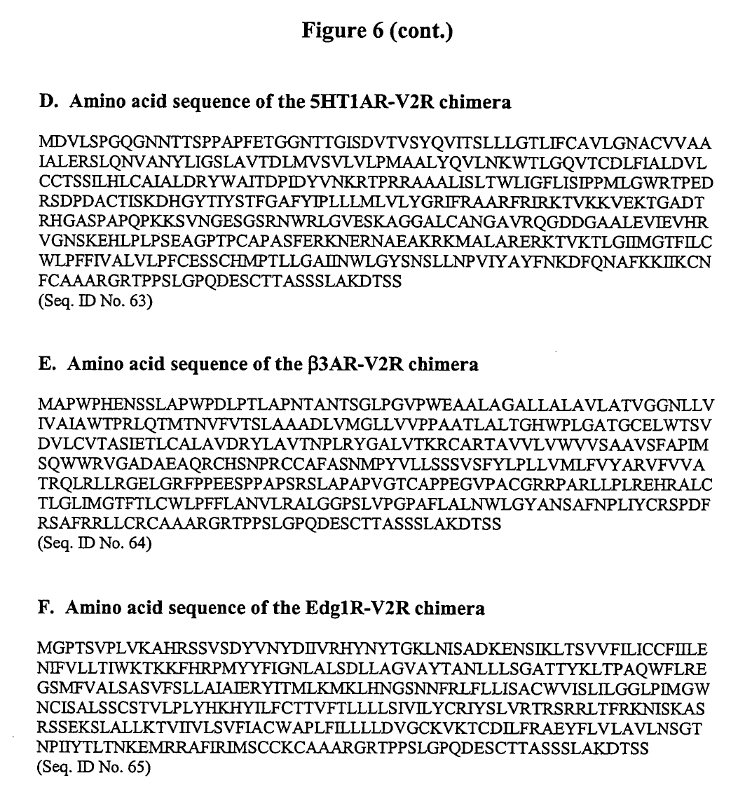

As shown in FIG. 6A, a portion of a β₂AR, a Class A receptor, may be fused to a portion of a V2R receptor (a Class B receptor). As shown in the figure, the first 341 amino acids of the β₂AR, Met-1 through Cys-341 (a putative site of palmitoylation) may be fused to the last 29 amino acids of the V2R carboxyl-terminus (Ala-343 through Ser-371; Ala-343 is immediately following a palmitoylated cysteine). This fusion properly positions the V2R cluster of phosphorylation sites (SSS) within the modified GPCR's tail.

As shown in FIG. 6B, a portion of a mu opioid receptor (MOR), a Class A receptor, may be fused to a portion of a V2R receptor (a Class B receptor). As shown in the figure, the first 351 amino acids of the MOR, Met-I through Cys-351 (a palmitoylated cysteine residue), may be fused to the last 29 amino acid of the V2R carboxyl-terminus (Ala-343 through Ser-371; Ala-343 is immediately following a palmitoylated cysteine). This fusion properly positions the V2R cluster of phosphorylation sites (SSS) within the modified GPCR's tail.

Also as shown in FIG. 6C, a portion of a dopamine D1A receptor (D1AR), a Class A receptor, may be fused to a portion of a V2R receptor. As shown in the figure, the first 351 amino acids of the D1AR, Met-1 through Cys-351 (a palmitoylated cysteine) may be fused to the last 29 amino acid of the V2R carboxyl-terminus (Ala-343 through Ser-371; Ala-343 is immediately following a palmitoylated cysteine). This fusion properly positions the V2R cluster of phosphorylation sites (SSS) within the modified GPCR's tail.

Further as shown in FIG. 6D, a portion of a 5-hydroxytryptamine 1A receptor (5HT1 AR), a Class A receptor, may be fused to a portion of a V2R receptor (a Class B receptor). As shown in the figure, the first 420 amino acids of the 5HT1AR, Met-1 through Cys-420 (a palmitoylated cysteine) may be fused to the last 29 amino acid of the V2R carboxyl-terminus (Ala-343 through Ser-371; Ala-343 is immediately following a palmitoylated cysteine). This fusion properly positions the V2R cluster of phosphorylation sites (SSS) within the modified GPCR's tail.

As shown in FIG. 6E, a portion of a β3-adrenergic receptor (β3AR), a Class A receptor, may be fused to a portion of a V2R receptor (a Class B receptor). As shown in the figure, the first 363 amino acids of the β3AR, Met-1 through Cys-363 (a palmitoylated cysteine) may be fused to the last 29 amino acid of the V2R carboxyl-terminus (Ala-343 through Ser-371; Ala-343 is immediately following a palmitoylated cysteine). This fusion properly positions the V2R cluster of phosphorylation sites (SSS) within the modified GPCR's tail.

Finally as shown in FIG. 6F, a portion of a endothelial differentiation, sphingolipid GPCR 1 (Edg1R), a Class A receptor, may be fused to a portion of a V2R receptor (a Class B receptor). As shown in the figure, the first 331 amino acids of the Edg1R, Met-1 through Cys-331 (a palmitoylated cysteine) may be fused to the last 29 amino acid of the V2R carboxyl-terminus (Ala-343 through Ser-371; Ala-343 is immediately following a palmitoylated cysteine). This fusion properly positions the V2R cluster of phosphorylation sites (SSS) within the modified GPCR's tail.

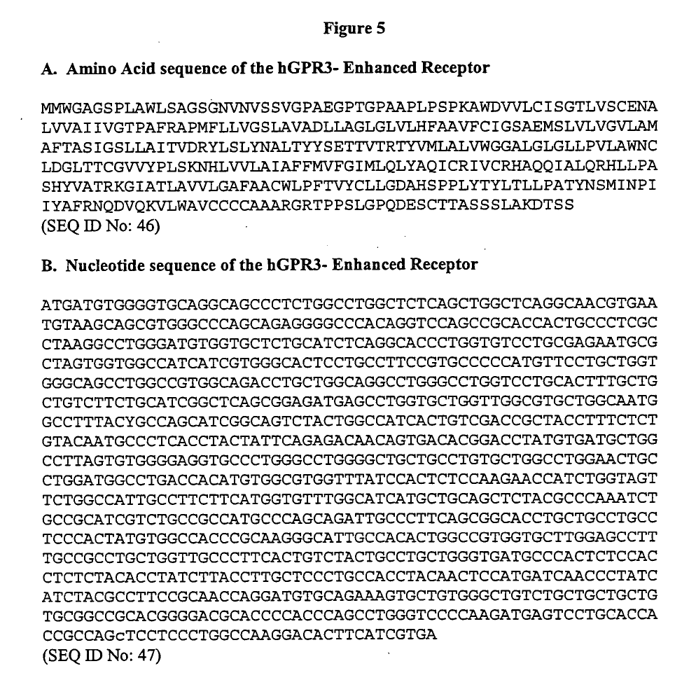

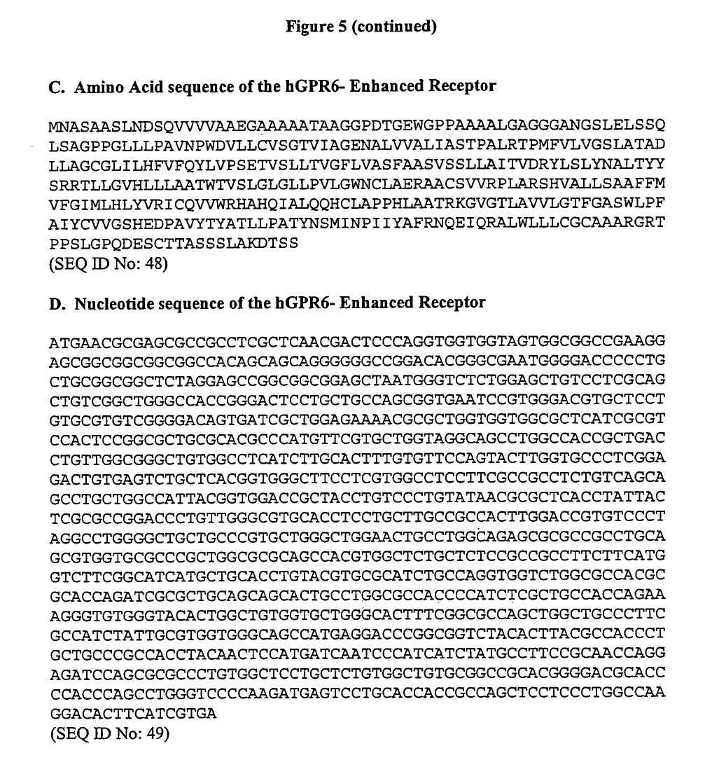

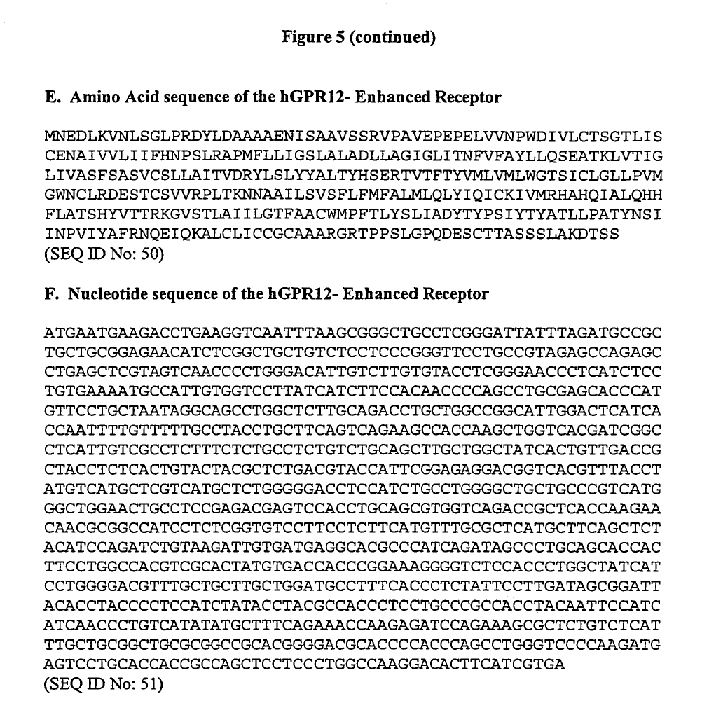

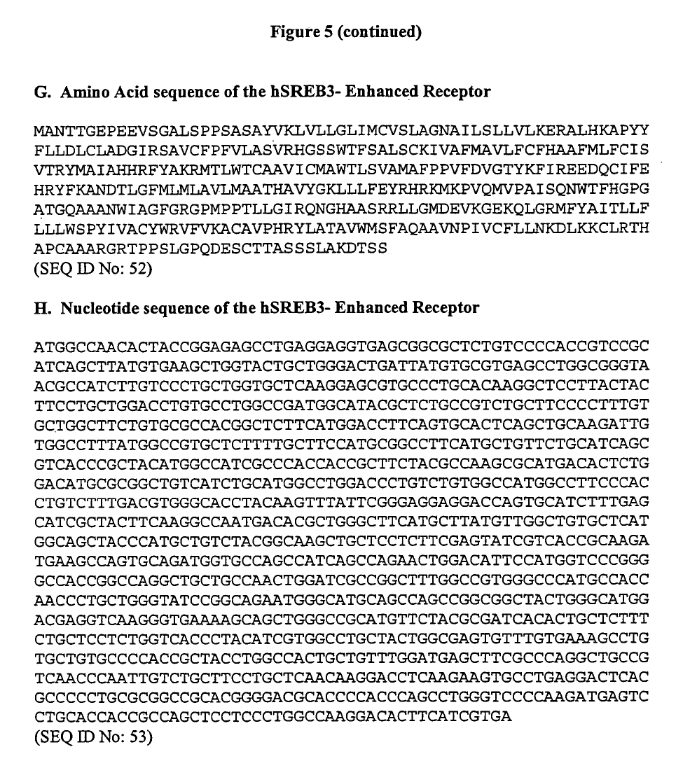

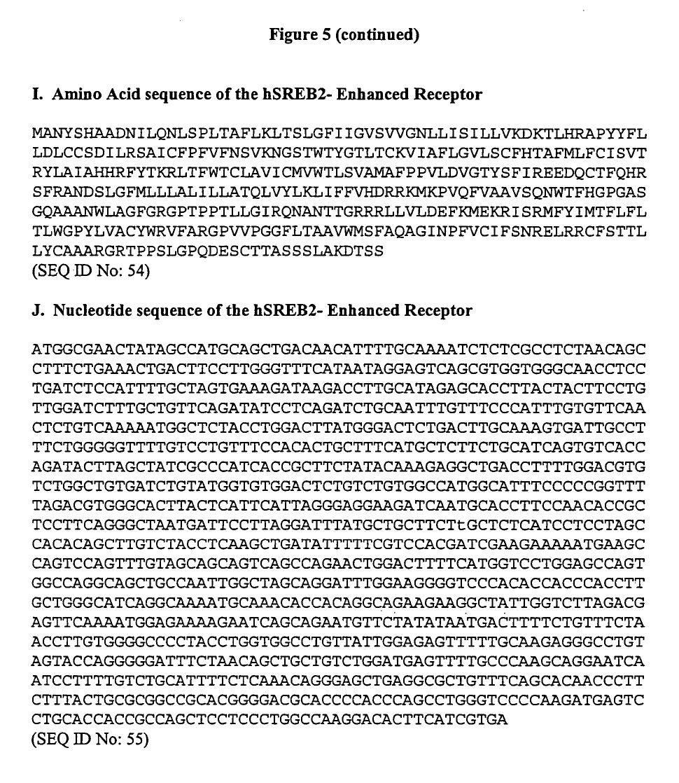

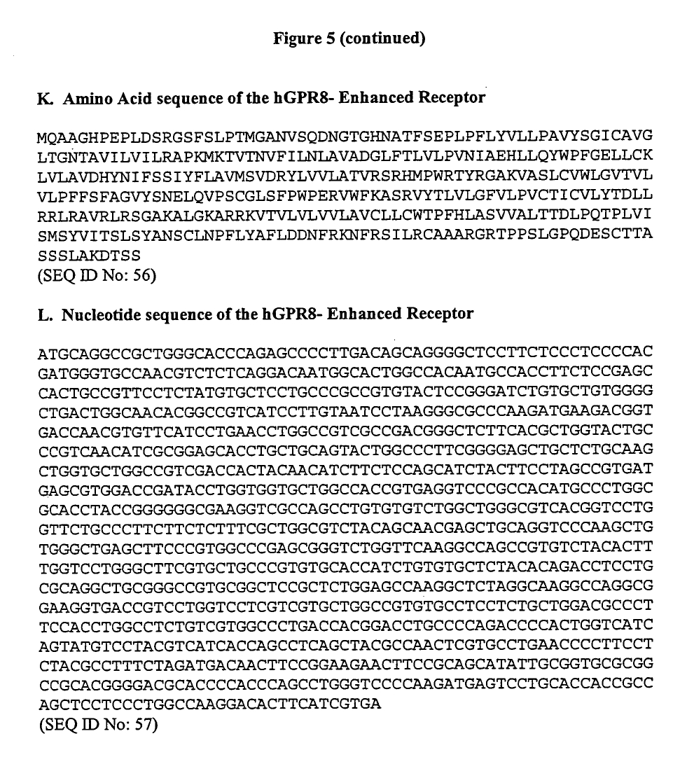

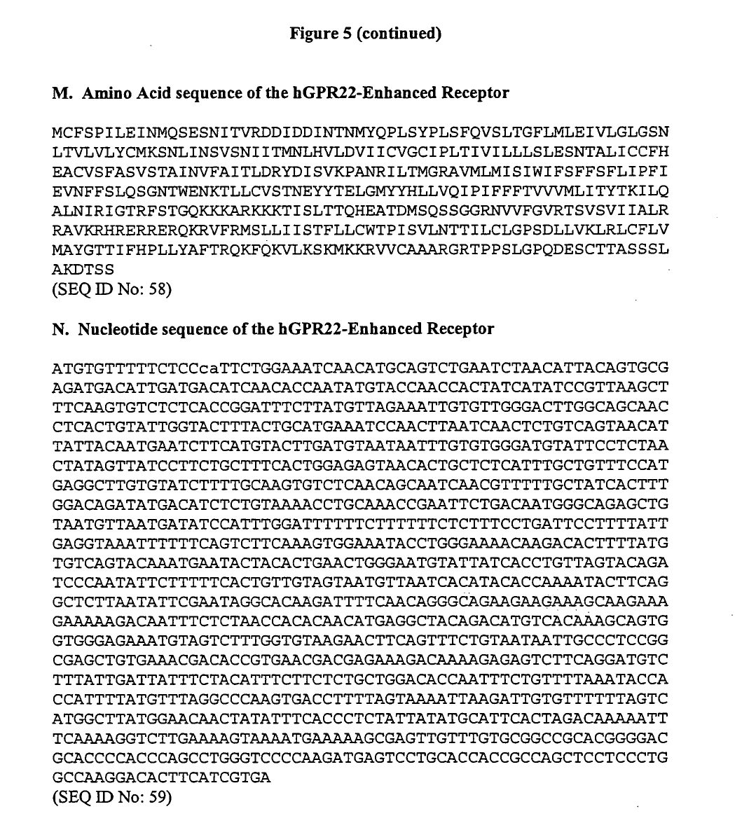

The modified GPCRs described in U.S. Provisional Patent Application No. 60/401,698, filed Aug. 7, 2002, the content of which is hereby incorporated by reference herein in its entirety, may also be used in the present invention. The GPCRs described in U.S. Provisional Patent Application No. 60/401,698 include the following receptors that have enhanced affinity for arrestin: hGPR3E, hGPR6E, hGPR12E, hGPR8E, hGPR22E, hSREB2E, and hSREB3E. The “E” stands for “enhanced arrestin binding”. Each of these modified GPCRs contains a properly positioned cluster of phosphorylation sites (SSS) within the modified GPCR's tail. FIG. 5 lists the amino acid and nucleic acid sequences for these GPCRs.

As may be shown by standard receptor binding assays, the modified receptors are essentially indistinguishable from their wild-type counterparts except for an increased affinity for arrestin and thus an increased stability of their complex with arrestin and in their ability to traffic with arrestin and in their ability to recycle and resensitize. For example, the modified receptors are appropriately expressed at the membrane and possess similar affinity for agonists or ligands.

3. Other Modified GPCRs

Other modified GPCRs may also be used in the present invention so long as the modified GPCRs are capable of participating in the GPCR desensitization pathway such that GPCR desensitization inhibitory activity of a test composition may be determined. For example, the human β₂AR-E-Y326A containing a point mutation (i.e., the Tyrosine residue 326 converted to Alanine) may be used in the present invention. β2AR-E-Y326A is described in U.S. Provisional Patent Application No. 60/401,698. The “E” indicates that the GPCR has been modified as described above to have enhanced affinity for arrestin. The Y326A mutation causes the GPCR to be dependent on overexpressed GRK for phosphorylation and subsequent desensitization.

Cells

Cells useful in the present invention include eukaryotic and prokaryotic cells, including, but not limited to, bacterial cells, yeast cells, fungal cells, insect cells, nematode cells, plant cells, and animal cells. Suitable animal cells include, but are not limited to, HEK cells, HeLa cells, COS cells, U20S cells, CHO-K1 cells, and various primary mammalian cells. An animal model expressing one or more conjugates described above (e.g., a conjugate of an arrestin and a marker molecule) throughout its tissues or within a particular organ or tissue type, may be useful in the methods of the present invention.

Cells useful in the present invention include those that express a known GPCR, a variety of known GPCRs, an unknown GPCR, a variety of unknown GPCRs, a modified GPCR, a variety of modified GPCRs, and combinations thereof. A cell that expresses a GPCR is one that contains the GPCR as a functional receptor in its cell membrane. The cells may naturally express the GPCRs, may be genetically engineered to express the GPCRs at varying levels of expression, or may be genetically engineered to inducibly express the GPCRs. As one skilled in the art readily would understand, the cells may be genetically engineered to express GPCRs by molecular biological techniques standard in the genetic engineering art.

Conjugates

In the methods of the present invention, each of the cells comprises one or more conjugates of a marker molecule and a protein associated with the GPCR desensitization pathway of one or more GPCRs that are being used in the cell to screen a test composition for GPCR desensitization inhibitory activity. For example, one or more of the cells may comprise a conjugate of an arrestin protein and a marker molecule and/or a conjugate of a GPCR and a marker molecule.

All forms of arrestin, both naturally occurring and engineered variants, including but not limited to, visual arrestin, β-arrestin 1 and β-arrestin 2, may be used in the present invention.

Marker molecules that may be used to conjugate with the arrestin include, but are not limited to, molecules that are detectable by spectroscopic, photochemical, radioactivity, biochemical, immunochemical, calorimetric, electrical, and optical means, including but not limited to, bioluminescence, phosphorescence, and fluorescence. These marker molecules should be biologically compatible molecules and should not compromise the ability of the arrestin to interact with the GPCR system, and the interaction of the arrestin with the GPCR system must not compromise the ability of the marker molecule to be detected. Marker molecules include radioisotopes, epitope tags, affinity labels, enzymes, fluorescent groups, chemiluminescent groups, and the like. Marker molecules include molecules that are directly or indirectly detected as a function of their interaction with other molecule(s) as well as molecules detected as a function of their location or translocation. In some embodiments, the marker molecules are optically detectable marker molecules, including optically detectable proteins, such that they may be excited chemically, mechanically, electrically, or radioactively to emit fluorescence, phosphorescence, or bioluminescence. Optically detectable marker molecules include, for example, beta-galactosidase, firefly luciferase, bacterial luciferase, fluorescein, Texas Red, horseradish peroxidase, alkaline phosphatase, and rhodamine-conjugated antibody. In other embodiments, the optically detectable marker molecules are inherently fluorescent molecules, such as fluorescent proteins, including, for example, Green Fluorescent Protein (GFP).

The marker molecule may be conjugated to the arrestin protein by methods as described in U.S. Pat. Nos. 5,891,646 and 6,110,693. The marker molecule may be conjugated to the arrestin at the front-end, at the back-end, or in the middle. In some embodiments, the marker molecules are molecules that are capable of being synthesized in the cell. The cell can be transfected with DNA so that the conjugate of arrestin and a marker molecule is produced within the cell. As one skilled in the art readily would understand, cells may be genetically engineered to express the conjugate of arrestin and a marker molecule by molecular biological techniques standard in the genetic engineering art.

The GPCRs used in the present invention may also be conjugated with a marker molecule. In some embodiments, the carboxyl-terminus of the GPCR is conjugated with a marker molecule. A carboxyl-terminal tail conjugated or attached to a marker molecule can be used in a carboxyl-terminal tail exchange to provide a modified GPCR.

If the GPCR is conjugated with a marker molecule, proximity of the GPCR with the arrestin may be readily detected. In addition, if the GPCR is conjugated with a marker molecule, compartmentalization of the GPCR with the arrestin may be readily confirmed. The marker molecule used to conjugate with the GPCRs may include those as described above, including, for example, optically detectable marker molecules, such that they may be excited chemically, mechanically, electrically, or radioactively to emit fluorescence, phosphorescence, or bioluminescence. Optically detectable marker molecules may be detected by, for example, immunofluorescence, luminescence, fluorescence, and phosphorescence.

For example, the GPCRs may be antibody labeled with an antibody conjugated to an immunofluorescence molecule or the GPCRs may be conjugated with a luminescent donor. In particular, the GPCRs may be conjugated with, for example, luciferase, for example, Renilla luciferase, or a rhodamine-conjugated antibody, for example, rhodamine-conjugated anti-HA mouse monoclonal antibody. The carboxyl-terminal tail of the GPCR may be conjugated with a luminescent donor, for example, luciferase. The GPCR also may be conjugated with GFP (e.g., at the carboxyl-terminal tail of the GPCR) as described in L. S. Barak et al. “Internal Trafficking and Surface Mobility of a Functionally Intact β₂-Adrenergic Receptor-Green Fluorescent Protein Conjugate”, Mol. Pharm. (1997) 51, 177-184.

Modifications to Other Proteins

Other proteins may also be modified for use in the screening methods described herein. For example, an arrestin may have one or more modifications (e.g., genetic mutations or other functional alterations) in any part thereof that either enhances or reduces the affinity of the arrestin for the GPCR. In addition, AP-2 protein and clathrin may have one or more modifications in any part thereof that either enhances or reduces the ability of arrestins bound to a receptor to remain bound. The altered affinity of arrestin for the GPCR may lead to a constitutively desensitized GPCR. Additionally, the expression of arrestin may be increased with respect to wild-type, which may lead to a constitutively desensitized GPCR.

Further, a G protein-coupled receptor kinase (GRK) may have one or more modifications in any part thereof that either enhances phosphorylation of a GPCR (leading to enhanced affinity of the GPCR for arrestin) or reduces phosphorylation of a GPCR. The modified GRK may lead to constitutive desensitization. One such modified GRK includes GRK-C20, described in U.S. Provisional Patent Application No. 60/401,698. Additionally, the expression of GRKs may be increased with respect to wild-type, which may lead to a constitutively desensitized GPCR.

In addition, a protein phosphatase may have one or more modifications in any part thereof that either enhances or reduces dephosphorylation of a GPCR, leading to enhanced or reduced affinity of the GPCR for arrestin. Modification(s) in a protein phosphatase may lead to constitutive desensitization. Protein phosphatases that may be involved in the GPCR signaling pathway, include, for example, calcium regulated serine threonine phosphatases. Examples of Ca-regulated serine threonine phosphatases include the PPEF1/PPEF2 family of phosphatases.

Methods of Detection

Methods of detecting the intracellular location, concentration, or translocation of a conjugate of a protein associated with the GPCR desensitization pathway (e.g., an arrestin protein or a GPCR) and a marker molecule or interaction of the conjugate with another molecule (e.g., interaction of an arrestin protein with a GPCR) will vary depending upon the marker molecule(s) used. For example, the methods of detecting the intracellular location, concentration, or translocation of the conjugate of an arrestin protein and a marker molecule or of a conjugate of a GPCR and a marker molecule, including for example, the concentration of arrestin at a cell membrane, colocalization of arrestin with GPCR in endocytic vesicles or endosomes, and concentration of arrestin in clathrin-coated pits, and the like, will vary depending on the marker molecule(s) used. One skilled in the art readily will be able to devise detection methods suitable for the marker molecule(s) used. For optically marker molecules, any optical method may be used where a change in the fluorescence, bioluminescence, or phosphorescence may be measured due to a redistribution or reorientation of emitted light. Such methods include, for example, polarization microscopy, bioluminescence resonance energy transfer (BRET), fluorescence resonance energy transfer (FRET), evanescent wave excitation microscopy, and standard or confocal microscopy.

In one embodiment, an arrestin protein may be conjugated to a GFP and the arrestin-GFP conjugate may be detected by confocal microscopy. In another embodiment, an arrestin protein may be conjugated to a GFP and a GPCR may be conjugated to an immunofluorescent molecule; the conjugates may be detected by confocal microscopy. In an additional embodiment, an arrestin protein may be conjugated to a GFP, and the carboxy-terminus of a GPCR may be conjugated to a luciferase. These conjugates can be detected by BRET. In a further embodiment, an arrestin protein may be conjugated to a luciferase, and a GPCR may be conjugated to a GFP. The luciferase/GFP conjugates may be detected by BRET.

Methods of detection that may be used with the methods of the present invention are also described in U.S. patent application Ser. No. 10/095,620, U.S. Pat. No. 5,891,646 and U.S. Pat. No. 6,110,693, the contents of which are hereby incorporated by reference herein in their entirety.

Secondary Assays

After receiving an indication of non-receptor-specific GPCR desensitization inhibitory activity for a particular test composition using one or more of the methods for screening described above, various secondary assays may be performed to further characterize the activity of the test composition and/or to confirm (i.e., give a further indication) that the GPCR desensitization inhibitory activity of the test composition is not receptor specific. Assays that may be used to further characterize the activity of the test composition include, but are not limited to in vivo and/or in vitro kinase activity assays, including GRK activity assays. Assays that may be used to confirm that the GPCR desensitization inhibitory activity of the test composition is not receptor specific include, but are not limited to, GPCR ligand binding assays. In addition, any other assays known to those skilled in the art may also be used to characterize the activity, confirm the non-receptor specificity, or characterize other properties of a test composition that has been indicated to have non-receptor-specific GPCR desensitization inhibitory activity. Kinase assays and GPCR ligand binding assays are described more fully below.

1. Kinase Assays

Kinase assays (e.g., whole cell phosphorylation or in vitro phosphorylation assays) may be used to further characterize a test composition that has given an indication of non-receptor-specific GPCR desensitization inhibitory activity to determine if the test composition is a kinase inhibitor. Such a determination may be made by assaying for the ability of the test composition to inhibit phosphorylation by a specific kinase (e.g., a specific GRK). In addition, multiple kinase assays could be performed to further characterize the test composition to determine which, if any, kinases the composition inhibits. In one particular embodiment, a GRK assay is performed using a specific GRK and a GPCR for which the GRK is specific (as the substrate for phosphorylation) to determine whether the composition is an inhibitor of that specific GRK. Kinase phosphorylation assays are known in the art. For example, U.S. Pat. No. 6,096,705 describes an assay for βAR kinase activity.

2. GPCR Ligand Binding Assays

GPCR ligand binding assays may be used to confirm (i.e., give a further indication) that the GPCR desensitization inhibitory activity of a test composition is not receptor specific. A known ligand of a specific GPCR is labeled, and then the labeled ligand and a test composition are assayed with the specific GPCR. Any suitable label may be used such as for example, radioactive labels such as ³H, ³²P, etc., and nonradioactive labels. The label may comprise any marker molecule as defined above. The labeled ligand (e.g., radiolabeled ligand) and the test composition are allowed to compete for binding with the specific GPCR. The assay may be conducted using cells comprising the specific GPCR or may be conducted using a cell-free format (e.g., using membrane preparations containing the specific GPCR). After the test composition and the labeled ligand are allowed to compete for binding with the specific GPCR, the amount of labeled ligand bound to the GPCR is measured. Based on the amount of ligand bound to the GPCR, a determination may be made as to whether the test composition competitively binds to the GPCR. The measurement may be compared to a control assay where only the labeled ligand is allowed to bind with the GPCR.

The specific GPCR used in the ligand binding assay may be one of the GPCRs used in the method of screening the composition. In addition, multiple GPCR ligand binding assays may be conducted in order to test each GPCR used in the method of screening the composition. For example, where three cells are used in a method of screening a composition for non-receptor-specific GPCR desensitization inhibitory activity (e.g., a first cell having a first GPCR, a second cell having a second GPCR different from the first GPCR, and a third cell having a third GPCR different from the first and second GPCRs), a separate GPCR ligand binding assay may be conducted for each GPCR to verify that the test composition is not specific for any of the receptors.

### EXAMPLE

The invention will be further explained by the following illustrative example that is intended to be non-limiting.

Introduction

Fifteen compounds were evaluated for their ability to inhibit desensitization in a non-receptor-specific manner (i.e., the fifteen compounds were screened for non-receptor-specific GPCR desensitization inhibitory activity). Each of the fifteen compounds was separately screened for GPCR desensitization inhibitory activity against HA-tagged human β₂-adrenergic receptor (β₂AR) and against human vasopressin V₂ receptor (V2R). In addition, the effects of temperature and carbon dioxide (CO₂) were evaluated with respect to each compound.

Cells

The assay was carried out using two different “double stable” human osteosarcoma cell (U2OS) lines. One cell line (U2OS-β₂AR/ArrGFP) stably expressed β₂AR and an arrestin-GFP conjugate of the Renilla reniformis green fluorescent protein fused in frame to the carboxyl terminus of rat β-arrestin2. The other cell line (U2OS-V2R/ArrGFP) stably expressed V2R and the same arrestin-GFP conjugate.

The double stable cell lines were generated using plasmid DNA constructs as described in Oakley et al., “The Cellular Distribution of Fluorescently Labeled Arrestins Provides a Robust, Sensitive, and Universal Assay for Screening G Protein-Coupled Receptors”, Assay and Drug Development Technologies, Volume 1, Number 1-1, pp. 21-30, 2002.

Test Compounds

The fifteen test compounds used for screening in the example are listed below in Table 1. The table lists the compound name, the vendor of the compound, the vendor product number of the compound, the identifier used herein to refer to the compound, and the Chemical Abstract Service number (CAS#) of the compounds.

TABLE 1
Test compounds

| | Vendor | |
|---|---|---|
| Compound name | (vendor product #) | CAS # |
| Ketotifen fumarate salt | SIGMA-RBI (K2628) | 34580-13-7 |
| 3-tert-Butyl-2-propionyl-2H- | Menai (E343) | 437710-47-9 |
| indeno[1,2-c]pyrazol-4-one | | |
| 5-[2-(5-Nitro-furan-2-yl)-vinyl]- | Maybridge (NRB 00507) | 18873-34-2 |
| furan-2-carboxylic acid methyl | | |
| ester | | |
| 4-amino-2-methyl-N-[2-[(2- | not commercially | 403514-61-4 |
| nitrophenyl)thio]phenyl]-5- | available | |
| Pyrimidinemethanamine | | |
| RO-31-7549 | Calbiochem (557508) | 125313-65-7 |
| RO-31-8425 | Calbiochem (557514) | 131848-97-0 |
| Bisindolylmaleimide III | Alexis (270-051-M005) | 137592-43-9 |
| Bisindolylmaleimide VI | Alexis (270-054-M005) | 137592-46-2 |
| Bisindolylmaleimide VII | Alexis (270-055-M005) | 137592-47-3 |
| Bisindolylmaleimide II | Calbiochem (203292) | 137592-45-1 |
| Bisindolylmaleimide III, | Calbiochem (203294) | |
| Hydrochloride | | |
| Bisindolylmaleimide IV | Calbiochem (203297) | 119139-23-0 |
| 6-Fluoronorepinephrine, | Sigma-Aldrich (B-012) | 70952-50-0 |
| Hydrochloride | | |
| Sanguinarine Chloride (S5890) | Sigma-Aldrich (S5890) | 2447-54-3 |
| Sanguinarine Chloride (LOPAC) | Sigma-Aldrich (obtained | 2447-54-3 |
| | from Sigma as part of | |
| | the LOPAC Library | |
| | of Pharmacologically | |
| | Active Compounds as a | |
| | solution in DMSO) | |

Methods

The two cell lines (U2OS-β₂AR/ArrGFP and U2OS-V2R/ArrGFP) were separately plated at 7000 cells/well on 384 well glass bottom plates. Two plates of U2OS-β₂AR/ArrGFP cells were prepared and two plates of U2OS-V2R/ArrGFP cells were prepared. The cells were then incubated in a humidified environment at 37° C. in 5% CO₂/95% O₂ for 24 hours prior to screening.

Growth media was replaced with serum free media (i.e., Eagle's minimum essential medium (EMEM) buffered with 10 mM HEPES (N-2-Hydroxyethylpiperazine-N′-2-ethanesulfonic acid)) 24 hours after plating of the cells. One plate for each cell line was incubated for 10 minutes at room temperature and atmospheric CO₂ (referred to as the “β₂AR RT” and “V2R RT” plates) and the other plate for each cell line was incubated for 10 minutes at 37° C. and 5% CO₂/95% O₂ (referred to as the “β₂AR CO2” and “V2R CO2” plates).

Each test compound was solvated using 100% dimethyl sulfoxide (DMSO). Multiple solutions of each compound were prepared at varying concentrations for testing in separate wells of each plate. If needed, the solutions were sonicated and heated to increase solubility. After preparation of the compound solutions, they were shielded from light.

Each of the solutions of varying concentrations of the fifteen compounds was added to a well on each of the four plates. The β₂AR RT and V2R RT plates were incubated for 30 minutes at room temperature and atmospheric CO₂. The β₂AR CO2 and V2R CO2 plates were incubated for 30 minutes at 37° C. and 5% CO₂/95% O₂.

Agonist was then added to each well. 100 nM isoproterenol (0.4% weight/volume ascorbic acid) was used for the β₂AR plates and 100 nM arginine vasopressin was used for the V2R plates. The β₂AR RT and V2R RT plates were incubated for 30 minutes at room temperature and atmospheric CO₂. The β₂AR CO2 and V2R CO2 plates were incubated for 30 minutes at 37° C. and 5% CO₂/95% O₂.

Each assay was then terminated using 1% paraformaldehyde containing 1 μM DRAQ5 DNA probe to fix the cells. The cells were analyzed using a pre-production model (alpha unit) of the IN Cell Analyzer 3000 (Amersham Biosciences), which is a line scanning, confocal imaging system. The IN Cell Analyzer 3000 was used to quantitate the localization of the arrestin-GFP conjugate for the cells in each well using the Amersham Biosciences granularity analysis GRNO algorithm. This algorithm finds the nucleus of cells and then dilates out a specified distance in which fluorescent spots or “grains” of arrestin-GFP localization are identified based on size and fluorescent intensity. The average of the fluorescent intensity of the identified grains per cell in an acquired image (i.e., Fgrains) was determined for each well on the plates.

Controls

Control wells were used on each plate to determine the basal level of Fgrains for the cells on the different plates (Basal) as well as to determine the maximally stimulated level of Fgrains for the cells on the different plates (Stimulated). The cells in the Basal control wells were subjected to the method described above, but no test compound or agonist was added to the wells. The cells in the Stimulated control wells were subjected to the method described above, including the addition of agonist, but no test compound was added to the wells.

As a positive control, concentration-response curves of propanolol (an antagonist of β₂AR) were run in parallel as well as a block of maximal concentration (1100 nM).

Results

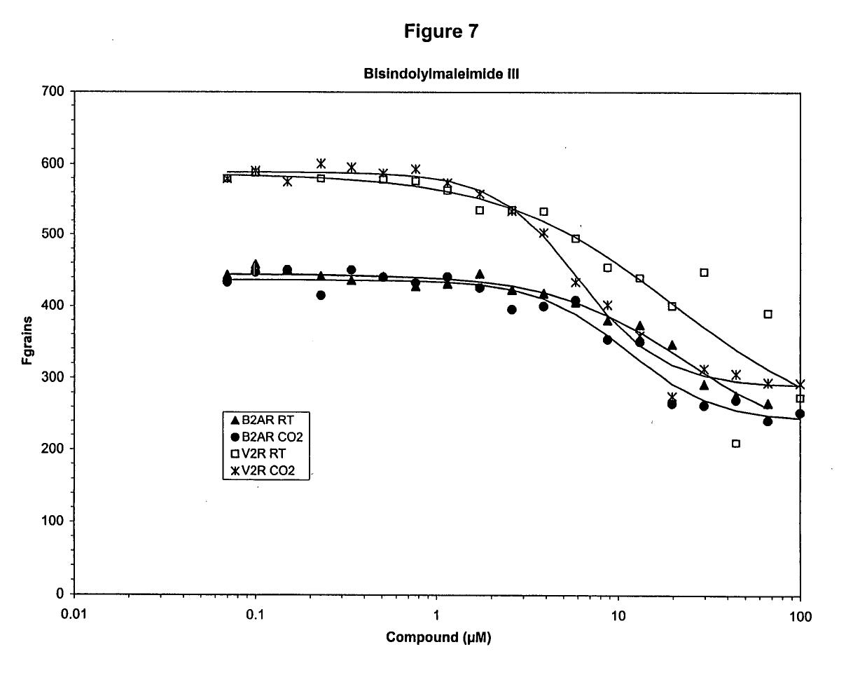

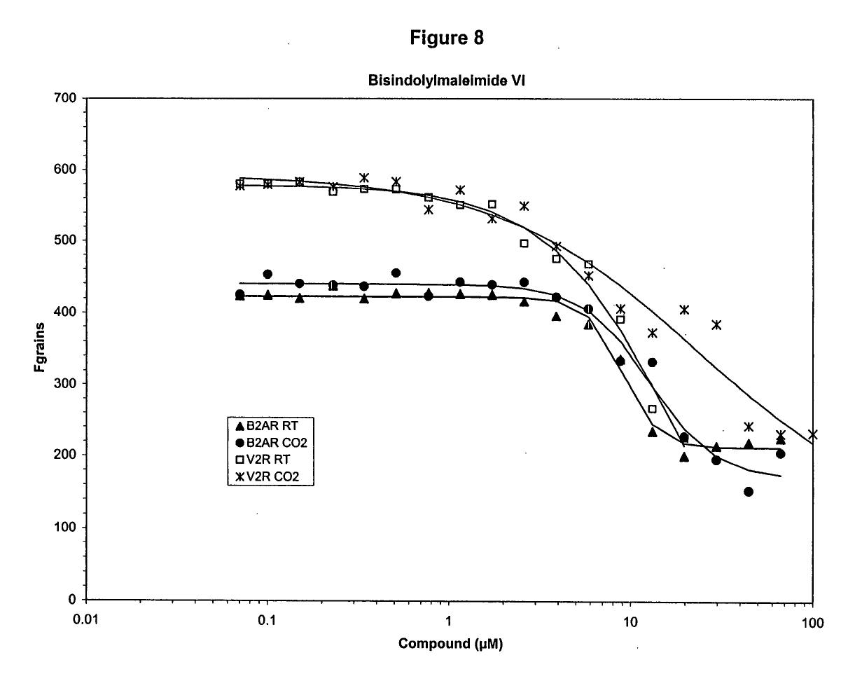

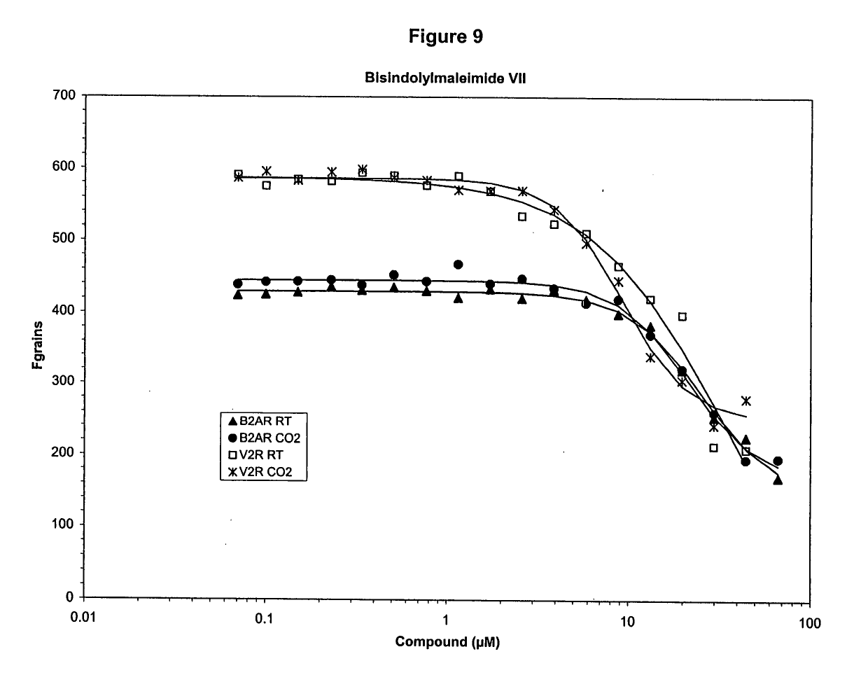

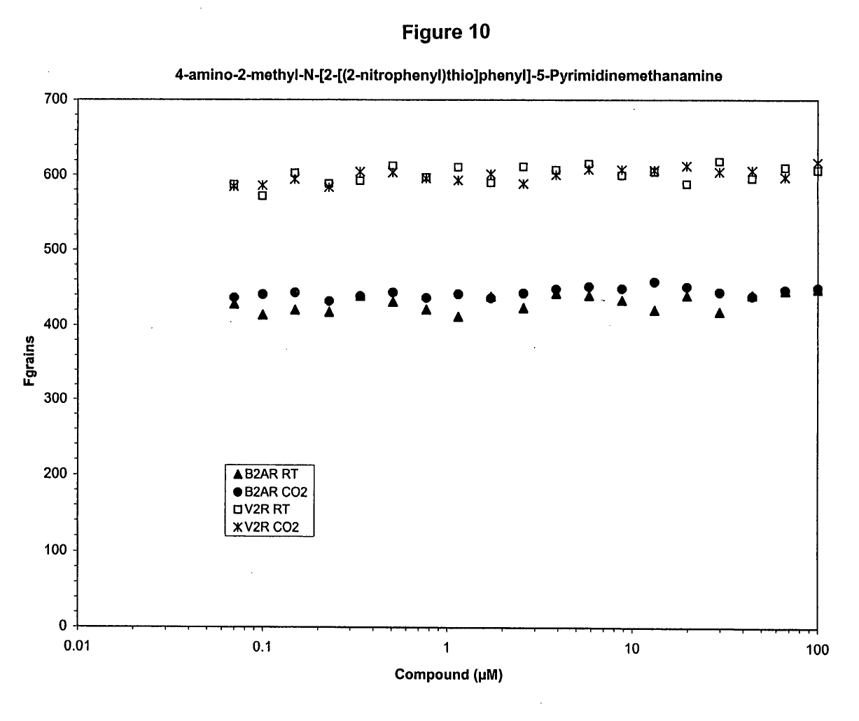

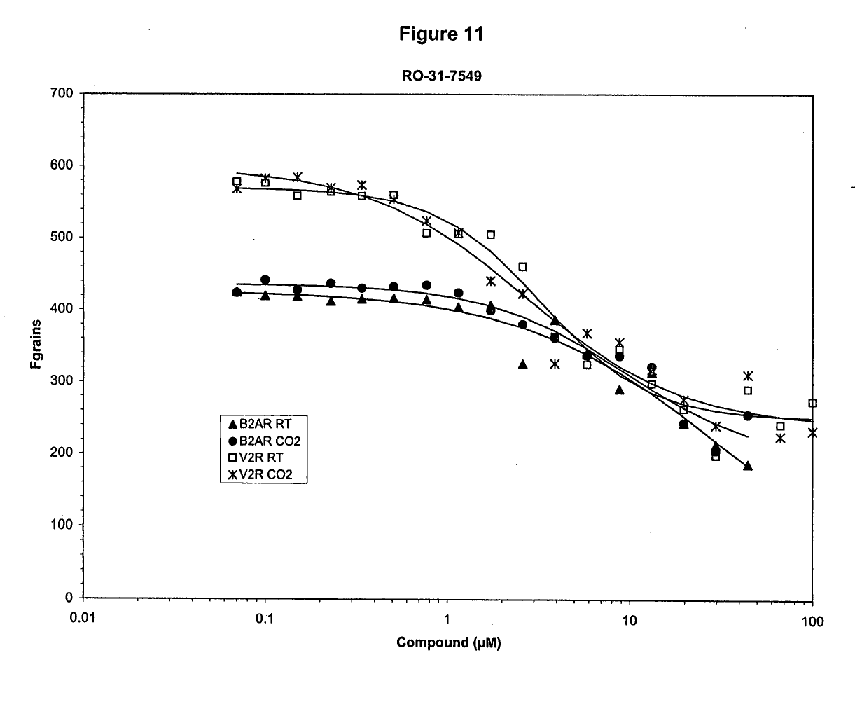

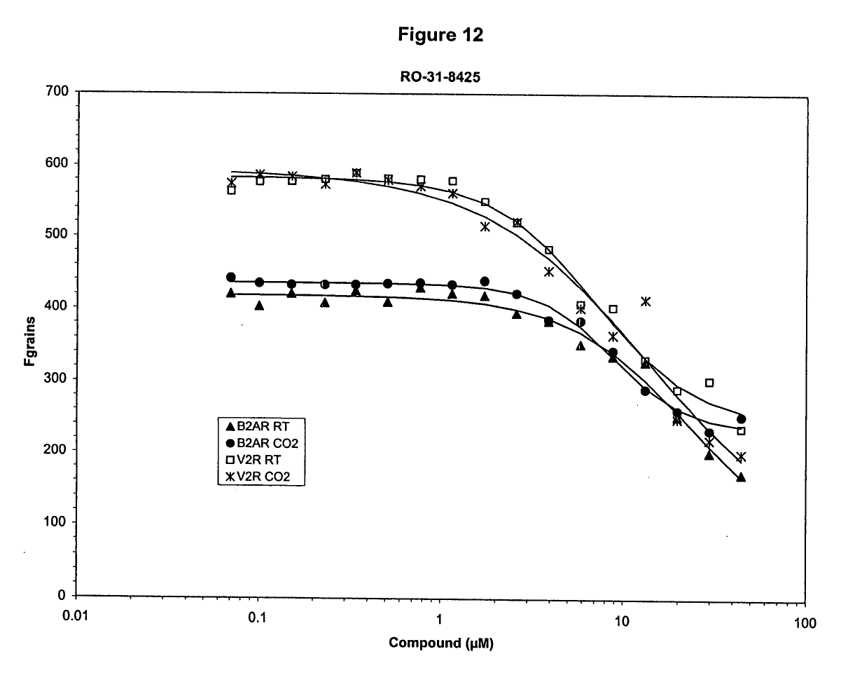

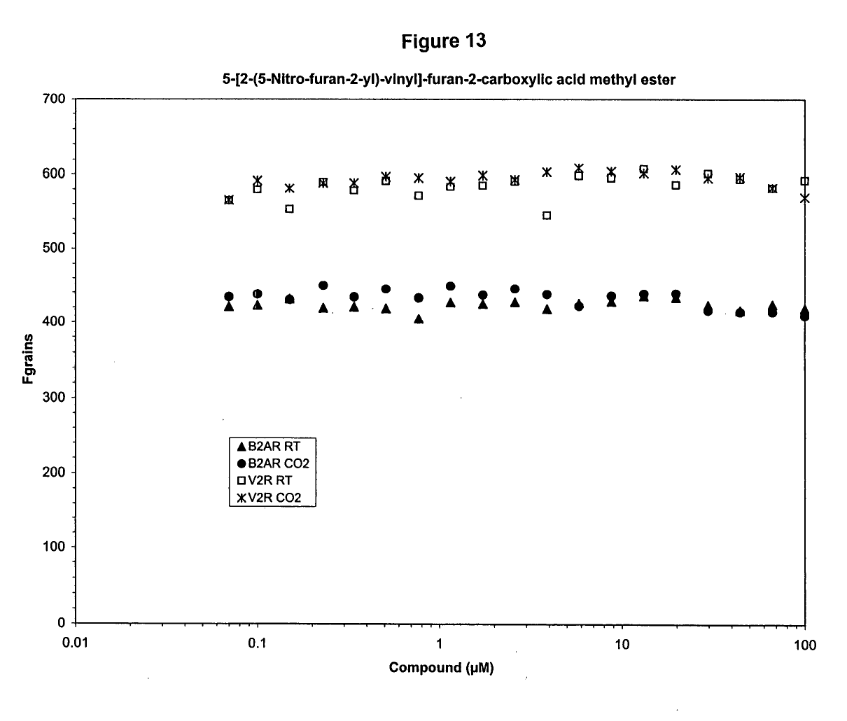

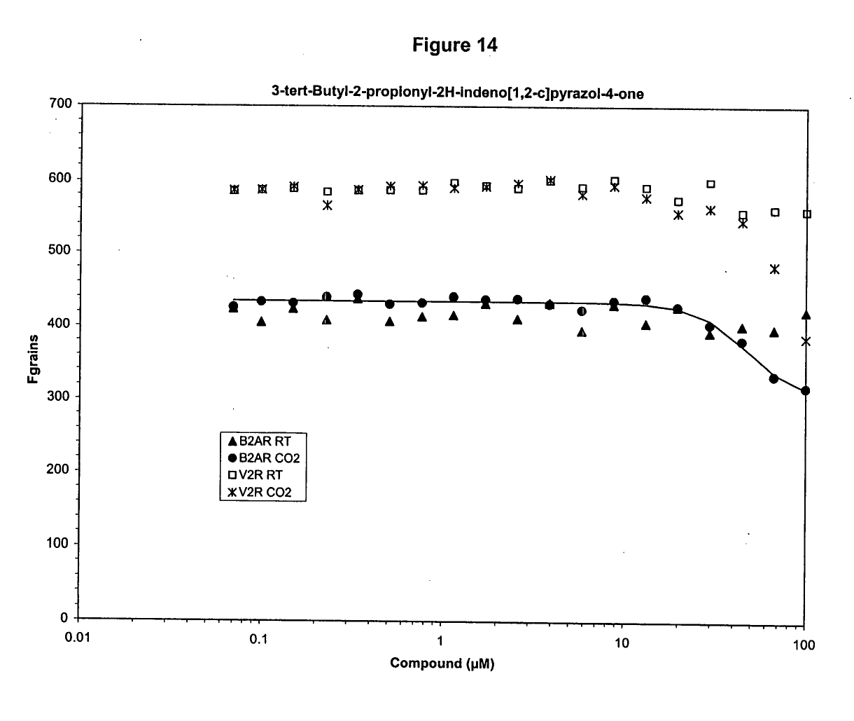

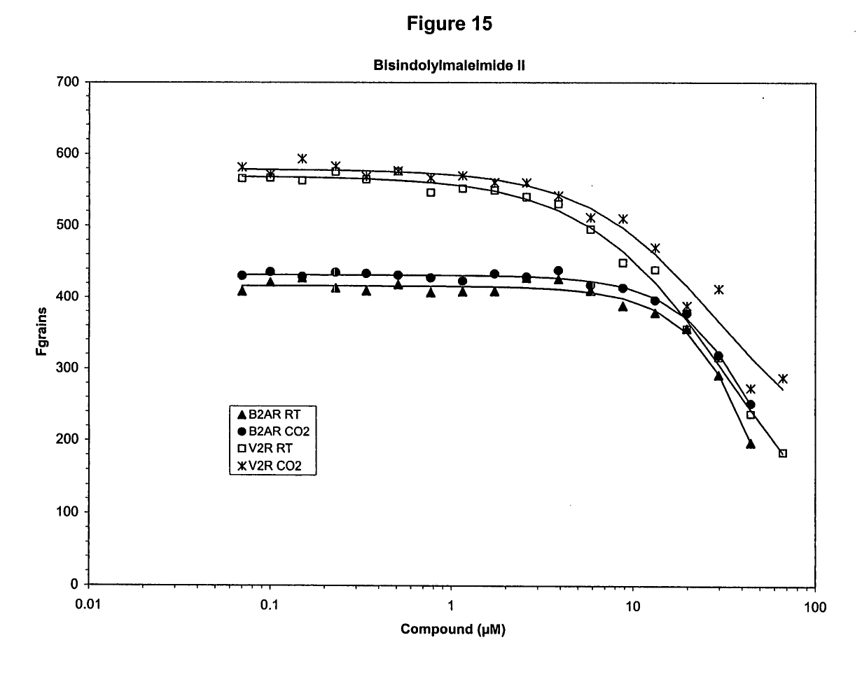

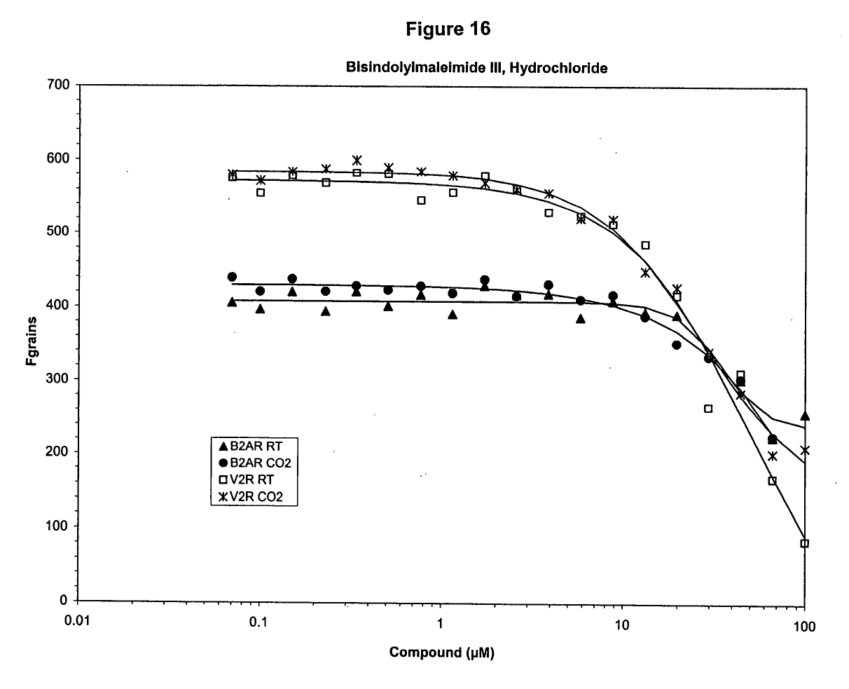

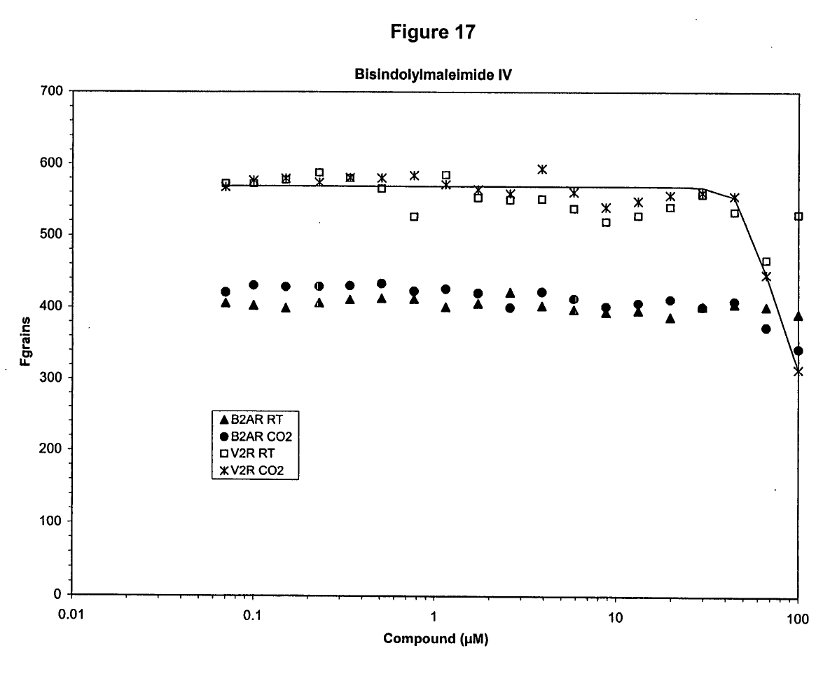

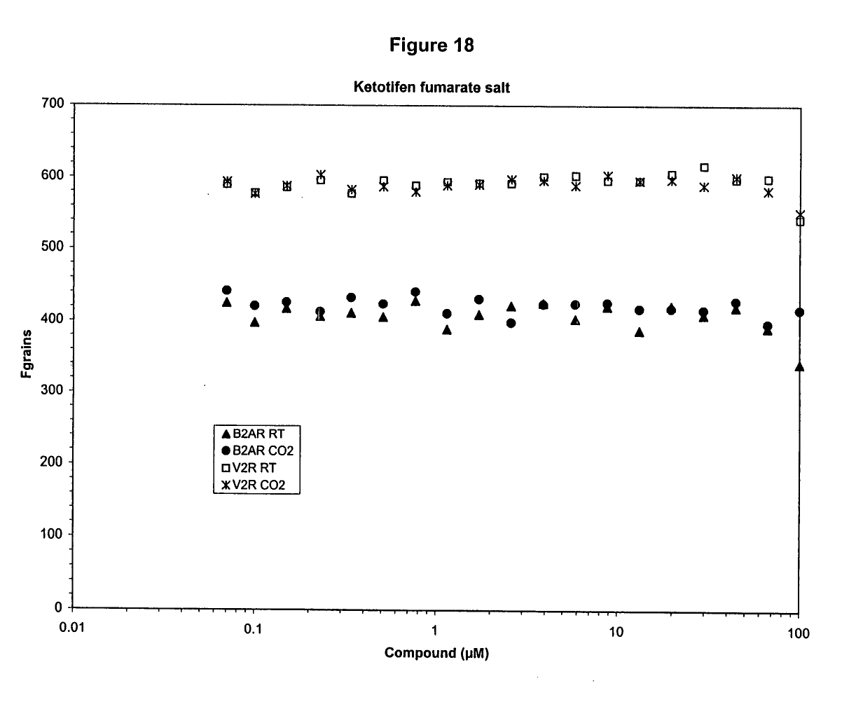

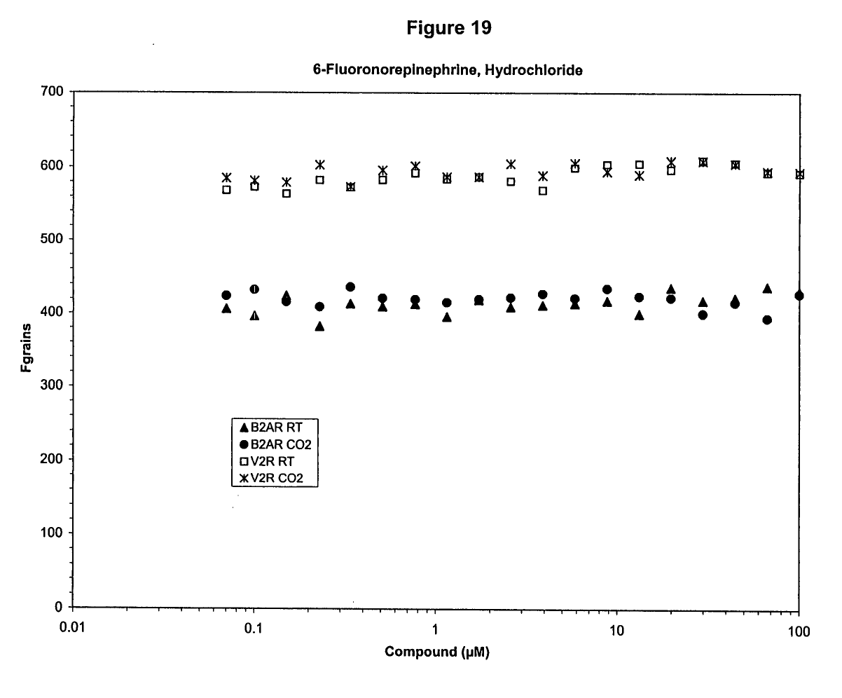

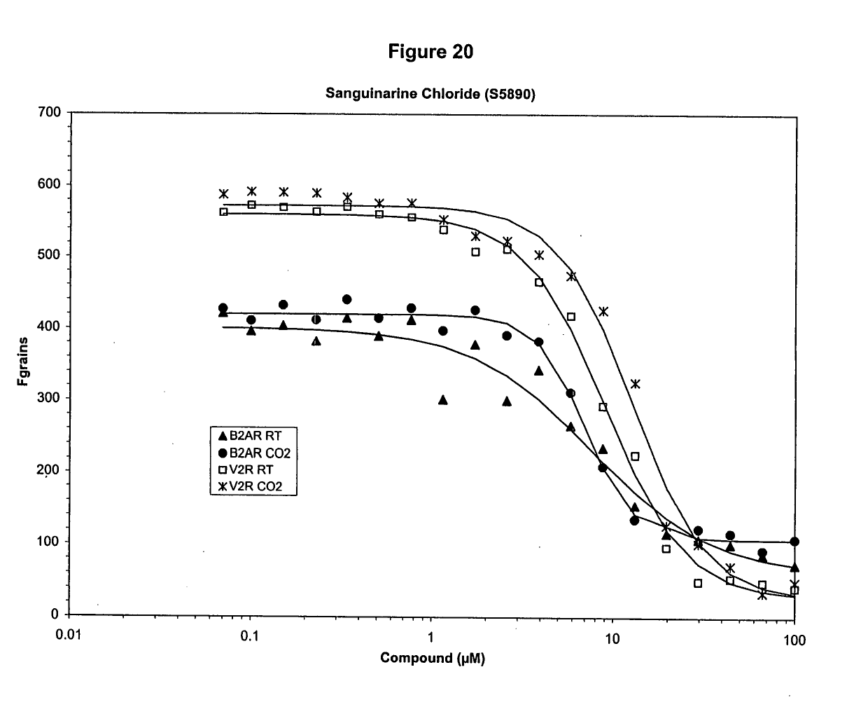

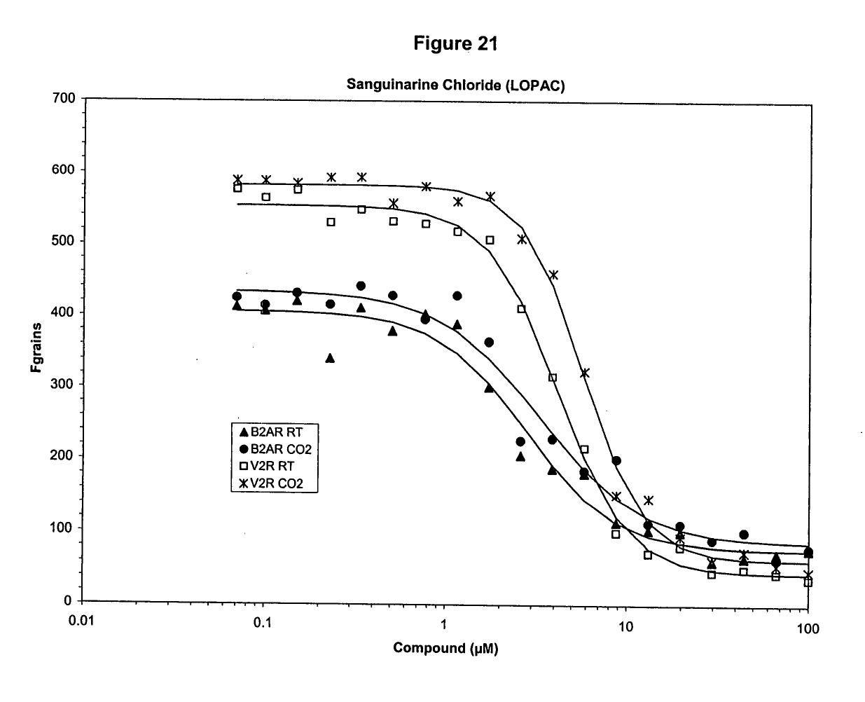

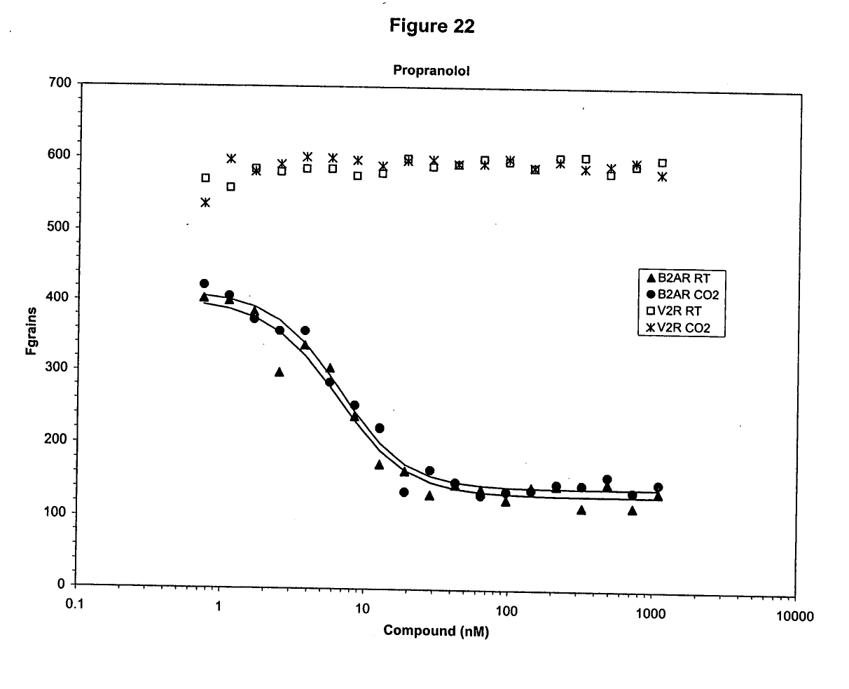

The Fgrains results for the assays of each of the test compounds and the assays for propanolol were plotted versus the concentrations of the respective compounds. Then, using a curve-fitting program, a concentration-response curve was plotted on the graph for compounds that showed inhibitory activity. No concentration-response curve was plotted on the assays that showed little or no inhibitory activity. The graphs for each compound as well as for the propanolol control are illustrated in FIGS. 7-22.

Based on the results of the concentration-response curves, the following data was also obtained for the assays in which curves were plotted: the change between the fitted maximum and fitted minimum Fgrains value for each compound (i.e., Max Rsp); the compound concentration that caused the half-maximal response (i.e., EC50); the negative log of EC50 (i.e., pEC50); the minimum Fgrains value for each compound as determined by the curve-fitting program (i.e., Min); and the slope of the calculated concentration-response curve. The curve-fitting program allowed the minimum and maximum values as well as the slope and EC50 values to vary rather than fixing the values to specified or collected values. The data obtained for each compound and the propanolol control is listed below in Tables 2-13.

TABLE 2
Bisindolylmaleimide III

| | | Max | EC50 | | | |
|---|---|---|---|---|---|---|
| Test compound | Plate | Rsp | (μM) | pEC50 | Min | slope |
| Bisindolylmaleimide III | B2AR | 232 | 22.4 | 4.7 | 212 | 1.2 |
| | RT | | | | | |
| Bisindolylmaleimide III | B2AR | 194 | 11.1 | 5.0 | 243 | 1.8 |
| | CO2 | | | | | |
| Bisindolylmaleimide III | V2R | 361 | 19.3 | 4.7 | 225 | 0.9 |
| | RT | | | | | |
| Bisindolylmaleimide III | V2R | 298 | 6.0 | 5.2 | 291 | 1.9 |
| | CO2 | | | | | |

TABLE 3
Bisindolylmaleimide VI

| | | Max | EC50 | | | |
|---|---|---|---|---|---|---|
| Test compound | Plate | Rsp | (μM) | pEC50 | Min | slope |
| Bisindolylmaleimide VI | B2AR | 210 | 9.0 | 5.0 | 213 | 4.4 |
| | RT | | | | | |
| Bisindolylmaleimide VI | B2AR | 270 | 12.7 | 4.9 | 170 | 2.4 |
| | CO2 | | | | | |
| Bisindolylmaleimide VI | V2R | 696 | 18.3 | 4.7 | −118 | 1.2 |
| | RT | | | | | |
| Bisindolylmaleimide VI | V2R | 499 | 24.6 | 4.6 | 94 | 0.8 |
| | CO2 | | | | | |

TABLE 4
Bisindolylmaleimide VII

| | | Max | EC50 | | | |
|---|---|---|---|---|---|---|
| Test compound | Plate | Rsp | (μM) | pEC50 | Min | slope |
| Bisindolylmaleimide VII | B2AR | 281 | 24.9 | 4.6 | 148 | 2.2 |
| | RT | | | | | |
| Bisindolylmaleimide VII | B2AR | 278 | 21.2 | 4.7 | 167 | 2.3 |
| | CO2 | | | | | |
| Bisindolylmaleimide VII | V2R | 717 | 36.6 | 4.4 | −130 | 1.1 |
| | RT | | | | | |
| Bisindolylmaleimide VII | V2R | 336 | 9.3 | 5.0 | 250 | 2.4 |
| | CO2 | | | | | |

TABLE 5
RO-31-7549

| | | Max | EC50 | | | |
|---|---|---|---|---|---|---|
| Test compound | Plate | Rsp | (μM) | pEC50 | Min | slope |
| RO-31-7549 | B2AR RT | 423 | 31.1 | 4.5 | 4 | 0.8 |
| RO-31-7549 | B2AR CO2 | 243 | 9.4 | 5.0 | 193 | 1.2 |
| RO-31-7549 | V2R RT | 320 | 3.3 | 5.5 | 250 | 1.5 |
| RO-31-7549 | V2R CO2 | 357 | 2.7 | 5.6 | 240 | 1.0 |

TABLE 6
RO-31-8425

| | | Max | EC50 | | | |
|---|---|---|---|---|---|---|
| Test compound | Plate | Rsp | (μM) | pEC50 | Min | slope |
| RO-31-8425 | B2AR RT | 359 | 23.2 | 4.6 | 60 | 1.3 |
| RO-31-8425 | B2AR CO2 | 203 | 8.8 | 5.1 | 233 | 2.1 |
| RO-31-8425 | V2R RT | 342 | 6.9 | 5.2 | 241 | 1.5 |
| RO-31-8425 | V2R CO2 | 532 | 13.9 | 4.9 | 60 | 1.0 |

TABLE 7
3-tert-Butyl-2-propionyl-2H-indeno[1,2-c]pyrazol-4-one

| | | Max | EC50 | | | |
|---|---|---|---|---|---|---|
| Test compound | Plate | Rsp | (μM) | pEC50 | Min | slope |
| 3-tert-Butyl-2-propionyl- | B2AR | 129 | 46.9 | 4.3 | 307 | 3.2 |
| 2H-indeno[1,2- | CO2 | | | | | |
| c]pyrazol-4-one | | | | | | |

TABLE 8
Bisindolylmaleimide II

| | | Max | EC50 | | | |
|---|---|---|---|---|---|---|
| Test compound | Plate | Rsp | (μM) | pEC50 | Min | slope |
| Bisindolylmaleimide II | B2AR RT | 924 | 87.3 | 4.1 | −508 | 1.7 |
| Bisindolylmaleimide II | B2AR | 502 | 62.1 | 4.2 | −70 | 1.7 |
| | CO2 | | | | | |
| Bisindolylmaleimide II | V2R RT | 576 | 35.0 | 4.5 | −7 | 1.1 |
| Bisindolylmaleimide II | V2R CO2 | 420 | 29.2 | 4.5 | 159 | 1.2 |

TABLE 9
Bisindolylmaleimide III, Hydrochloride

| | | Max | EC50 | | | |
|---|---|---|---|---|---|---|
| Test compound | Plate | Rsp | (μM) | pEC50 | Min | slope |
| Bisindolylmaleimide III, | B2AR | 171 | 34.9 | 4.5 | 237 | 3.5 |
| Hydrochloride | RT | | | | | |
| Bisindolylmaleimide III, | B2AR | 998 | 232.6 | 3.6 | −568 | 1.1 |
| Hydrochloride | CO2 | | | | | |
| Bisindolylmaleimide III, | V2R | 690 | 51.2 | 4.3 | −118 | 1.2 |
| Hydrochloride | RT | | | | | |
| Bisindolylmaleimide III, | V2R | 455 | 27.2 | 4.6 | 129 | 1.4 |
| Hydrochloride | CO2 | | | | | |

TABLE 10
Bisindolylmaleimide IV

| | | Max | EC50 | | | |
|---|---|---|---|---|---|---|
| Test compound | Plate | Rsp | (μM) | pEC50 | Min | slope |
| Bisindolylmaleimide IV | V2R | 277 | 69.2 | 4.2 | 292 | 6.5 |
| | CO2 | | | | | |

TABLE 11
Sanguinarine Chloride (S5890)

| | | Max | EC50 | | | |
|---|---|---|---|---|---|---|
| Test compound | Plate | Rsp | (μM) | pEC50 | Min | slope |
| Sanguinarine Chloride | B2AR RT | 340 | 7.7 | 5.1 | 61 | 1.3 |
| (S5890) | | | | | | |
| Sanguinarine Chloride | B2AR | 312 | 7.0 | 5.2 | 108 | 3.3 |
| (S5890) | CO2 | | | | | |
| Sanguinarine Chloride | V2R RT | 533 | 9.0 | 5.0 | 27 | 2.0 |
| (S5890) | | | | | | |
| Sanguinarine Chloride | V2R CO2 | 546 | 12.7 | 4.9 | 27 | 2.1 |
| (S5890) | | | | | | |

TABLE 12
Sanguinarine Chloride (LOPAC)

| | | Max | EC50 | | | |
|---|---|---|---|---|---|---|
| Test compound | Plate | Rsp | (μM) | pEC50 | Min | slope |
| Sanguinarine Chloride | B2AR RT | 330 | 2.8 | 5.6 | 76 | 1.8 |
| (LOPAC) | | | | | | |
| Sanguinarine Chloride | B2AR | 350 | 3.3 | 5.5 | 85 | 1.6 |
| (LOPAC) | CO2 | | | | | |
| Sanguinarine Chloride | V2R RT | 508 | 4.1 | 5.4 | 45 | 2.3 |
| (LOPAC) | | | | | | |
| Sanguinarine Chloride | V2R CO2 | 519 | 5.7 | 5.2 | 63 | 2.6 |
| (LOPAC) | | | | | | |

TABLE 13
Propanolol

| | | Max | EC50 | | | |
|---|---|---|---|---|---|---|
| Antagonist | Plate | Rsp | (nM) | pEC50 | Min | slope |
| Propranolol | B2AR RT | 270 | 6.3 | 8.2 | 131 | 1.8 |
| Propranolol | B2AR CO2 | 270 | 6.5 | 8.2 | 141 | 1.9 |

Table 14 below lists the mean Fgrains value for the cells in the Basal and Stimulated control wells on each plate and the standard error of the mean (S.E.M.) of each value.

TABLE 14
Basal and Stimulated Control Wells

| Plate | Basal | S.E.M. | Stimulated | S.E.M. |
|---|---|---|---|---|
| B2AR RT | 118 | 7 | 406 | 5 |
| B2AR CO2 | 139 | 5 | 431 | 1 |
| V2R RT | 77 | 6 | 568 | 1 |
| V2R CO2 | 81 | 5 | 572 | 1 |

As indicated by the Fgrains values for the cells in the Basal and Stimulated control wells, the Fgrains value determined by the IN Cell Analyzer 3000 in this example was functionally a measure of the average extent of desensitization in the cells in each well. By comparing the Fgrains level of the cells in the control wells for each plate (especially the Stimulated control) to the concentration-response curves of the test compounds in the two stable cell lines expressing different receptors, several compounds show an indication of GPCR desensitization inhibitory activity for one or both receptors. Those test compounds showing GPCR desensitization inhibitory activity for both receptors therefore also show an indication that the test compounds have non-receptor-specific GPCR desensitization inhibitory activity (i.e., Bisindolylmaleimide III; Bisindolylmaleimide VI; Bisindolylmaleimide VII; RO-31-7549; RO-31-8425; Bisindolylmaleimide II; Bisindolylmaleimide III, Hydrochloride; Sanguinarine Chloride (S5890); and Sanguinarine Chloride (LOPAC)). The propanolol control illustrated an indication of GPCR desensitization inhibitory activity with respect to the β₂AR but not the V2R.

While the invention has been described in detail and with reference to specific embodiments thereof, it will be apparent to one skilled in the art that various changes and modifications can be made without departing from the spirit and scope of the invention.

1. A method of screening a composition for non-receptor-specific G-protein coupled receptor (GPCR) desensitization inhibitory activity comprising the steps of:
(a) providing a first cell comprising a first GPCR and a first conjugate of a marker molecule and a protein associated with the desensitization pathway of the first GPCR, the first GPCR being a GPCR that requires an agonist for desensitization or being a constitutively desensitized GPCR;
(b) exposing the first cell to a test composition and, when the first GPCR requires agonist for desensitization, to an agonist for the first GPCR;
(c) determining, through detection of the marker molecule in the first conjugate, whether or not the composition gives an indication of GPCR desensitization inhibitory activity with respect to the first GPCR;
(d) providing a second cell comprising a second GPCR different from the first GPCR and a second conjugate of a marker molecule and a protein associated with the desensitization pathway of the second GPCR, the second conjugate being the same or different from the first conjugate, the second GPCR being a GPCR that requires an agonist for desensitization or being a constitutively desensitized GPCR;
(e) exposing the second cell to the test composition and, when the second GPCR requires agonist for desensitization, to an agonist for the second GPCR;
(f) determining, through detection of the marker molecule in the second conjugate, whether or not the composition gives an indication of GPCR desensitization inhibitory activity with respect to the second GPCR;
wherein an indication of GPCR desensitization inhibitory activity for the test composition in both step (c) and step (f) being an indication that the test composition has non-receptor-specific GPCR desensitization inhibitory activity.

2. The method of claim 1, wherein the protein in the first conjugate associated with the desensitization pathway of the first GPCR is selected from the group consisting of an arrestin protein and the first GPCR and wherein the protein in the second conjugate associated with the desensitization pathway of the second GPCR is selected from the group consisting of an arrestin protein and the second GPCR.

3. The method of claim 1, wherein the marker molecule of the first conjugate and the marker molecule of the second conjugate are independently selected from the group consisting of radioisotope, epitope tag, affinity label, enzyme, fluorescent group, and chemiluminescent group.

4. The method of claim 2, wherein the marker molecule of the first conjugate and the marker molecule of the second conjugate are independently selected from the group consisting of radioisotope, epitope tag, affinity label, enzyme, fluorescent group, and chemiluminescent group.

5. The method of claim 1, wherein:
step (c) comprises detecting for translocation or localization of the first conjugate, a lack of translocation or localization being an indication that the composition has GPCR desensitization inhibitory activity; and
step (f) comprises detecting for translocation or localization of the second conjugate, a lack of translocation or localization being an indication that the composition has GPCR desensitization inhibitory activity.

6. The method of claim 1, wherein:
step (c) comprises detecting for translocation or localization of the first conjugate, a decrease in translocation or localization after exposure to the test composition being an indication that the composition has GPCR desensitization inhibitory activity; and
step (f) comprises detecting for translocation or localization of the second conjugate, a decrease in translocation or localization after exposure to the test composition being an indication that the composition has GPCR desensitization inhibitory activity.

7. The method of claim 1, wherein:
step (c) comprises detecting for translocation or localization of the first conjugate, a decreased level of translocation or localization with respect to a level of translocation or localization determined in a control cell not exposed to the test composition being an indication that the composition has GPCR desensitization inhibitory activity, the control cell comprising the first GPCR and the first conjugate and being exposed to agonist if the first GPCR requires agonist for desensitization; and
step (f) comprises detecting for translocation or localization of the second conjugate, a decreased level of translocation or localization with respect to a level of translocation or localization determined in a control cell not exposed to the test composition being an indication that the composition has GPCR desensitization inhibitory activity, the control cell comprising the second GPCR and the second conjugate and being exposed to agonist if the second GPCR requires agonist for desensitization.

8. The method of claim 1, wherein:
the first GPCR is a GPCR that requires agonist for desensitization;
in step (b), the first cell is exposed to the test composition first and then is exposed to the agonist for the first GPCR;
the second GPCR is a GPCR that requires agonist for desensitization; and
in step (e), the second cell is exposed to the test composition first and then is exposed to the agonist for the second GPCR.

9. The method of claim 1, wherein:
the first GPCR is a GPCR that requires agonist for desensitization;
in step (b), the first cell is exposed to the agonist for the first GPCR and then is exposed to the test composition;
the second GPCR is a GPCR that requires agonist for desensitization; and
in step (e), the second cell is exposed to the agonist for the second GPCR and then is exposed to the test composition.

10. The method of claim 1, wherein:
in the first conjugate, the protein associated with the desensitization pathway of the first GPCR comprises an arrestin protein and the marker molecule comprises a GFP;
in the second conjugate, the protein associated with the desensitization pathway of the second GPCR comprises an arrestin protein and the marker molecule comprises a GFP;
the first GPCR is a GPCR that requires agonist for desensitization;
the second GPCR is a GPCR that requires agonist for desensitization;
in step (b), the first cell is exposed to the test composition first and then is exposed to the agonist for the first GPCR;
step (c) comprises detecting for translocation or localization of the first conjugate, a lack of translocation or localization being an indication that the composition has GPCR desensitization inhibitory activity;
in step (e), the second cell is exposed to the test composition first and then is exposed to the agonist for the second GPCR; and
step (f) comprises detecting for translocation or localization of the second conjugate, a lack of translocation or localization being an indication that the composition has GPCR desensitization inhibitory activity.

11. The method of claim 1, wherein:
the first conjugate comprises the first GPCR conjugated to the marker molecule;
the second conjugate comprises the second GPCR conjugated to the marker molecule;
step (c) comprises detecting for translocation or localization of the first GPCR, a lack of translocation or localization being an indication that the composition has GPCR desensitization inhibitory activity; and
step (f) comprises detecting for translocation or localization of the second GPCR, a lack of translocation or localization being an indication that the composition has GPCR desensitization inhibitory activity.

12. The method of claim 1, wherein:
the first conjugate comprises the first GPCR conjugated to the marker molecule;
the second conjugate comprises the second GPCR conjugated to the marker molecule;
step (c) comprises detecting for translocation or localization of the first GPCR, a decrease in translocation or localization after exposure to the test composition being an indication that the composition has GPCR desensitization inhibitory activity; and
step (f) comprises detecting for translocation or localization of the second GPCR, a decrease in translocation or localization after exposure to the test composition being an indication that the composition has GPCR desensitization inhibitory activity.

13. A method of screening a composition for non-receptor-specific G-protein coupled receptor (GPCR) desensitization inhibitory activity comprising the steps of:
(a) providing a first cell comprising a first GPCR that requires agonist for desensitization and a first conjugate of a marker molecule and a protein associated with the desensitization pathway of the first GPCR;
(b) exposing the first cell to a test composition and to an agonist for the first GPCR;
(c) determining whether or not the composition gives an indication of GPCR desensitization inhibitory activity with respect to the first GPCR by detecting for translocation or localization of the first conjugate, the following being an indication that the composition has GPCR desensitization inhibitory activity:
(1) a lack of translocation or localization;
(2) a decrease in translocation or localization after exposure to the test composition; or
(3) a decreased level of translocation or localization with respect to a predetermined level or with respect to a level determined in a control cell not exposed to the test composition, the control cell comprising the first GPCR and the first conjugate and being exposed to the agonist for the first GPCR;
(d) providing a second cell comprising a second GPCR that requires agonist for desensitization and is different from the first GPCR and a second conjugate of a marker molecule and a protein associated with the desensitization pathway of the second GPCR, the second conjugate being the same or different from the first conjugate;
(e) exposing the second cell to the test composition and to an agonist for the second GPCR;
(f) determining whether or not the composition gives an indication of GPCR desensitization inhibitory activity with respect to the second GPCR by detecting for translocation or localization of the second conjugate, the following being an indication that the composition has GPCR desensitization inhibitory activity:
(1) a lack of translocation or localization;
(2) a decrease in translocation or localization after exposure to the test composition; or
(3) a decreased level of translocation or localization with respect to a predetermined level or with respect to a level determined in a control cell not exposed to the test composition, the control cell comprising the second GPCR and the second conjugate and being exposed to the agonist for the second GPCR;
wherein an indication of GPCR desensitization inhibitory activity for the test composition in both step (c) and step (f) being an indication that the test composition has non-receptor-specific GPCR desensitization inhibitory activity.

14. The method of claim 13, wherein the protein in the first conjugate associated with the desensitization pathway of the first GPCR is selected from the group consisting of an arrestin protein and the first GPCR and wherein the protein in the second conjugate associated with the desensitization pathway of the second GPCR is selected from the group consisting of an arrestin protein and the second GPCR.

15. The method of claim 14, wherein the marker molecule of the first conjugate and the marker molecule of the second conjugate are independently selected from the group consisting of radioisotope, epitope tag, affinity label, enzyme, fluorescent group, and chemiluminescent group.

16. The method of claim 15, wherein:
in the first conjugate, the protein associated with the desensitization pathway of the first GPCR comprises an arrestin protein and the marker molecule comprises a GFP; and
in the second conjugate, the protein associated with the desensitization pathway of the second GPCR comprises an arrestin protein and the marker molecule comprises a GFP.

17. The method of claim 16, wherein, when there is an indication that the test composition has non-receptor-specific GPCR desensitization inhibitory activity, the method further comprises performing a kinase assay to determine whether the test composition is a kinase inhibitor.

18. The method of claim 17, wherein, when there is an indication that the test composition has non-receptor-specific GPCR desensitization inhibitory activity, the method further comprises performing a GPCR ligand binding assay to confirm that the GPCR desensitization inhibitory activity of the test composition is not receptor specific.

19. A method of screening a composition for non-receptor-specific G-protein coupled receptor (GPCR) desensitization inhibitory activity comprising the steps of:
(a) providing a cell comprising:
(1) a first GPCR that is a GPCR that requires agonist for desensitization;
(2) a second GPCR that is different than the first GPCR, the second GPCR being a GPCR that requires agonist for desensitization;
(3) a first conjugate of a marker molecule and a protein associated with the desensitization pathway of the first GPCR; and
(4) a second conjugate of a marker molecule and a protein associated with the desensitization pathway of the second GPCR, the second conjugate being the same or different from the first conjugate;
(b) exposing the cell to a test composition and to an agonist for the first GPCR;
(c) determining whether or not the composition has GPCR desensitization inhibitory activity with respect to the first GPCR;
(d) exposing the cell to an agonist for the second GPCR and optionally re-exposing the cell to the test composition; and
(e) determining whether or not the composition has GPCR desensitization inhibitory activity with respect to the second GPCR;
wherein an indication that the test composition has GPCR desensitization inhibitory activity with respect to both the first GPCR and the second GPCR being an indication that the test composition has non-receptor-specific GPCR desensitization inhibitory activity; and
wherein the agonist for the first GPCR is not an agonist for the second GPCR and the agonist for the second GPCR is not an agonist for the first GPCR.

20. The method of claim 19, wherein the protein in the first conjugate associated with the desensitization pathway of the first GPCR is selected from the group consisting of an arrestin protein and the first GPCR and wherein the protein in the second conjugate associated with the desensitization pathway of the second GPCR is selected from the group consisting of an arrestin protein and the second GPCR.

21. The method of claim 19, wherein the marker molecule of the first conjugate and the marker molecule of the second conjugate are independently selected from the group consisting of radioisotope, epitope tag, affinity label, enzyme, fluorescent group, and chemiluminescent group.

22. The method of claim 20, wherein the marker molecule of the first conjugate and the marker molecule of the second conjugate are independently selected from the group consisting of radioisotope, epitope tag, affinity label, enzyme, fluorescent group, and chemiluminescent group.

23. The method of claim 19, wherein:
step (c) comprises detecting for translocation or localization of the conjugate, a lack of translocation or localization being an indication that the composition has GPCR desensitization inhibitory activity with respect to the first GPCR; and
step (e) comprises detecting for translocation or localization of the conjugate, a lack of translocation or localization being an indication that the composition has GPCR desensitization inhibitory activity with respect to the second GPCR.

24. The method of claim 19, wherein:
step (c) comprises detecting for translocation or localization of the conjugate, a decrease in translocation or localization after exposure to the test composition being an indication that the composition has GPCR desensitization inhibitory activity with respect to the first GPCR; and
step (e) comprises detecting for translocation or localization of the second conjugate, a decrease in translocation or localization after exposure to the test composition being an indication that the composition has GPCR desensitization inhibitory activity with respect to the second GPCR.

25. The method of claim 19, wherein:
step (c) comprises detecting for translocation or localization of the conjugate, a decreased level of translocation or localization with respect to a level of translocation or localization determined in a control cell not exposed to the test composition being an indication that the composition has GPCR desensitization inhibitory activity with respect to the first GPCR, the control cell comprising the first GPCR and the conjugate and being exposed to agonist if the first GPCR requires agonist for desensitization; and
step (e) comprises detecting for translocation or localization of the conjugate, a decreased level of translocation or localization with respect to a level of translocation or localization determined in a control cell not exposed to the test composition being an indication that the composition has GPCR desensitization inhibitory activity with respect to the second GPCR, the control cell comprising the second GPCR and the conjugate and being exposed to agonist if the second GPCR requires agonist for desensitization.

26. A method of screening a composition for non-receptor-specific G-protein coupled receptor (GPCR) desensitization inhibitory activity comprising the steps of:
(a) providing a cell comprising:
(1) a first GPCR that is a GPCR that requires agonist for desensitization or is a constitutively desensitized GPCR;
(2) a second GPCR that is different than the first GPCR, the second GPCR being a GPCR that requires agonist for desensitization or being a constitutively desensitized GPCR;
(3) a first conjugate of a first marker molecule and a protein associated with the desensitization pathway of the first GPCR; and
(4) a second conjugate of a second marker molecule and a protein associated with the desensitization pathway of the second GPCR, the second conjugate being different from the first conjugate;
wherein the protein of the first conjugate is not included in the desensitization pathway of the second GPCR, the protein in the second conjugate is not included in the desensitization pathway of the first GPCR, and the first and second marker molecules are different from each other and are distinguishable from each other upon detection;
(b) exposing the cell (1) to a test composition, (2) when the first GPCR requires agonist for desensitization, to an agonist for the first GPCR, and (3) when the second GPCR requires agonist for desensitization, to an agonist for the second GPCR; and
(c) determining whether or not the composition has GPCR desensitization inhibitory activity with respect to the first GPCR and with respect to the second GPCR, wherein an indication that the test composition has GPCR desensitization inhibitory activity with respect to both the first GPCR and the second GPCR being an indication that the test composition has non-receptor-specific GPCR desensitization inhibitory activity.

27. The method of claim 26, wherein:
the first conjugate comprises the first GPCR conjugated to the first marker molecule; and
the second conjugate comprises the second GPCR conjugated to the second marker molecule.

28. The method of claim 26, wherein the marker molecule of the first conjugate and the marker molecule of the second conjugate are independently selected from the group consisting of radioisotope, epitope tag, affinity label, enzyme, fluorescent group, and chemiluminescent group.

29. The method of claim 27, wherein the marker molecule of the first conjugate and the marker molecule of the second conjugate are independently selected from the group consisting of radioisotope, epitope tag, affinity label, enzyme, fluorescent group, and chemiluminescent group.

30. The method of claim 26, wherein step (c) comprises:
(1) detecting for translocation or localization of the first conjugate, a lack of translocation or localization being an indication that the composition has GPCR desensitization inhibitory activity with respect to the first GPCR; and
(2) detecting for translocation or localization of the second conjugate, a lack of translocation or localization being an indication that the composition has GPCR desensitization inhibitory activity with respect to the second GPCR.

31. The method of claim 26, wherein step (c) comprises:
(1) detecting for translocation or localization of the first conjugate, a decrease in translocation or localization after exposure to the test composition being an indication that the composition has GPCR desensitization inhibitory activity with respect to the first GPCR; and
(2) detecting for translocation or localization of the second conjugate, a decrease in translocation or localization after exposure to the test composition being an indication that the composition has GPCR desensitization inhibitory activity with respect to the second GPCR.

32. The method of claim 26, wherein step (c) comprises:
(1) detecting for translocation or localization of the first conjugate, a decreased level of translocation or localization with respect to a level of translocation or localization determined in a control cell not exposed to the test composition being an indication that the composition has GPCR desensitization inhibitory activity with respect to the first GPCR, the control cell comprising the first GPCR and the first conjugate and being exposed to agonist if the first GPCR requires agonist for desensitization; and
(2) detecting for translocation or localization of the second conjugate, a decreased level of translocation or localization with respect to a level of translocation or localization determined in a control cell not exposed to the test composition being an indication that the composition has GPCR desensitization inhibitory activity with respect to the second GPCR, the control cell comprising the second GPCR and the second conjugate and being exposed to agonist if the second GPCR requires agonist for desensitization.

33. The method of claim 26, wherein:
the first GPCR is a GPCR that requires agonist for desensitization;
the second GPCR is a GPCR that requires agonist for desensitization; and
in step (b), the cell is exposed to the test composition before the cell is exposed to the agonist for the first GPCR or to the agonist for the second GPCR.

34. The method of claim 26, wherein:
the first conjugate comprises the first GPCR conjugated to the first marker molecule;
the second conjugate comprises the second GPCR conjugated to the second marker molecule; and
step (c) comprises:
(1) detecting for translocation or localization of the first conjugate, a lack of translocation or localization being an indication that the composition has GPCR desensitization inhibitory activity with respect to the first GPCR; and
(2) detecting for translocation or localization of the second conjugate, a lack of translocation or localization being an indication that the composition has GPCR desensitization inhibitory activity with respect to the second GPCR.

35. The method of claim 26, wherein:
the first conjugate comprises the first GPCR conjugated to the first marker molecule;
the second conjugate comprises the second GPCR conjugated to the second marker molecule; and
step (c) comprises:
(1) detecting for translocation or localization of the first conjugate, a decrease in translocation or localization after exposure to the test composition being an indication that the composition has GPCR desensitization inhibitory activity with respect to the first GPCR; and
(2) detecting for translocation or localization of the second conjugate, a decrease in translocation or localization after exposure to the test composition being an indication that the composition has GPCR desensitization inhibitory activity with respect to the second GPCR.


SEQUENCE LISTING (65 sequences)

SEQ ID NO: 1 - protein, 43 aa, Homo sapiens
NPIVYAFRIQKFRVTFLKIWNDHFRCQPAPPIDEDLPEERPDD

SEQ ID NO: 2 - protein, 177 aa, Homo sapiens
NPIIYPCSSKEFKRAFVRILGCQCRGRGRRRRRRRRRLGGCAYTYRPWTRGGSLERSQSRKDSLDDSGSCLSGSQRTLPSASPSPGYLGRGAPPPVELCAFPEWKAPGALLSLPAPEPPGRRGRHDSGPLFTFKLLTEPESPGTDGGASNGGCEAAADVANGQPGFKSNMPLAPGQF

SEQ ID NO: 3 - protein, 29 aa, Homo sapiens
NPVIYTIFNHDFRRAFKKILCRGDRKRIV

SEQ ID NO: 4 - protein, 29 aa, Homo sapiens
NPVIYTIFNQDFRRAFRRILCRPWTQTAW

SEQ ID NO: 5 - protein, 30 aa, Homo sapiens
NPVIYTVFNQDFRPSFKHILFRRRRRGFRQ

SEQ ID NO: 6 - protein, 105 aa, Homo sapiens
NPIIYCRSPDFRKAFQGLLCCARRAARRRHATHGDRPRASGCLARPGPPPSPGAASDDDDDDVVGATPPARLLEPWAGCNGGAAADSDSSLDEPCRPGFASESKV

SEQ ID NO: 7 - protein, 92 aa, Homo sapiens
NPLIYCRSPDFRIAFQELLCLRRSSLKAYGNGYSSNGNTGEQSGYHVEQEKENKLLCEDLPGTEDFVGHQGTVPSDNIDSQGRNCSTNDSLL

SEQ ID NO: 8 - protein, 120 aa, Homo sapiens
NPIIYAFNADFRKAFSTLLGCYRLCPATNNAIETVSINNNGAAMFSSHHEPRGSISKECNLVYLIPHAVGSSEDLKKEEAAGIARPLEKLSPALSVILDYDTDVSLEKIQPITQNGQHPT

SEQ ID NO: 9 - protein, 22 aa, Homo sapiens
NPIIYTTFNIEFRKAFLKILHC

SEQ ID NO: 10 - protein, 22 aa, Homo sapiens
NPVIYTTFNIEFRKAFLKILSC

SEQ ID NO: 11 - protein, 24 aa, Homo sapiens
NPVIYTVFNAEFRNVFRKALRACC

SEQ ID NO: 12 - protein, 123 aa, Homo sapiens
NPVIYAFNADFQKVFAQLLGCSHFCSRTPVETVNISNELISYNQDIVFHKEIAAAYIHMMPNAVTPGNREVDNDEEEGPFDRMFQIYQTSPDGDPVAESVWELDCEGEISLDKITPFTPNGFH

SEQ ID NO: 13 - protein, 47 aa, Homo sapiens
NPMCYALCNKAFRDTFRLLLLCRWDKRRWRKIPKRPGSVHRTPSRQC

SEQ ID NO: 14 - protein, 31 aa, Homo sapiens
NPACYALCNATFKKTFKHLLMCHYKNIGATR

SEQ ID NO: 15 - protein, 50 aa, Homo sapiens
NPVCYALCNKTFRTTKMLLLCQCDKKKRRKQQYQQRQSVIFHKRAPEQAL

SEQ ID NO: 16 - protein, 31 aa, Homo sapiens
NPACYALCNATFKKTFRHLLLCQYRNIGTAR

SEQ ID NO: 17 - protein, 42 aa, Homo sapiens
NPICYALCNRTFRKTFKMLLLCRWKKKKVEEKLYWQGNSKLP

SEQ ID NO: 18 - protein, 24 aa, Homo sapiens
NPVIYAYFNKDFQNAFKKIIKCKF

SEQ ID NO: 19 - protein, 26 aa, Homo sapiens
NPIIYTMSNEDFKQAFHKLIRFKCTS

SEQ ID NO: 20 - protein, 24 aa, Homo sapiens
NPLLYTSFNEDFKLAFKKLIRCRE

SEQ ID NO: 21 - protein, 37 aa, Homo sapiens
NPIIYCLRNQEVKRALCCILHLYQHQDPDPKKGSRNV

SEQ ID NO: 22 - protein, 27 aa, Homo sapiens
NPLIYTLRNMEVKGALRRLLGKGREVG

SEQ ID NO: 23 - protein, 62 aa, Homo sapiens
NPLFYGFLGKKFKRYFLQLLKYIPPKAKSHSNLSTKMSTLSYRPSDNVSSSTKKPAPCFEVE

SEQ ID NO: 24 - protein, 50 aa, Homo sapiens
NPFLYCFVGNRFQQKLRSVFRVPITWLQGKRESMSCRKSSSLREMETFVS

SEQ ID NO: 25 - protein, 50 aa, Homo sapiens
NPLIYAFIGQKFRHGLLKILAIHGLSKDSLPKDSRPSFVGSSSGHTSTTL

SEQ ID NO: 26 - protein, 67 aa, Homo sapiens
NPLIYAFAGEKFRRYLYHLYGKCLAVLCGRSVHVDFSSSESQRSRHGSVLSSNFTYHTSDGDALLLL

SEQ ID NO: 27 - protein, 59 aa, Homo sapiens
NPILYNLVSANFRHIFLATLACLCPVWRRRRKRPAFSRKADSVSSNHTLSSNATRETLY

SEQ ID NO: 28 - protein, 107 aa, Homo sapiens
NPIIYCCLNDRFRLGFKHAFRCCPFISAGDYEGLEMKSTRYLQTQGSVYKVSRLETTISTVVGAHEEEPEDGPKATPSSLDLTSNCSSRSDSKTMTESFSFSSNVLS

SEQ ID NO: 29 - protein, 51 aa, Homo sapiens
NPWIYASFSSSVSSELRSLLCCARGRTPPSLGPQDESCTTASSSLAKDTSS

SEQ ID NO: 30 - protein, 83 aa, Homo sapiens
NPVIYNLMSQKFRAAFRKLCNCKQKPTEKPANYSVALNYSVIKESDHFSTELDDITVTDTYLSATKVSFDDTCLASEVSFSQS

SEQ ID NO: 31 - protein, 65 aa, Homo sapiens
NPWIYMLFTGHLFHELVQRFLCCSASYLKGRRLGETSASKKSNSSSFVLSHRSSSQRSCSQPSTA

SEQ ID NO: 32 - protein, 75 aa, Homo sapiens
NPVLYSLMSSRFRETFQEALCLGACCHRLRPRHSSHSLSRMTTGSTLCDVGSLGSWVHPLAGNDGPEAQQETDPS

SEQ ID NO: 33 - protein, 62 aa, Homo sapiens
NPLVYCFMHRRFRQACLETCARCCPRPPRARPRALPDEDPPTPSIASLSRLSYTTISFLGPG

SEQ ID NO: 34 - protein, 82 aa, Homo sapiens
NPLVYALASRHFRARFRRLWPCGRRRRHRARRALRRVRPASSGPPGCPGDARPSGRLLAGGGQGPEPREGPVHGGEAARGPE

SEQ ID NO: 35 - protein, 76 aa, Homo sapiens
NPIIYTLTNKEMRRAFIRIMSCCKCPSGDSAGKFKRPIIAGMEFSRSKSDNSSHPQKDEGDNPETIMSSGNVNSSS

SEQ ID NO: 36 - protein, 80 aa, Homo sapiens
NPIIYALRSKDLRHAFRSMFPSCEGTAQPLDNSMGDSDCLHKHANNAASVHRAAESCIKSTVKIAKVTMSVSTDTSAEAL

SEQ ID NO: 37 - protein, 59 aa, Homo sapiens
NPVLYAFLDENFKRCFRQLCRKPCGRPDPSSFSRPREATARERVTACTPSDGPGGGRAA

SEQ ID NO: 38 - protein, 58 aa, Homo sapiens
DPFVYYFVSHDFRDHAKNALLCRSVRTVKQMQVSLTSKKHSRKSSSYSSSSTTVKTSY

SEQ ID NO: 39 - protein, 66 aa, Homo sapiens
NGEVQAELRRKWRRWHLQGVLGWSSKSQHPWGGSNGATCSTQVSMLTRVSPSARRSSSFQAEVSLV

SEQ ID NO: 40 - protein, 371 aa, Homo sapiens
MLMASTTSAVPGHPSLPSLPSNSSQERPLDTRDPLLARAELALLSIVFVAVALSNGLVLAALARRGRRGHWAPIHVFIGHLCLADLAVALFQVLPQLAWKATDRFRGPDALCRAVKYLQMVGMYASSYMILAMTLDRHRAICRPMLAYRHGSGAHWNRPVLVAWAFSLLLSLPQLFIFAQRNVEGGSGVTDCWACFAEPWGRRTYVTWIALMVFVAPTLGIAACQVLIFREIHASLVPGPSERPGGRRRGRRTGSPGEGAHVSAAVAKTVRMTLVIVVVYVLCWAPFFLVQLWAAWDPEAPLEGAPFVLLMLLASLNSCTNPWIYASFSSSVSSELRSLLCCARGRTPPSLGPQDESCTTASSSLAKDTSS

SEQ ID NO: 41 - protein, 515 aa, Golden hamster
MNPDLDTGHNTSAPAQWGELKDANFTGPNQTSSNSTLPQLDVTRAISVGLVLGAFILFAIVGNILVILSVACNRHLRTPTNYFIVNLAIADLLLSFTVLPFSATLEVLGYWVLGRIFCDIWAAVDVLCCTASILSLCAISIDEYIGVRYSLQYPTLVTRRKAILALLSVWVLSTVISIGPLLGWKEPAPNDDKECGVTEEPFYALFSSLGSFYIPLAVILVMYCRVYIVAKRTTKNLEAGVMKEMSNSKELTLRIHSKNFHEDTLSSTKAKGHNPRSSIAVKLFKFSREKKAAKTLGIVVGMFILCWLPFFIALPLGSLFSTLKPPDAVFKVVFWLGYFNSCLNPIIYPCSSKEFKRAFMRILGCQCRSGRRRRRRRRLGACAYTYRPWTRGGSLERSQSRKDSLDDSGSCMSGSQRTLPSASPSPGYLGRGAQPPLELCAYPEWKSGALLSLPEPPGRRGRLDSGPLFTFKLLGEPESPGTEGDASNGGCDATTDLANGQPGFKSNMPLAPGHF

SEQ ID NO: 42 - protein, 515 aa, Golden hamster
MNPDLDTGHNTSAPAQWGELKDANFTGPNQTSSNSTLPQLDVTRAISVGLVLGAFILFAIVGNILVILSVACNRHLRTPTNYFIVNLAIADLLLSFTVLPFSATLEVLGYWVLGRIFCDIWAAVDVLCCTASILSLCAISIDAYIGVRYSLQYPTLVTRRKAILALLSVWVLSTVISIGPLLGWKEPAPNDDKECGVTEEPFYALFSSLGSFYIPLAVILVMYCRVYIVAKRTTKNLEAGVMKEMSNSKELTLRIHSKNFHEDTLSSTKAKGHNPRSSIAVKLFKFSREKKAAKTLGIVVGMFILCWLPFFIALPLGSLFSTLKPPDAVFKVVFWLGYFNSCLNPIIYPCSSKEFKRAFMRILGCQCRSGRRRRRRRRLGACAYTYRPWTRGGSLERSQSRKDSLDDSGSCMSGSQRTLPSASPSPGYLGRGAQPPLELCAYPEWKSGALLSLPEPPGRRGRLDSGPLFTFKLLGEPESPGTEGDASNGGCDATTDLANGQPGFKSNMPLAPGHF

SEQ ID NO: 43 - protein, 515 aa, Golden hamster
MNPDLDTGHNTSAPAQWGELKDANFTGPNQTSSNSTLPQLDVTRAISVGLVLGAFILFAIVGNILVILSVACNRHLRTPTNYFIVNLAIADLLLSFTVLPFSATLEVLGYWVLGRIFCDIWAAVDVLCCTASILSLCAISIDHYIGVRYSLQYPTLVTRRKAILALLSVWVLSTVISIGPLLGWKEPAPNDDKECGVTEEPFYALFSSLGSFYIPLAVILVMYCRVYIVAKRTTKNLEAGVMKEMSNSKELTLRIHSKNFHEDTLSSTKAKGHNPRSSIAVKLFKFSREKKAAKTLGIVVGMFILCWLPFFIALPLGSLFSTLKPPDAVFKVVFWLGYFNSCLNPIIYPCSSKEFKRAFMRILGCQCRSGRRRRRRRRLGACAYTYRPWTRGGSLERSQSRKDSLDDSGSCMSGSQRTLPSASPSPGYLGRGAQPPLELCAYPEWKSGALLSLPEPPGRRGRLDSGPLFTFKLLGEPESPGTEGDASNGGCDATTDLANGQPGFKSNMPLAPGHF

SEQ ID NO: 44 - protein, 515 aa, Golden hamster
MNPDLDTGHNTSAPAQWGELKDANFTGPNQTSSNSTLPQLDVTRAISVGLVLGAFILFAIVGNILVILSVACNRHLRTPTNYFIVNLAIADLLLSFTVLPFSATLEVLGYWVLGRIFCDIWAAVDVLCCTASILSLCAISIDNYIGVRYSLQYPTLVTRRKAILALLSVWVLSTVISIGPLLGWKEPAPNDDKECGVTEEPFYALFSSLGSFYIPLAVILVMYCRVYIVAKRTTKNLEAGVMKEMSNSKELTLRIHSKNFHEDTLSSTKAKGHNPRSSIAVKLFKFSREKKAAKTLGIVVGMFILCWLPFFIALPLGSLFSTLKPPDAVFKVVFWLGYFNSCLNPIIYPCSSKEFKRAFMRILGCQCRSGRRRRRRRRLGACAYTYRPWTRGGSLERSQSRKDSLDDSGSCMSGSQRTLPSASPSPGYLGRGAQPPLELCAYPEWKSGALLSLPEPPGRRGRLDSGPLFTFKLLGEPESPGTEGDASNGGCDATTDLANGQPGFKSNMPLAPGHF

SEQ ID NO: 45 - protein, 359 aa, Rattus norvegicus
MALNSSAEDGIKRIQDDCPKAGRHSYIFVMIPTLYSIIFVVGIFGNSLVVIVIYFYMKLKTVASVFLLNLALADLCFLLTCPLWAVYTAMEYRWPFGNHLCKIASASVTFNLYASVFLLTCLSIDRYLAIVHPMKSRLRRTMLVAKVTCIIIWLMAGLASLPAVIHRNVYFIENTNITVCAFHYESRNSTLPIGLGLTKNILGFLFPFLIILTSYTLIWKALKKAYEIQKNKPRNDDIFRIIMAIVLFFFFSWVPHQIFTFLDVLIQLGVIHDCKISDIVDTAMPITICIAYFNNCLNPLFYGFLGKKFKKYFLQLLKYIPPKAKSHSSLSTKMSTLSYRPSDNMSSSAKKPASCFEVE

SEQ ID NO: 46 - protein, 346 aa, Homo sapiens
MMWGAGSPLAWLSAGSGNVNVSSVGPAEGPTGPAAPLPSPKAWDVVLCISGTLVSCENALVVAIIVGTPAFRAPMFLLVGSLAVADLLAGLGLVLHFAAVFCIGSAEMSLVLVGVLAMAFTASIGSLLAITVDRYLSLYNALTYYSETTVTRTYVMLALVWGGALGLGLLPVLAWNCLDGLTTCGVVYPLSKNHLVVLAIAFFMVFGIMLQLYAQICRIVCRHAQQIALQRHLLPASHYVATRKGIATLAVVLGAFAACWLPFTVYCLLGDAHSPPLYTYLTLLPATYNSMINPIIYAFRNQDVQKVLWAVCCCCAAARGRTPPSLGPQDESCTTASSSLAKDTSS

SEQ ID NO: 47 - DNA, 1041 bp, Homo sapiens
atgatgtggggtgcaggcagccctctggcctggctctcagctggctcaggcaacgtgaatgtaagcagcgtgggcccagcagaggggcccacaggtccagccgcaccactgccctcgcctaaggcctgggatgtggtgctctgcatctcaggcaccctggtgtcctgcgagaatgcgctagtggtggccatcatcgtgggcactcctgccttccgtgcccccatgttcctgctggtgggcagcctggccgtggcagacctgctggcaggcctgggcctggtcctgcactttgctgctgtcttctgcatcggctcagcggagatgagcctggtgctggttggcgtgctggcaatggcctttacygccagcatcggcagtctactggccatcactgtcgaccgctacctttctctgtacaatgccctcacctactattcagagacaacagtgacacggacctatgtgatgctggccttagtgtggggaggtgccctgggcctggggctgctgcctgtgctggcctggaactgcctggatggcctgaccacatgtggcgtggtttatccactctccaagaaccatctggtagttctggccattgccttcttcatggtgtttggcatcatgctgcagctctacgcccaaatctgccgcatcgtctgccgccatgcccagcagattgcccttcagcggcacctgctgcctgcctcccactatgtggccacccgcaagggcattgccacactggccgtggtgcttggagcctttgccgcctgctggttgcccttcactgtctactgcctgctgggtgatgcccactctccacctctctacacctatcttaccttgctccctgccacctacaactccatgatcaaccctatcatctacgccttccgcaaccaggatgtgcagaaagtgctgtgggctgtctgctgctgctgtgcggccgcacggggacgcaccccacccagcctgggtccccaagatgagtcctgcaccaccgccagctcctccctggccaaggacacttcatcgtga

SEQ ID NO: 48 - protein, 378 aa, Homo sapiens
MNASAASLNDSQVVVVAAEGAAAAATAAGGPDTGEWGPPAAAALGAGGGANGSLELSSQLSAGPPGLLLPAVNPWDVLLCVSGTVIAGENALVVALIASTPALRTPMFVLVGSLATADLLAGCGLILHFVFQYLVPSETVSLLTVGFLVASFAASVSSLLAITVDRYLSLYNALTYYSRRTLLGVHLLLAATWTVSLGLGLLPVLGWNCLAERAACSVVRPLARSHVALLSAAFFMVFGIMLHLYVRICQVVWRHAHQIALQQHCLAPPHLAATRKGVGTLAVVLGTFGASWLPFAIYCVVGSHEDPAVYTYATLLPATYNSMINPIIYAFRNQEIQRALWLLLCGCAAARGRTPPSLGPQDESCTTASSSLAKDTSS

SEQ ID NO: 49 - DNA, 1137 bp, Homo sapiens
atgaacgcgagcgccgcctcgctcaacgactcccaggtggtggtagtggcggccgaaggagcggcggcggcggccacagcagcaggggggccggacacgggcgaatggggaccccctgctgcggcggctctaggagccggcggcggagctaatgggtctctggagctgtcctcgcagctgtcggctgggccaccgggactcctgctgccagcggtgaatccgtgggacgtgctcctgtgcgtgtcggggacagtgatcgctggagaaaacgcgctggtggtggcgctcatcgcgtccactccggcgctgcgcacgcccatgttcgtgctggtaggcagcctggccaccgctgacctgttggcgggctgtggcctcatcttgcactttgtgttccagtacttggtgccctcggagactgtgagtctgctcacggtgggcttcctcgtggcctccttcgccgcctctgtcagcagcctgctggccattacggtggaccgctacctgtccctgtataacgcgctcacctattactcgcgccggaccctgttgggcgtgcacctcctgcttgccgccacttggaccgtgtccctaggcctggggctgctgcccgtgctgggctggaactgcctggcagagcgcgccgcctgcagcgtggtgcgcccgctggcgcgcagccacgtggctctgctctccgccgccttcttcatggtcttcggcatcatgctgcacctgtacgtgcgcatctgccaggtggtctggcgccacgcgcaccagatcgcgctgcagcagcactgcctggcgccaccccatctcgctgccaccagaaagggtgtgggtacactggctgtggtgctgggcactttcggcgccagctggctgcccttcgccatctattgcgtggtgggcagccatgaggacccggcggtctacacttacgccaccctgctgcccgccacctacaactccatgatcaatcccatcatctatgccttccgcaaccaggagatccagcgcgccctgtggctcctgctctgtggctgtgcggccgcacggggacgcaccccacccagcctgggtccccaagatgagtcctgcaccaccgccagctcctccctggccaaggacacttcatcgtga

SEQ ID NO: 50 - protein, 350 aa, Homo sapiens
MNEDLKVNLSGLPRDYLDAAAAENISAAVSSRVPAVEPEPELVVNPWDIVLCTSGTLISCENAIVVLIIFHNPSLRAPMFLLIGSLALADLLAGIGLITNFVFAYLLQSEATKLVTIGLIVASFSASVCSLLAITVDRYLSLYYALTYHSERTVTFTYVMLVMLWGTSICLGLLPVMGWNCLRDESTCSVVRPLTKNNAAILSVSFLFMFALMLQLYIQICKIVMRHAHQIALQHHFLATSHYVTTRKGVSTLAIILGTFAACWMPFTLYSLIADYTYPSIYTYATLLPATYNSIINPVIYAFRNQEIQKALCLICCGCAAARGRTPPSLGPQDESCTTASSSLAKDTSS

SEQ ID NO: 51 - DNA, 1053 bp, Homo sapiens
atgaatgaagacctgaaggtcaatttaagcgggctgcctcgggattatttagatgccgctgctgcggagaacatctcggctgctgtctcctcccgggttcctgccgtagagccagagcctgagctcgtagtcaacccctgggacattgtcttgtgtacctcgggaaccctcatctcctgtgaaaatgccattgtggtccttatcatcttccacaaccccagcctgcgagcacccatgttcctgctaataggcagcctggctcttgcagacctgctggccggcattggactcatcaccaattttgtttttgcctacctgcttcagtcagaagccaccaagctggtcacgatcggcctcattgtcgcctctttctctgcctctgtctgcagcttgctggctatcactgttgaccgctacctctcactgtactacgctctgacgtaccattcggagaggacggtcacgtttacctatgtcatgctcgtcatgctctgggggacctccatctgcctggggctgctgcccgtcatgggctggaactgcctccgagacgagtccacctgcagcgtggtcagaccgctcaccaagaacaacgcggccatcctctcggtgtccttcctcttcatgtttgcgctcatgcttcagctctacatccagatctgtaagattgtgatgaggcacgcccatcagatagccctgcagcaccacttcctggccacgtcgcactatgtgaccacccggaaaggggtctccaccctggctatcatcctggggacgtttgctgcttgctggatgcctttcaccctctattccttgatagcggattacacctacccctccatctatacctacgccaccctcctgcccgccacctacaattccatcatcaaccctgtcatatatgctttcagaaaccaagagatccagaaagcgctctgtctcatttgctgcggctgcgcggccgcacggggacgcaccccacccagcctgggtccccaagatgagtcctgcaccaccgccagctcctccctggccaaggacacttcatcgtga

SEQ ID NO: 52 - protein, 388 aa, Homo sapiens
MANTTGEPEEVSGALSPPSASAYVKLVLLGLIMCVSLAGNAILSLLVLKERALHKAPYYFLLDLCLADGIRSAVCFPFVLASVRHGSSWTFSALSCKIVAFMAVLFCFHAAFMLFCISVTRYMAIAHHRFYAKRMTLWTCAAVICMAWTLSVAMAFPPVFDVGTYKFIREEDQCIFEHRYFKANDTLGFMLMLAVLMAATHAVYGKLLLFEYRHRKMKPVQMVPAISQNWTFHGPGATGQAAANWIAGFGRGPMPPTLLGIRQNGHAASRRLLGMDEVKGEKQLGRMFYAITLLFLLLWSPYIVACYWRVFVKACAVPHRYLATAVWMSFAQAAVNPIVCFLLNKDLKKCLRTHAPCAAARGRTPPSLGPQDESCTTASSSLAKDTSS

SEQ ID NO: 53 - DNA, 1167 bp, Homo sapiens
atggccaacactaccggagagcctgaggaggtgagcggcgctctgtccccaccgtccgcatcagcttatgtgaagctggtactgctgggactgattatgtgcgtgagcctggcgggtaacgccatcttgtccctgctggtgctcaaggagcgtgccctgcacaaggctccttactacttcctgctggacctgtgcctggccgatggcatacgctctgccgtctgcttcccctttgtgctggcttctgtgcgccacggctcttcatggaccttcagtgcactcagctgcaagattgtggcctttatggccgtgctcttttgcttccatgcggccttcatgctgttctgcatcagcgtcacccgctacatggccatcgcccaccaccgcttctacgccaagcgcatgacactctggacatgcgcggctgtcatctgcatggcctggaccctgtctgtggccatggccttcccacctgtctttgacgtgggcacctacaagtttattcgggaggaggaccagtgcatctttgagcatcgctacttcaaggccaatgacacgctgggcttcatgcttatgttggctgtgctcatggcagctacccatgctgtctacggcaagctgctcctcttcgagtatcgtcaccgcaagatgaagccagtgcagatggtgccagccatcagccagaactggacattccatggtcccggggccaccggccaggctgctgccaactggatcgccggctttggccgtgggcccatgccaccaaccctgctgggtatccggcagaatgggcatgcagccagccggcggctactgggcatggacgaggtcaagggtgaaaagcagctgggccgcatgttctacgcgatcacactgctctttctgctcctctggtcaccctacatcgtggcctgctactggcgagtgtttgtgaaagcctgtgctgtgccccaccgctacctggccactgctgtttggatgagcttcgcccaggctgccgtcaacccaattgtctgcttcctgctcaacaaggacctcaagaagtgcctgaggactcacgccccctgcgcggccgcacggggacgcaccccacccagcctgggtccccaagatgagtcctgcaccaccgccagctcctccctggccaaggacacttcatcgtga

SEQ ID NO: 54 - protein, 388 aa, Homo sapiens
MANYSHAADNILQNLSPLTAFLKLTSLGFIIGVSVVGNLLISILLVKDKTLHRAPYYFLLDLCCSDILRSAICFPFVFNSVKNGSTWTYGTLTCKVIAFLGVLSCFHTAFMLFCISVTRYLAIAHHRFYTKRLTFWTCLAVICMVWTLSVAMAFPPVLDVGTYSFIREEDQCTFQHRSFRANDSLGFMLLLALILLATQLVYLKLIFFVHDRRKMKPVQFVAAVSQNWTFHGPGASGQAAANWLAGFGRGPTPPTLLGIRQNANTTGRRRLLVLDEFKMEKRISRMFYIMTFLFLTLWGPYLVACYWRVFARGPVVPGGFLTAAVWMSFAQAGINPFVCIFSNRELRRCFSTTLLYCAAARGRTPPSLGPQDESCTTASSSLAKDTSS

SEQ ID NO: 55 - DNA, 1167 bp, Homo sapiens
atggcgaactatagccatgcagctgacaacattttgcaaaatctctcgcctctaacagcctttctgaaactgacttccttgggtttcataataggagtcagcgtggtgggcaacctcctgatctccattttgctagtgaaagataagaccttgcatagagcaccttactacttcctgttggatctttgctgttcagatatcctcagatctgcaatttgtttcccatttgtgttcaactctgtcaaaaatggctctacctggacttatgggactctgacttgcaaagtgattgcctttctgggggttttgtcctgtttccacactgctttcatgctcttctgcatcagtgtcaccagatacttagctatcgcccatcaccgcttctatacaaagaggctgaccttttggacgtgtctggctgtgatctgtatggtgtggactctgtctgtggccatggcatttcccccggttttagacgtgggcacttactcattcattagggaggaagatcaatgcaccttccaacaccgctccttcagggctaatgattccttaggatttatgctgcttcttgctctcatcctcctagccacacagcttgtctacctcaagctgatatttttcgtccacgatcgaagaaaaatgaagccagtccagtttgtagcagcagtcagccagaactggacttttcatggtcctggagccagtggccaggcagctgccaattggctagcaggatttggaaggggtcccacaccacccaccttgctgggcatcaggcaaaatgcaaacaccacaggcagaagaaggctattggtcttagacgagttcaaaatggagaaaagaatcagcagaatgttctatataatgacttttctgtttctaaccttgtggggcccctacctggtggcctgttattggagagtttttgcaagagggcctgtagtaccagggggatttctaacagctgctgtctggatgagttttgcccaagcaggaatcaatccttttgtctgcattttctcaaacagggagctgaggcgctgtttcagcacaacccttctttactgcgcggccgcacggggacgcaccccacccagcctgggtccccaagatgagtcctgcaccaccgccagctcctccctggccaaggacacttcatcgtga

SEQ ID NO: 56 - protein, 364 aa, Homo sapiens
MQAAGHPEPLDSRGSFSLPTMGANVSQDNGTGHNATFSEPLPFLYVLLPAVYSGICAVGLTGNTAVILVILRAPKMKTVTNVFILNLAVADGLFTLVLPVNIAEHLLQYWPFGELLCKLVLAVDHYNIFSSIYFLAVMSVDRYLVVLATVRSRHMPWRTYRGAKVASLCVWLGVTVLVLPFFSFAGVYSNELQVPSCGLSFPWPERVWFKASRVYTLVLGFVLPVCTICVLYTDLLRRLRAVRLRSGAKALGKARRKVTVLVLVVLAVCLLCWTPFHLASVVALTTDLPQTPLVISMSYVITSLSYANSCLNPFLYAFLDDNFRKNFRSILRCAAARGRTPPSLGPQDESCTTASSSLAKDTSS

SEQ ID NO: 57 - DNA, 1095 bp, Homo sapiens
atgcaggccgctgggcacccagagccccttgacagcaggggctccttctccctccccacgatgggtgccaacgtctctcaggacaatggcactggccacaatgccaccttctccgagccactgccgttcctctatgtgctcctgcccgccgtgtactccgggatctgtgctgtggggctgactggcaacacggccgtcatccttgtaatcctaagggcgcccaagatgaagacggtgaccaacgtgttcatcctgaacctggccgtcgccgacgggctcttcacgctggtactgcccgtcaacatcgcggagcacctgctgcagtactggcccttcggggagctgctctgcaagctggtgctggccgtcgaccactacaacatcttctccagcatctacttcctagccgtgatgagcgtggaccgatacctggtggtgctggccaccgtgaggtcccgccacatgccctggcgcacctaccggggggcgaaggtcgccagcctgtgtgtctggctgggcgtcacggtcctggttctgcccttcttctctttcgctggcgtctacagcaacgagctgcaggtcccaagctgtgggctgagcttcccgtggcccgagcgggtctggttcaaggccagccgtgtctacactttggtcctgggcttcgtgctgcccgtgtgcaccatctgtgtgctctacacagacctcctgcgcaggctgcgggccgtgcggctccgctctggagccaaggctctaggcaaggccaggcggaaggtgaccgtcctggtcctcgtcgtgctggccgtgtgcctcctctgctggacgcccttccacctggcctctgtcgtggccctgaccacggacctgccccagaccccactggtcatcagtatgtcctacgtcatcaccagcctcagctacgccaactcgtgcctgaaccccttcctctacgcctttctagatgacaacttccggaagaacttccgcagcatattgcggtgcgcggccgcacggggacgcaccccacccagcctgggtccccaagatgagtcctgcaccaccgccagctcctccctggccaaggacacttcatcgtga

SEQ ID NO: 58 - protein, 419 aa, Homo sapiens
MCFSPILEINMQSESNITVRDDIDDINTNMYQPLSYPLSFQVSLTGFLMLEIVLGLGSNLTVLVLYCMKSNLINSVSNIITMNLHVLDVIICVGCIPLTIVILLLSLESNTALICCFHEACVSFASVSTAINVFAITLDRYDISVKPANRILTMGRAVMLMISIWIFSFFSFLIPFIEVNFFSLQSGNTWENKTLLCVSTNEYYTELGMYYHLLVQIPIFFFTVVVMLITYTKILQALNIRIGTRFSTGQKKKARKKKTISLTTQHEATDMSQSSGGRNVVFGVRTSVSVIIALRRAVKRHRERRERQKRVFRMSLLIISTFLLCWTPISVLNTTILCLGPSDLLVKLRLCFLVMAYGTTIFHPLLYAFTRQKFQKVLKSKMKKRVVCAAARGRTPPSLGPQDESCTTASSSLAKDTSS

SEQ ID NO: 59 - DNA, 1260 bp, Homo sapiens
atgtgtttttctcccattctggaaatcaacatgcagtctgaatctaacattacagtgcgagatgacattgatgacatcaacaccaatatgtaccaaccactatcatatccgttaagctttcaagtgtctctcaccggatttcttatgttagaaattgtgttgggacttggcagcaacctcactgtattggtactttactgcatgaaatccaacttaatcaactctgtcagtaacattattacaatgaatcttcatgtacttgatgtaataatttgtgtgggatgtattcctctaactatagttatccttctgctttcactggagagtaacactgctctcatttgctgtttccatgaggcttgtgtatcttttgcaagtgtctcaacagcaatcaacgtttttgctatcactttggacagatatgacatctctgtaaaacctgcaaaccgaattctgacaatgggcagagctgtaatgttaatgatatccatttggattttttcttttttctctttcctgattccttttattgaggtaaattttttcagtcttcaaagtggaaatacctgggaaaacaagacacttttatgtgtcagtacaaatgaatactacactgaactgggaatgtattatcacctgttagtacagatcccaatattctttttcactgttgtagtaatgttaatcacatacaccaaaatacttcaggctcttaatattcgaataggcacaagattttcaacagggcagaagaagaaagcaagaaagaaaaagacaatttctctaaccacacaacatgaggctacagacatgtcacaaagcagtggtgggagaaatgtagtctttggtgtaagaacttcagtttctgtaataattgccctccggcgagctgtgaaacgacaccgtgaacgacgagaaagacaaaagagagtcttcaggatgtctttattgattatttctacatttcttctctgctggacaccaatttctgttttaaataccaccattttatgtttaggcccaagtgaccttttagtaaaattaagattgtgttttttagtcatggcttatggaacaactatatttcaccctctattatatgcattcactagacaaaaatttcaaaaggtcttgaaaagtaaaatgaaaaagcgagttgtttgtgcggccgcacggggacgcaccccacccagcctgggtccccaagatgagtcctgcaccaccgccagctcctccctggccaaggacacttcatcgtga

SEQ ID NO: 60 - protein, 370 aa, Homo sapiens
MGQPGNGSAFLLAPNRSHAPDHDVTQQRDEVWVVGMGIVMSLIVLAIVFGNVLVITAIAKFERLQTVTNYFITSLACADLVMGLAVVPFGAAHILMKMWTFGNFWCEFWTSIDVLCVTASIETLCVIAVDRYFAITSPFKYQSLLTKNKARVIILMVWIVSGLTSFLPIQMHWYRATHQEAINCYANETCCDFFTNQAYAIASSIVSFYVPLVIMVFVYSRVFQEAKRQLQKIDKSEGRFHVQNLSQVEQDGRTGHGLRRSSKFCLKEHKALKTLGIIMGTFTLCWLPFFIVNIVHVIQDNLIRKEVYILLNWIGYVNSGFNPLIYCRSPDFRIAFQELLCARGRTPPSLGPQDESCTTASSSLAKDTSS

SEQ ID NO: 61 - protein, 382 aa, Homo sapiens
MDSSTGPGNTSDCSDPLAQASCSPAPGSWLNLSHVDGNQSDPCGLNRTGLGGNDSLCPQTGSPSMVTAITIMALYSIVCVVGLFGNFLVMYVIVRYTKMKTATNIYIFNLALADALATSTLPFQSVNYLMGTWPFGTILCKIVISIDYYNMFTSIFTLCTMSVDRYIAVCHPVKALDFRTPRNAKIVNVCNWILSSAIGLPVMFMATTKYRQGSIDCTLTFSHPTWYWENLLKICVFIFAFIMPILIITVCYGLMILRLKSVRMLSGSKEKDRNLRRITRMVLVVVAVFIVCWTPIHIYVIIKALITIPETTFQTVSWHFCIALGYTNSCLNPVLYAFLDENFKRCFREFCAAARGRTPPSLGPQDESCTTASSSLAKDTSS

SEQ ID NO: 62 - protein, 382 aa, Homo sapiens
MAPNTSTMDEAGLPAERDFSFRILTACFLSLLILSTLLGNTLVCAAVIRFRHLRSKVTNFFVISLAVSDLLVAVLVMPWKAVAEIAGFWPFGSFCNIWVAFDIMCSTASILNLCVISVDRYWAISSPFQYERKMTPKAAFILISVAWTLSVLISFIPVQLSWHKAKPTWPLDGNFTSLEDTEDDNCDTRLSRTYAISSSLISFYIPVAIMIVTYTSIYRIAQKQIRRISALERAAVHAKNCQTTAGNGNPVECAQSESSFKMSFKRETKVLKTLSVIMGVFVCCWLPFFISNCMVPFCGSEETQPFCIDSITFDVFVWFGWANSSLNPIIYAFNADFQKAFSTLLGCYRLCAAARGRTPPSLGPQDESCTTASSSLAKDTSS

SEQ ID NO: 63 - protein, 451 aa, Homo sapiens
MDVLSPGQGNNTTSPPAPFETGGNTTGISDVTVSYQVITSLLLGTLIFCAVLGNACVVAAIALERSLQNVANYLIGSLAVTDLMVSVLVLPMAALYQVLNKWTLGQVTCDLFIALDVLCCTSSILHLCAIALDRYWAITDPIDYVNKRTPRRAAALISLTWLIGFLISIPPMLGWRTPEDRSDPDACTISKDHGYTIYSTFGAFYIPLLLMLVLYGRIFRAARFRIRKTVKKVEKTGADTRHGASPAPQPKKSVNGESGSRNWRLGVESKAGGALCANGAVRQGDDGAALEVIEVHRVGNSKEHLPLPSEAGPTPCAPASFERKNERNAEAKRKMALARERKTVKTLGIIMGTFILCWLPFFIVALVLPFCESSCHMPTLLGAIINWLGYSNSLLNPVIYAYFNKDFQNAFKKIIKCNFCAAARGRTPPSLGPQDESCTTASSSLAKDTSS

SEQ ID NO: 64 - protein, 394 aa, Homo sapiens
MAPWPHENSSLAPWPDLPTLAPNTANTSGLPGVPWEAALAGALLALAVLATVGGNLLVIVAIAWTPRLQTMTNVFVTSLAAADLVMGLLVVPPAATLALTGHWPLGATGCELWTSVDVLCVTASIETLCALAVDRYLAVTNPLRYGALVTKRCARTAVVLVWVVSAAVSFAPIMSQWWRVGADAEAQRCHSNPRCCAFASNMPYVLLSSSVSFYLPLLVMLFVYARVFVVATRQLRLLRGELGRFPPEESPPAPSRSLAPAPVGTCAPPEGVPACGRRPARLLPLREHRALCTLGLIMGTFTLCWLPFFLANVLRALGGPSLVPGPAFLALNWLGYANSAFNPLIYCRSPDFRSAFRRLLCRCAAARGRTPPSLGPQDESCTTASSSLAKDTSS

SEQ ID NO: 65 - protein, 362 aa, Homo sapiens
MGPTSVPLVKAHRSSVSDYVNYDIIVRHYNYTGKLNISADKENSIKLTSVVFILICCFIILENIFVLLTIWKTKKFHRPMYYFIGNLALSDLLAGVAYTANLLLSGATTYKLTPAQWFLREGSMFVALSASVFSLLAIAIERYITMLKMKLHNGSNNFRLFLLISACWVISLILGGLPIMGWNCISALSSCSTVLPLYHKHYILFCTTVFTLLLLSIVILYCRIYSLVRTRSRRLTFRKNISKASRSSEKSLALLKTVIIVLSVFIACWAPLFILLLLDVGCKVKTCDILFRAEYFLVLAVLNSGTNPIIYTLTNKEMRRAFIRIMSCCKCAAARGRTPPSLGPQDESCTTASSSLAKDTSS